(19) United States
(12) Patent Application Publication
(10) Pub. No.: US 2006/0239926 A1
(43) Pub. Date: Oct. 26, 2006

(54) Specific high-relaxivity compounds

(75) Inventors: Marc Port, Deuil La Barre (FR); Olivier Rousseaux, Senlis (FR); Claire Corot, Lyon (FR); Philippe Prigent, Paris (FR); Eric Lancelot, Apremont (FR)

Correspondence Address: BIRCH STEWART KOLASCH & BIRCH, PO BOX 747, FALLS CHURCH, VA, 22040-0747, US

(73) Assignee: GUERBET, Villepinte (FR)

(21) Appl. No.: 10/560,830

(22) PCT Filed: Jun. 17, 2004
(86) PCT No.: PCT/IB04/02193

§ 371 Date: May 18, 2006

Related U.S. Application Data
(60) Provisional application No. 60/505,423, filed on Sep. 25, 2003.

(30) Foreign Application Priority Data
Jun. 25, 2003 (FR) 03/07694

(51) Int. Cl. A61K 49/10; A61K 31/555; C07F 5/00

(52) U.S. Cl. 424/9.363; 534/16; 514/185

(57) ABSTRACT

The invention relates to novel compounds that are useful for the diagnosis of many pathologies, in particular cardiovascular, cancer-related and inflammatory pathologies, and to pharmaceutical compositions comprising said compounds. These compounds comprise a component for targeting a pathological region, linked to a detection component which is effective in diagnostic terms.

The invention relates to novel compounds that are useful for the diagnosis of many pathologies, in particular cardiovascular, cancer-related and inflammatory pathologies, and to pharmaceutical compositions comprising said compounds. These compounds comprise a component for targeting a pathological region, linked to a detection component which is effective in diagnostic terms. The detection component is typically an MRI contrast agent, an X-ray contrast agent, or an entity containing a radioisotope or able to be detected by ultrasound or by optical imaging.

The administration of contrast products to patients contributes to improving the resolution of the images obtained and the accuracy of the diagnosis. Those skilled in the art are thus aware, for MRI (Magnetic Resonance Imaging), of a large-number of “non-specific” contrast products based on gadolinium chelates, which are linear or macrocyclic, described in particular in documents EP 71 564, EP 448 191, WO 02/48119, U.S. Pat. No. 6,399,043, WO 01/51095, EP 203 962, EP 292 689, EP 425 571, EP 230 893, EP 405 704, EP 290 047, U.S. Pat. No. 6,123,920, EP 292 689, EP 230 893, US2002/0090342, U.S. Pat. No. 6,403,055, WO 02/40060, U.S. Pat. No. 6,458,337, U.S. Pat. No. 6,264,914, U.S. Pat. No. 6,221,334, WO 95/31444, U.S. Pat. No. 5,573,752, U.S. Pat. No. 5,358,704 and US 2002/0127181, for example the compounds, DTPA, DTPA BMA, DTPA BOPTA, DO3A, TETA, TRITA, HETA, DOTA-NHS, TETA-NHS, DOTA (Gly)3-L-(p-isothiocyanoto)-Phe-amide, DOTA, M4DOTA, M4DO3A, M4DOTMA, MPDO3A, HBED, EHPG and BFCs (U.S. Pat. No. 6,517,814), compounds of the polypodal type. Such chelates are also, in certain cases, used as therapeutic products, in the form of radiopharmaceutical products.

However, it has become apparent that there is a need to develop “specific” imaging and treatment, the diagnostic contrast product or the therapeutic product being intended to target biological markers associated much more precisely with given pathologies. Several therapeutic fields are involved, in particular cardiovascular, cancer-related and inflammatory diseases. In the text, the term “specific product” is thus intended to mean a product which is capable of specifically targeting a biological marker associated with one or more pathologies, as opposed to a non-specific product, with no targeting of a biological marker, which may, in certain cases, give a signal in a pathological region, but which would also give this signal in a non-pathological region and therefore will not make it possible to accurately delimit the pathological region (for optimum removal, for example in the case of a tumour).

As regards the cardiovascular field and high-risk atheroma plaque, in terms of public health, vascular wall pathologies and the consequences thereof have an increasing incidence in the population. It appears to be particularly crucial for imaging techniques to allow, firstly, early diagnosis and screening of the regions at risk and, secondly, evaluation of the effectiveness of a treatment and therapeutic monitoring. Currently, more than a third of myocardial infarctions occur in asymptomatic patients and there are great expectations for the ability to predict the risk of cerebral or myocardial stroke in patients with atheroma. It is now accepted that investigating stenosis (anatomical imaging) is not predictive of this risk and that the stakes in terms of diagnosis and prognosis involve evaluating the functional state of the atheroma plaque. The availability of products enabling a method of evaluation which is predictive of atheroma plaque, for characterizing the wall, discriminating the constituents and evaluating the risks of rupture, thus enables targeted prevention in the patient at risk.

As regards the field of oncology, the cancer rate is high, with 10 million new cases diagnosed throughout the world in 1998, and it continues to increase due to the fact that the population is becoming older. At the worldwide level, 20 million new cases are anticipated in 2020 out of 8 billion individuals. Cancer is the third cause of mortality after cardiovascular and infectious diseases (and the second cause in developed countries). Today, the imaging tests available in the cancer field mainly allow the detection of suspect masses and do not provide any information regarding the cancerous or non-cancerous nature of these masses.

“Specific” imaging of pathological regions can be performed by MRI, X-rays, gamma-ray scintigraphy, CT scan, ultrasound, PET or optical imaging. In the case of MRI, a contrast is obtained by means of administering contrast agents containing paramagnetic or superparamagnetic metals which have an effect on the relaxivity of the protons from water. In the case of scintigraphy, the contrast is obtained by the specific localization of a radiopharmaceutical compound emitting gamma- or beta-rays.

The binding of contrast products or of radiopharmaceutical products to biological markers makes it possible to specifically target pathological regions. These biological molecules constitute biovectors of the contrast product or of the radiopharmaceutical product, capable of targeting markers associated with these pathological regions, hence the expression specific imaging. Suitable biovectors may, according to the type and the state of the pathology, be macromolecules such as antibodies or small molecules such as oligonucleotides, peptides, sugars or organic molecules, etc.

The association of biovectors with a contrast agent (MRI contrast agent, scintigraphy contrast agent, X-ray imaging contrast agent, ultrasound contrast agent, optical imaging contrast agent) or with a radioisotope that is therapeutically effective in radiotherapy (radioisotope emitting cytotoxic radiation) is thus known.

For MRI contrast products and radiopharmaceutical compounds, it is essential to obtain sufficient stability of the chelate-metal complex to avoid toxicity of the products.

The prior art thus mentions the association of abovementioned chelates with biovectors for targeting many pathologies, in particular cardiovascular, cancer-related, inflammatory or degenerative diseases.

For example, documents WO 99/59640 and WO 02/085908 mention the association of folate receptor-targeting derivatives with chelates of DOTA or DTPA type. Document WO 02/055111 describes the association of biovectors for targeting vitronectin, including αvβ3 and αvβ5, with chelates of DTPA type. Document WO 98/47541 describes the association of RGD peptide-type biovectors for targeting MMP, with chelates of DTPA type. The association of phosphonate or phosphinate biovectors with GdDTPA or a radionucleide (WO 02/062398), and compounds with a porphyrin backbone such as Gd2(DTPA)4-TPP, is also known.

The prior art describes very predominantly the association of many biovectors with chelates having a relatively low relaxivity, less than 10 mMol⁻¹Gd⁻¹s⁻¹, indicating that the imaging results obtained are satisfactory with this type of chelates.

This is, for example, the case of documents WO 01/97850, 6093,6157, U.S. Pat. No. 6,372,194, WO 2001/9188, WO 01/77145, WO 02 26776, WO 99/40947, WO 02062810, WO 02/40060, WO 92/09701, U.S. Pat. No. 6,537,520, U.S. Pat. No. 6,524,554, U.S. Pat. No. 6,489,333, U.S. Pat. No. 6,511,648, US A 2002/01068325, WO01/978611 WO 01/98294, WO 01/60416, WO 01/60280, WO01/97861, WO 02/081497, WO 01/10450, U.S. Pat. No. 6,261,535, U.S. Pat. No. 5,707,605, WO 02/28441, WO 02/056670, U.S. Pat. No. 6,410,695, U.S. Pat. No. 6,391,280, U.S. Pat. No. 6,491,893, US A 2002/0128553, WO 02/054088, WO 02/32292 and WO 02/38546.

Those skilled in the art were not led to search for modifications in the signal component (the chelate) since the biovector component was sufficiently effective for the diagnosis, the signal component being to some extent secondary.

It is recalled that the longitudinal relaxivity r₁ of a paramagnetic contrast product gives the measure of its magnetic efficiency and makes it possible to assess its influence on the signal recorded. In MRI medical imaging, the contrast products modify the proton relaxation time and the increase in relaxivity obtained makes it possible to obtain a higher signal. Gadolinium chelates, used in human clinics, such as Magnevist®, Dotarem® or Omniscan®, etc., have a low molecular mass and have molar relaxivities r₁ per G^{d} of less than 5 mM⁻¹s⁻¹.

In fact, several technical problems are not solved by this type of specific compound described in the prior art. These compounds are not satisfactory, or not sufficiently satisfactory, for obtaining the desired results under physiological conditions (in vivo) or under conditions similar to physiological conditions (ex vivo), because of a lack of signal and/or a lack of specificity and/or toxicity problems. Such chelates do hot make it possible to obtain sufficient relaxivity in imaging termed T1 imaging. Now, this T1 imaging is clearly the most common and the one most investigated by practitioners; it corresponds to a reading by means of a difference in positive contrast between a normal region and a pathological region: the visible signal is white in the pathological region, whereas the normal region appears grey.

More precisely:

1). The specificity of the diagnostic product does not make it possible to bring out differences between normal regions and pathological regions that are sufficiently significant to draw a conclusion regarding the precise delimitation of a pathological region, for example of a tumour. The affinity of the product for its target via the biovector is insufficient for an image that is relevant in diagnostic terms to be obtained.

2) The sensitivity of the product is insufficient: the signal provided by the product is insufficient for good imaging.

The inventors, as will be described in detail in the examples, have for example studied, as controls, products which associate folic acid and a chelate of DOTA type. Certain in vitro results on KB cells indicate targeting of cancer cells, as indicated in document WO 99/59640, but the MRI images obtained in vivo are unexploitable by the practitioner since the signal is insufficient. In order to be effective, such products would require very high doses with notable risks of toxicity, of receptor saturation, and of a pharmacological effect (and therefore of a side effect).

3) The product gives a signal that is a priori specific, but it is eliminated either too rapidly, which complicates the diagnosis, or too slowly, which leads to toxicity.

4) The specific contrast product does not make it possible to detect the pathological region or the region with the risk of becoming pathological (high-risk atheroma plaque, growing tumour, etc.) at a sufficiently early stage for upstream treatment. This is due in particular to the fact that the in vivo imaging signal is insufficient to detect small regions less than 5 mm.

5) The imaging parameters to be handled by the practitioner are complex during in vivo diagnosis. For example, the analysis: of the information may fluctuate greatly for small deviations in dose of contrast product administered, or depending on, the moment at which the signal is read relative to the administration of the product, which poses problems of organization and of reliability of the diagnosis and/or of the treatment.

6) The contrast product does not allow sufficiently targeted and selective detection of a pathological region. This is, for example, the case for the vulnerable high-risk plaques which are the cause of thromboses or atheroscleroses, as recalled in document US 2002/0127181. Many invasive or non-invasive techniques have thus been developed for monitoring the progression of the pathology including coronary angiography, intravascular angioscopy, intravascular MRI. For example, angiography can underestimate the degree of stenosis; invasive angioscopy or MRI with current contrast products makes it possible to visualize plaques, but not to distinguish between stable plaques and high-risk plaques.

Some documents, which are much fewer in number, such as U.S. Pat. No. 6,221,334, describe compounds which associate chelates having a relatively high relaxivity and biovectors. However, the compounds truly exemplified pose complex problems, in particular for manufacture. They are compounds of dendrimer type, described in Invest. Radiol, 35, 50-57, 2000, and the relaxivity of which is r1=9.3 mM⁻¹s⁻¹Gd⁻¹.

The chemical synthesis of such dendrimers with varied biovectors is also difficult.

All these problems require the structure of the contrast products to be further improved in order to make them completely effective, which is far from evident for those skilled in the art. The absence of specific MRI products in clinical trials, although the principle of associating a biovector and a paramagnetic chelate was put forward several years ago and has produced many studies, is, moreover, proof of this.

The invention is directed towards overcoming at least partly the disadvantages of the prior art. The inventors have succeeded in obtaining new compounds by optimizing, firstly, the signal component (contrastophore) using suitable high-relaxivity (HR) derivatives having a structure very different from the dendrimers, which makes it possible to limit the problems of complex manufacturing and of impurities, the problems of specificity and of toxicity due to high doses of biovectors, and the problems of insufficient signal and, secondly, the biovector component using biovectors whose affinity for the target ligands is sufficient to obtain a selective biodistribution capable of differentiating the pathological regions.

In particular, the products obtained have a very good molar relaxivity r1 in the magnetic fields commonly used, until now not obtained for specific products. The molar efficacy (r1 per Gd) is at least 25 to 40 mM⁻¹s⁻¹ for the monometallic derivatives described below (HR DOTA and HR PCTA in particular) and can reach values of 120 to 160 mM⁻¹s⁻¹ for the polymetallic derivatives described below, or even more of the order of 200 to 300 mM⁻¹s⁻¹, compared with values of the order of 5 to 9 mM⁻¹s⁻¹ with most of the derivatives of the prior art. It is recalled that obtaining a substantial signal by virtue of a high relaxivity makes it possible to obtain better spatial resolution.

In other words, the dose of biovector required to obtain the same signal is reduced accordingly, which makes it possible to greatly limit the dose of biovector used, and therefore the risks of toxicity and the side effects engendered by certain biovectors, and also the cost of manufacture, and to avoid using very complex biovectors. For the same amount of Gd injected into a patient, the compounds obtained by the inventors require a dose of biovector which is of the order of 10 to 100 times lower.

Compared to specific products with dendrimers (r1=9 mM⁻¹s⁻¹Gd⁻¹) onto which are grafted about thirty Gd atoms, the compounds obtained by the inventors (r1=25 to 40 mM⁻s⁻Gd⁻¹) make it possible to use, to obtain the same relaxivity (and therefore the same signal), a dose of Gd which is 3 to 4 times lower, which is a very great advantage in the imaging field.

These results are all the more advantageous since they are obtained using a chemical “platform” (the contrastophore) which can accept very varied biovectors.

Other technical advantages are mentioned later in the application, and are the result of the broad possible choice of biovectors and of the low toxicity of the products obtained.

For this, the applicant has used high-relaxivity, referred to as HR, chelates, which were partly described by the applicant in the granted patents EP B 661 279, EP B 922 700 and EP B 1 183 255, to obtain a satisfactory relaxivity (for a sufficient signal). These HR compounds are chelates, capable of forming paramagnetic gadolinium complexes in the case of MRI, containing a nitrogenous macrocycle bearing, on the nitrogen atoms, acetic groups characterized by the presence, on the carbon atom in the position alpha to the carboxyl, hydrophilic groups.

This association of at least one biovector with at least one HR chelate compound via at least one linker L made it necessary to overcome not only technical problems related to diagnosis and to biocompatibility mentioned above, but also technical problems related to the chemical structure of the products and described later for the various classes of the biovectors developed.

In addition, the inventors went against the technical bias according to which it is preferable to use small molecules for specific medical imaging products. In fact, they were able to note that the steric hindrance of the HR chelates used does not impair the affinity of the specific product for its target. Despite a molecule weight of the order of 8 to 20 KD, the product effectively reaches its specific targeting site.

Throughout the text, in the interests of simplicity, these compounds will, without distinction, be referred to as (BIOVECTOR)ₓ-L_{z}-(HR CHELATE)_{y} or; HR-BIOVECTOR. The term “HR CHELATE” can be replaced with the abbreviation HR Ch.

According to a first aspect, the invention therefore relates to compounds of general formula (E) below:

(1)
Bₓ-L_{z}-(HRCh)_{y} (E)

in which:
B is a biovector
L is a linker
HR Ch represents a chelate of formula (I):
   [(D)_{q}-(I_{a,b,c,d,e,f,g})ᵣ];

With:
a) I_{a,b,c,d,e,f,g} chosen from Iₐ, I_{b}, I_{c}, I_{d}, Iₑ, I_{f}, I_{g},
Iₐ, I_{b}, I_{c} having the meanings:

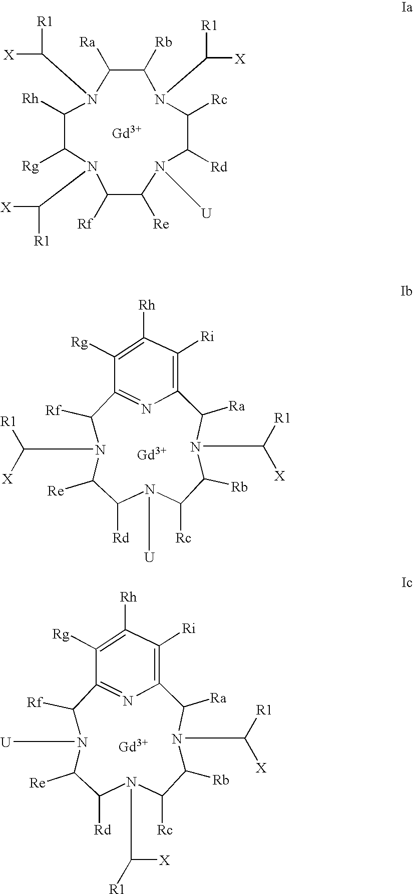

where:
the X, which may be identical or different, are chosen from CO₂R′ₐ, CONR′_{b}R′_{c} or P(R′_{d})O₂H, with:
   R′ₐ, R′_{b} and R′_{c}, which may be identical or different, representing H or (C₁-C₈) alkyl, which is optionally hydroxylated;
   P is the phosphorus atom, R′_{d} is chosen from OH, (C₁-C₈)alkyl or (C₁-C₈)alkoxy, (C₁-C₈)arylalkyl or (C₁-C₈)alkoxyalkyl;
R₁ represents a hydrophilic group typically of molecular weight (molar mass in g/mol) greater than 200), preferably of molecular weight greater than 300, more preferably greater than 500, even more preferably greater than 800, and better still greater than 1000), comprising at least three oxygen atoms, selected from groups:
   polyoxy(C₂-C₃)alkylene (i.e polyoxyethylenes and polyoxypropylenes), in particular polyethylene glycol and its C₁-C₃ monoethers and monoesters, preferably of molecular mass from 1000 to 2000
   polyhydroxyalkyl
   polyol (including functionalized oligosaccharides [this type of functionalization being described in particular in J. Polymer. Sc. Part A Polymer chemistry 23 1395-1405 (1985) and 29, 1271-1279 (1991) and in Bioconjugate chem. 3, 154-159 (1992)])
   R₂g)ₑ[(R₂g)ᵢR₃]ₕ where:
      h=1 or 2; i=0, 1 or 2; e=1 to 5
      R₂ represents (the R₂ being identical or different):
         nothing, an alkylene, an alkoxyalkylene, a polyalkoxyalkylene;
         a phenylene, or a heterocyclic residue which may be saturated or unsaturated, optionally substituted with OH, Cl, Br, I, (C₁-C₈)alkyl, (C₁-C₈)alkyloxy, NO2, NR_{X}R_{Y}, NR_{X}COR_{Y}, CONR_{X}R_{Y} or COOR_{X}, R_{X} and R_{Y} being H or (C₁-C₈)alkyl, and the linear, branched or cyclic C₁-C₁₄ alkyl, alkylene and alkoxy groups possibly being hydroxylated;
      g represents (the g being identical or different): nothing or a function O, CO, OCO, COO, SO₃, OSO2, CONR′, NR′CO, NR′COO, OCONR′, NR′, NR′CS, CSNR′, SO2NR′, NR′SO2, NR′CSO, OCSNR′, NR′CSNR′, P(O)(OH)NR′, NR′P(O)—(OH), in which R′ is H, (C₁-C₈)alkyl or R₃;
      R₃ represents alkyl, phenyl, alkyl substituted or interrupted with one or more phenyl groups, alkyleneoxy groups; amino or amido unsubstituted or substituted with alkyl optionally substituted or interrupted with one of the above groups; phenyl, phenylene and heterocyclic groups which may be substituted with OH, Cl, Br, I, (C₁-C₈)alkyl, (C₁-C₈)alkyloxy, NO2, NR_{X}R_{Y}, NR_{X}COR_{Y}, CONR_{X}R_{Y} or COOR_{X}, R_{X} and R_{Y} being H or (C₁-C₈)alkyl, and linear, branched or cyclic C₁-C₁₄ alkyl, alkylene and alkoxy groups which may be, hydroxylated;
   Rₐ to Rᵢ (i.e. Ra, Rb, Rc, Rd, Re, Rf, Rg, Rh, Ri) independently represent H, alkyl, hydroxyalkyl, alkylphenyl or cycloalkyl.
   U is a group —CXR₄-linker 1, CHR₄CON-linker 1, CHR₄—CHR₅OH-linker 1
   R₄ and R₅ independently representing H, alkyl or hydroxyalkyl,
   X having the meaning above,
   linker 1 being the linker providing the link between the HR Ch chelate and the linker L when q=0 and between the HR Ch chelate and D when q=1
I_{d}, Iₑ, I_{f} having the meanings: 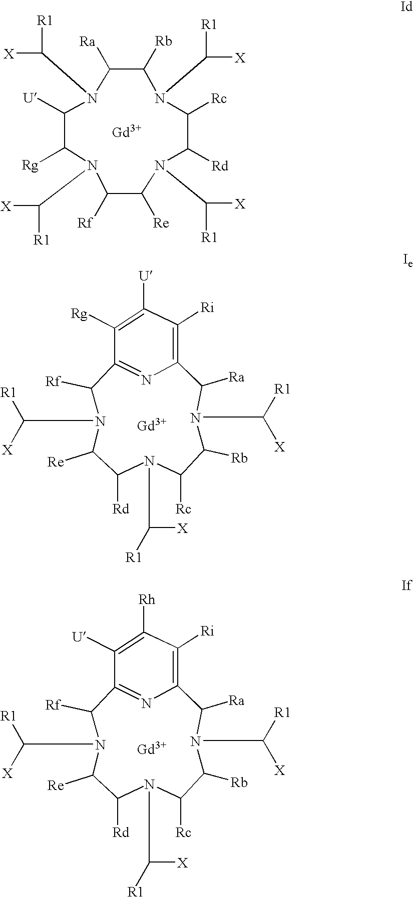
   —X, R1, Ra to Ri having the same meaning as above,
   U′ is linker 1,
I_{g} represents —

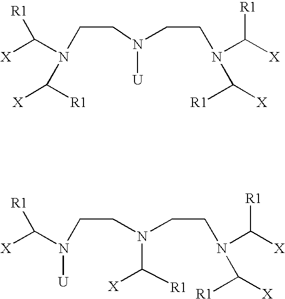

U, X, R1 having the same meaning as above.
b)
   q=0 or q=1
   r=1 when q=0, or r is between 2 and 5 when q=1
c) D is a polyfunctional molecule capable of linking the linker L to at least two HR chelates, D being capable of bonding to L via a linker 2 and to at least two metal chelates via linkers 1.
d) x, y and z are between 1 and 6, preferably x=1 to 3, y=1 to 8, z=1 to 3, given that y=z.

The linkers L are identical or different to one another, as are the HR Ch.

(E), in its form bound to an element M, is written B_{X}-L_{Z}-(HR Ch-M)_{y}; given that M is either a radionucleide, typically chosen from ⁹⁹Tc, ¹¹⁷Sn, ¹¹¹In, ⁹⁷R, ⁶⁷Ga, ⁶⁸Ga, ⁸⁹Zr, ¹⁷⁷Lu, ⁴⁷Sc, ¹⁰⁵Rh; ¹⁸⁸Re, ⁶⁰Cu, ⁶²Cu, ⁶⁴Cu, ⁶⁷Cu, ⁹⁰Y, ¹⁵⁹Gd, ¹⁴⁹Pr and ¹⁶⁶Ho, or a paramagnetic metal ion having the atomic number 21-29, 42-44, or 58-70, or a heavy metal ion having the atomic number 21-31, 39-49, 50, 56-80, 82, 83 or 90.

The invention also relates to the salts of the compounds of formula (E) with mineral or organic acids or bases, in particular the hydrochlorides of the amino groups and the sodium, potassium and N-methylglucamine salts of the carboxylic acid groups present on the chelates.

The groups —CR1X— constitute hydrophilic branches grafted onto the Gd core. Advantageously, these branches were chosen from:
the branches denoted AAG1′ AA28Br, AAG1 AA29Br described later;
the branches described in documents EP 661 279, EP 922 700, EP 1 183 255;
the “flash” branches described later,
the CO—NH-φ-CO—NH “rigid linker” branches, referred to as P792, mentioned later.

Such hydrophilic branches forming side arms on the acid groups may be different in nature and are intended to decrease the freedom of movement of the paramagnetic complex and of the paramagnetic ion which is attached thereto, the rotation of which in the magnetic field (inverse function of r1) is thus reduced, hence a phenomenon of complex immobilization.

In addition, quite surprisingly, these hydrophilic branches make it possible to conserve the initial affinity of the biovector. This appears to be due to an effect of formation of a network of water and of masking of charges, the specific interaction between the biovector and its target site not being impaired despite the size of the HR chelate, which size would have dissuaded those skilled in the art from coupling it to a biovector. In addition, this hydrophilicity makes it possible to resolve the considerable problems of lack of solubility encountered with the biovectors in the prior art. For the hydrophilic branches, a molecular mass of at least 200 has been indicated; it is clear to those skilled in the art that they may be led to vary this value slightly, provided that the effect of immobilizing the branches responsible for the high relaxivity is achieved. Typically, the molecular mass of the branches is less than 3000 in order to avoid having chains which are too complex to produce, and to have good weight efficiency with respect to Gd.

Moreover, the applicant makes the following comments:
For U═CXR4-CHR5OH-linker 1 or U═CHR4CON-linker 1, the synthesis is facilitated.
Preferably, X represents CO2R′ₐ, however, the use of CONR′bR′c makes it possible to obtain non-ionic compounds which are advantageous for decreasing the osmolality of the product, and the use of P(R′_{d})O₂H can make it possible to obtain products with higher relaxivity.
Preferably, Ra, Rb and Rc represent H, but it is also possible to use alkyl or cycloalkyl groups to stabilize the structure and to improve the relaxivity, on condition that they do not interfere with the desired properties of the product (rigidification by grafting alkyl groups is known to those skilled in the art in Inorganic Chemistry, vol 41, No. 25, p 6846-6855, 2002). Those skilled in the art are aware that hydroxyalkyl groups are known to decrease the toxicity of structures, as described in Inorganic Chemical Acta 317, 2001, 218-229, and Coordination Chemistry Reviews, 185-186, 1999, 451-470.

According to non-limiting embodiments, compounds (2) to (18) below will be obtained:

(2) the compounds (E) above in which R1 is (CH2)ₓCONHR with x=1, 2 or 3 and R is a hydrophilic group of molecular weight greater than 200, chosen from:

1) a group: 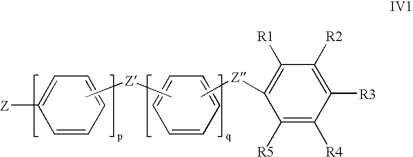
and Z is a bond, CH₂, CH₂CONH or (CH₂)₂NHCO
Z′ is a bond, O, S, NQ, CH₂, CO, CONQ, NQCO, NQ-CONQ or CONQCH₂CONQ,
Z″ is a bond, CONQ, NQCO or CONQCH₂CONQ
p and q are integers, the sum of which is 0 to 3;
R₁, R₂, R₃, R₄ or R₅ represent:
   either, independently of one another, H; Br, Cl, I, CONQ₁Q₂ or NQ₁COQ₂ with Q₁ and Q₂, which may be identical or different, being H or a (C₁-C₈)alkyl group which is mono- or polyhydroxylated or optionally interrupted with one or more oxygen atoms, and at least one and no more than two of R₁ to R₅ are CONQ₁Q₂ or NQ₁COQ₂;
   or R₂ and R₄ represent 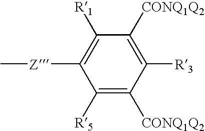
   and R₁, R′₁, R₃, R′₃, R₆ and R′₅, which may be identical or different, represent H, Br, Cl or I, Q₁ and Q₂ have the same meaning as above and Z′″ is a group chosen from CONQ, CONQCH₂CONQ, CONQCH₂, NQCONQ and CONQ(CH₂)₂NQCO and Q is H or (C₁-C₄)alkyl, which is optionally hydroxylated, it being possible for the alkyl groups to be linear or branched;

2) a “flash” branch 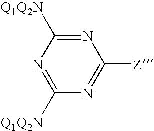
with Z″″ being NQ(CH₂)ⱼ(CH₂OCH₂)ᵢ(CH₂)ⱼNH₂ with i=2 to 6 and j=1 to 6,
preferably 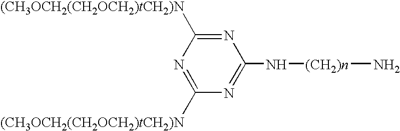
or 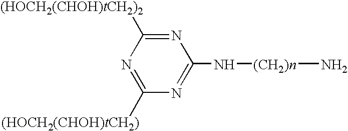
with t=1, 2, 3 or 4 and n=2 to 6.

(3) the compounds (E) with q=1.

One or more HR chelates of the compound (E) thus provide a divider D. Various dividers are possible, as long as they make it possible to provide the link between, firstly, at least two chelates and, secondly, the linker(s) L_{Z}. Various polyfunctional backbones can be used, by those skilled in the art, as a divider, described in particular in Chemical Reviews, 2001, 101 (12), 3819-386 and Topics in Current Chemistry, vol. 217, 212, 210, 197. Preferred dividers are aromatic backbones polyfunctionalized with carboxylate and/or amino groups.

D may be written in the form (Div-linker 2), Div being a group having a number of free valences at least equal to r. D is bonded, firstly, to at least two metal chelates via linkers 1 mentioned above and, secondly, to linker L via a linker 2. This gives, for example, for r=2
(linker 2)-Div-(linker 1)₂;
(E) being written:
   (BIOVECTOR)ₓ-L_{z}-[(Linker2-Div)_{q}-(I_{a,b,c,d,e,f,g})ᵣ]_{y}
the two linkers I being included in I_{a,b,c,d,e,f,g,h}.

Unlike the case of the dendrimers, the compounds (E), in particular those with a divider D, make it possible to obtain:
stearic separation of the signal component and the biovector component,
conformational freedom of the biovector, which, since it is not prestressed, conserves its affinity,
an effective signal with a limited number of Gd,
control of the purity and of the polydispersity, whereas the purity of the dendrimer-type products is very low, less than 5%,
a completely controlled number of Gd administered,
a hydrophilicity of the contrastophore component which makes the component inert, thus promoting recognition of the biovector on its specific binding site.

In addition
the scheme for constructing the products obtained with or without divider is modular, it being possible to control the physicochemical properties of each module (B, L, HR Ch) of the structure, for example the hydrophilicity, the viscosity, the charge, which makes it possible to control in particular the solubility of the product, and to limit the amount of product to be administered;
unlike the dendrimers, unwanted effects of binding of molecules (cholesterol, ions, endogenous metals, etc.) are avoided.

Furthermore, the products obtained appear to have the advantage of exhibiting in the organism a remanence greater than that of specific compounds of non-HR DOTA-type of the prior art: the size of the products obtained decreases their elimination, in particular in the kidneys, contributing to increasing the contact time in the organism.

This structure with divider (the compounds are then called polymetallic since they carry several HR chelates) is particularly advantageous since it makes it possible to further obtain a large increase in the molar relaxivity, and therefore in the effectiveness of the product for the same dose of Gd administered to the patient. The relaxivity per chelate, of the order of 25 mM⁻¹s⁻¹ for HR DOTAs to 40 mM⁻¹s⁻¹ for HR PCTAs, is multiplied by the number of chelates in the structure. In other words, for an HR-biovector bearing four chelates, for example, the relaxivity is of the order of 120 to 160. This structure can make it possible to obtain very good results even with biovectors for which the non-HR derivatives are not effective enough in imaging. The polymetallic biovectors typically bear from 2 to 8 gadolinium chelates, or even more. Compounds with the following architecture will be obtained according to preferred embodiments:
x=y=z=1 and q=1: biovector linked via a linker L to a divider, itself bonded to two chelates,
x=1, y=z=2, and q=1: biovector connected via two linkers L to two dividers, each divider being bonded to two chelates (therefore 4 chelates in total)
x=1, y=z=3, and q=1: biovector connected via three linkers L to three dividers, each divider being bonded to two chelates (therefore 6 chelates in total).

It is also understood that a divider D can itself in fact comprise at least two subdividers in arborescent form. There will be, for example, the case of a biovector linked via two linkers L to two dividers, each divider comprising two subdividers and thus bearing 4 chelates, the polymetallic biovector containing a total of 8 chelates.

(4) the compounds of formula (E) with HR Ch representing a group chosen from: 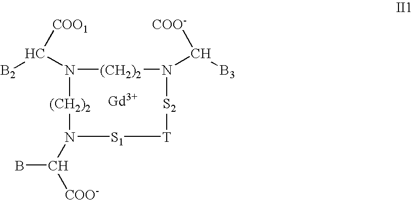
in which:
—S₁-T-S₂— is
   1) either 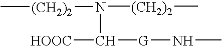
      where S₁═S₂═(CH₂)₂
   with all three of B₁, B₂ and B₃ representing (CH₂)ₓCONHR with x=1, 2 or 3
   2) or 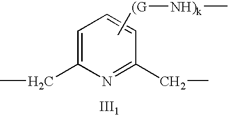
   with k=0 and S₁═S₂═CH₂
   one of B1, B2, B3 representing G-NH, and the others representing (CH₂)ₓCONHR
   3) or 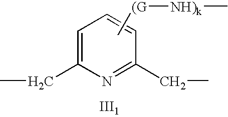
      with k=1
   all three of B₁, B₂, B₃ representing (CH₂)ₓCONHR with x=1, 2 or 3
      and GNH chosen from:
the groups —(CH₂)ₙ—NH— with n=1 to 4,
   or 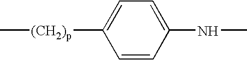
      with p=0 to 3;
      G-NH represents linker 1 described above in these compounds.

(5) the compounds of formula (E) with HR Ch representing a group chosen from:

1) the group 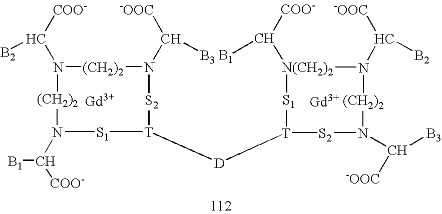
in which
—S₁-T-S₂— is 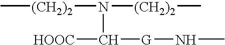
where S₁═S₂═(CH₂)₂
all three of B₁, B₂, B₃ representing (CH₂)ₓCONHR with x=1, 2 or 3

2) the group 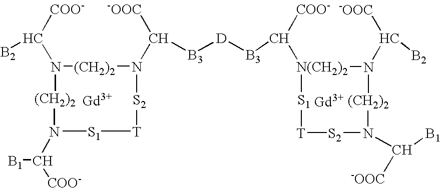
IIa2 (compound referred to as N-functionalized PCTA)
or IIb2 (compound referred to as N-functionalized PCTA and positional isomer of IIb2) 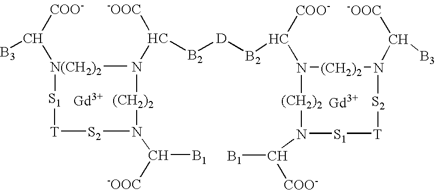
IIb2
in which S₁-T-S₂— is: 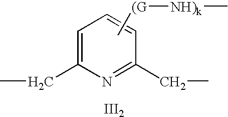
with k=0 and S₁=S₂═CH₂;
B₃ representing G-NH, and B1 and B2 representing (CH₂)ₓCONHR for IIa2
B₂ representing G-NH, and B1 and B3 representing (CH₂)ₓCONHR for IIb2

3) the group 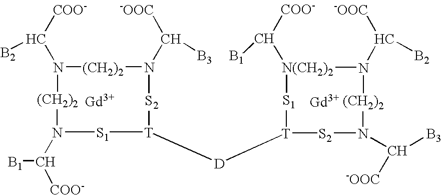
IIc2 (compound referred to as C-functionalized PCTA)
when S₁-T-S₂— is: 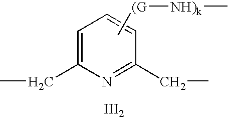
   with k=1 and S₁═S₂═CH₂;
all three of B₁, B₂, B₃ representing (CH₂)ₓCONHR with x=1, 2 or 3 for IIc2
   given that, for II2, IIa2, IIb2 and IIc2,
GNH is chosen from the groups —(CH₂)ₙ—NH— with n=1 to 4,
where 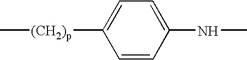
   with p=0 to 3;
(6) The compounds of formula (E) with D being an aromatic backbone polyfunctionalized with carboxylate and/or amino groups, preferably Div being of the 1,3,5-triazine type, of formula: 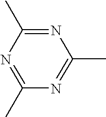
   with (linker 1)₂-Div-(linker 2) being written: 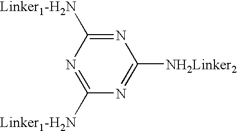
      with linker 1 and linker 2 being chosen from a) and b), and preferably a):
a) (CH₂)₂-φ-NH , (CH₂)₃—NH, NH—(CH₂)₂—NH, NH—(CH₂)₃—NH, nothing or a single bond,
b) P1-I-P2, which may be identical or different, P1 and P2 being chosen from O, S, NH, nothing, CO₂, NCS, NCO, SO₃H, NHCO, CONH, NHCONH, NHCSNH, SO₂NH—, NHSO₂—, squarate
   with I=alkylene, alkoxyalkylene, polyalkoxyalkylene, alkylene interrupted with phenylene, alkylidene, alkilidene, alkynylene,

D is, for example, according to one embodiment:

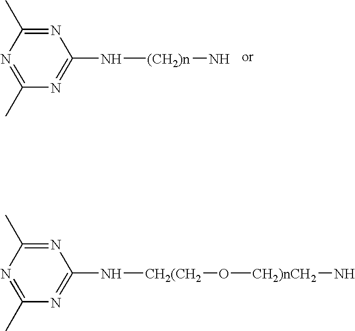

(7) The compounds of formula (E) with L being a linker chosen from polyoxyalkylenes, squaric acid, PEG-squarate assemblies, a radical: alkylene, alkoxyalkylene, polyalkoxyalkylene, alkylene interrupted with phenylene, alkylidene, alkilidene.

A large number of linkers L can be used, in so far as they are capable of interacting with at least one biovector functional group and at least one HR chelate functional group. Mention will in particular be made of:
a.1 (CH₂)₂-φ-NH , (CH₂)₃—NH, NH—(CH₂)₂—NH, NH—(CH₂)₃—NH, nothing or a single bond,
a.2 P1-I-P2, which may be identical or different, P1 and P2 being chosen from O, S, NH, nothing, CO₂, NCS, NCO, SO₃H, NHCO, CONH, NHCONH, NHCSNH, SO₂NH—, NHSO₂—, squarate,
   with I=alkylene, alkoxyalkylene, polyalkoxyalkylene, alkylene interrupted with phenylene, alkylidene, alkilidene;
b) linkers described in U.S. Pat. No. 6,264,914, capable of reacting with amino, hydroxyl, sulphhydryl, carboxyl, carbonyl, carbohydrate, thioether, 2-aminoalcohol, 2-aminothiol, guanidinyl, imidazolyl or phenol functional groups (of the biovector and of the chelate).
Groups capable of reacting with sulphhydryl groups include alpha-haloacetyl compounds of the type X—CH₂CO— (where X═Br, Cl or I), which can also be used to act with imidazolyl, thioether, phenol or amino groups.
Groups cap able of reacting in particular with amino groups include:
   alkylating compounds: alpha-haloacetyl compounds, N-maleiimide derivatives, aryl compounds (nitrohaloaromatic compounds, for example), aldehydes and ketones capable of forming Schiff bases, epoxide derivatives such as epichlorohydrin, derivatives of triazines containing chlorine which are very reactive with respect to nucleophiles, aziridines, squaric acid esters, alpha-haloalkyl ethers;
   acylating compounds: isocyanates and isothiocyanates, sulphonyl chlorides, esters such as nitrophenyl esters or N-hydroxysuccinimidyl esters, acid anhydrides, acyl azides, azlactones, imidoesters.
Groups capable of reacting with carboxyl groups include diazo compounds (diazoacetate esters, diazoacetamides), carboxylic acid-modifying compounds (carbodiimides, for example), isoxazolium derivatives (nitrophenyl chloroformate; carbonyldiimidazoles, etc.), quinoline derivatives.
Groups capable of reacting with guanidinyl groups include dione compounds such as phenylenediglyoxal, diazonium salts;
c) certain linkers described in U.S. Pat. No. 6,537,520 of formula (Cr₆r₇)_{g}—(W)ₕ—(Cr₆ₐr₇ₐ)_{g′}-(Z)ₖ—(W)_{h′}—(Cr₈r₉)_{g″}-(W)_{h′}-(Cr₈ₐr₉ₐ)_{g′″} with: −g+h+g′+k+h′+g″+h″+g′″ other than 0;
   W chosen from O, S, NH, NHC(═O), C(═O)NH, —C(═O), C(═O)O, OC(═O), —NHC(═S)NH, NHC(═O)NH, SO₂, (OCH₂CH₂)ₛ, (CH₂CH₂O)ₛ′, (OCH₂CH₂CH₂)_{s″}, (CH₂CH₂CH₂O)ₜ;
   Z chosen from the group: aryl substituted with 0-3 r₁₀, C₃-C₁₀ cycloalkyl substituted with 0-3 r₁₀, system of a heterocycle of 5-10 members containing 1-4 hetero atoms independently chosen from N, S, O and substituted with 0-3 r₁₀;
   r6, r6a, r7, r7a, r8, r8a, r9 and r9a independently chosen from: H, ═O, COOH, SO₃H, PO₃H, C₁-C₅ alkyl substituted with 0-3 r₁₀, aryl substituted with 0-3 r₁₀, benzyl substituted with 0-3 r₁₀, C₁-C₅ alkoxy substituted with 0-3 r₁₀, NHC(═O)r₁₁, C(═O)NH, r₁₁, NHC(═O)NH, r₁₁, NH r₁₁, r₁₁, and a linker with HR Ch;
   r₁₀ independently chosen from: a linker to ChRR, COOr₁₁, OH, NH r₁₁, SO₃H, PO₃H, aryl substituted with 0-3 r₁₁, C₁-C₅ alkyl substituted with 0-1 r₁₂, C₁-C₅ alkoxy substituted with 0-1 r₁₂, and a heterocycle of 5-10 members containing 1-4 hetero atoms independently chosen from N, S, O and substituted with 0-3 r₁₁;
   r₁₁ is independently chosen from: H, aryl substituted with 0-1 r₁₂, a heterocycle containing 5-10 members comprising 1-4 hetero atoms chosen from N, S, O, and substituted with 0-1 r₁₂, C₃-C₁₀ cycloalkyl substituted with 0-1 r₁₂, polyalkylene glycol substituted with 0-1 r₁₂, carbohydrate substituted with 0-1 r₁₂.
   r₁₂ is a linker with HR Ch;
   with k chosen from 0, 1, 2; h chosen from 0, 1, 2; h′ chosen from 0, 1, 2, 3, 4, 5; h″ chosen from 0, 1, 2, 3, 4, 5; g chosen from 0, 1, 2, 3, 4, 5, 6, 7, 8, 9, 10; g′ chosen from 0, 1, 2, 3, 4, 5, 6, 7, 8, 9, 10; g″ chosen from 0, 1, 2, 3, 4, 5, 6, 7, 8, 9, 10; g′″ chosen from 0, 1, 2, 3, 4, 5, 6, 7, 8, 9, 10; s chosen from 0, 1, 2, 3, 4, 5, 6, 7, 8, 9, 10; s′ chosen from 0, 1, 2, 3, 4, 5, 6, 7, 8, 9, 10; s″ chosen from 0, 1, 2, 3, 4, 5, 6, 7, 8, 9, 10; t chosen from 0, 1, 2, 3, 4, 5, 6, 7, 8, 9, 10;

d) certains linkers described in document WO 02/085908, for Example a linear or branched linker chain chosen from:
CR6′″R7′″-, —(R6′″)C═C(R7′″)═, —CC—, —C(O)—, —O—, —S—, —SO₂—, —N(R3′″)—, —(R6′″)C═N—, —C(S)—, —P(O0(OR3′″)—, —P(O)—(OR3′″)O—, with R′″3 a group capable of reacting with a nitrogen or an oxygen,
a cyclic region (divalent cycloalkyls, divalent heterocyclyls), —
polyalkylenes, polyalkylene glycols

e) linkers described in document WO 02/094873.

For the linker 1 and linker 2 linkers, a chemical bond or linkers from a) will typically be used.

(8) The compounds according to (3) to (7) with x of (CH₂)xCONHR being x=2.

(9) The compounds (4) to (8) in which —S₁-T-S₂— represents: 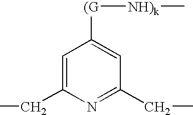
with S₁═S₂═CH₂.

(10) The compounds according to (9) of formula II.1 in which k is 1 and G is —(CH₂)₃—.

(11) The compounds according to (9) of formula II.1 in which k is 0 and B₂ or B₃ represents —(—CH₂)₃NH— or

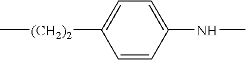

(12) The compounds according to (4) to (9), in which —S₁-T-S₂— represents: 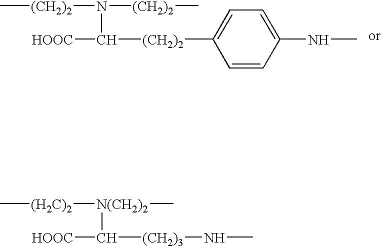
with S₁=S₂═(CH₂)₂.

(13) The compounds according to (4) to (9) for which B₁, B₂ and B₃, when they do not represent -G-NH, represent —(CH₂)₂CONHR, with, in R, p=q=0 and Z being —CH₂CONH.

(14) The compounds according to (13) for which R represents: 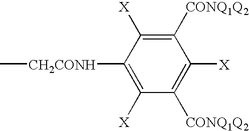
and the X are identical and represent Br or I, while Q₁ and Q₂, which may be identical or different, are mono- or polyhydroxylated (C₁-C₈)alkyl groups such that each CONQ₁Q₂ contains from 4 to 10 hydroxyls in total.

(15) The compounds according to (13) for which R represents: 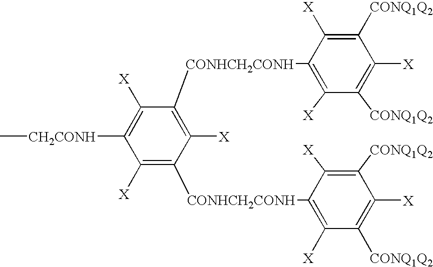
and the X, which are identical, are Br or I, and Q₁ and Q₂, which may be identical or different, are mono- or polyhydroxylated (C₁-C₈)alkyl groups such that each CONQ₁Q₂ group contains from 4 to 10 hydroxyls in total.

(16) The compounds according to (1) to (12) for which R represents:

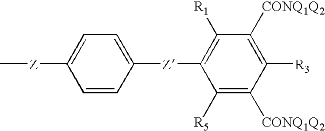

Z is CH₂ or CH₂CONH, Z′ is CONH or CONHCH₂CONH, and R₁, R₃, R₅, which are identical, are Br or I, and Q₁ and Q₂, which may be, identical or different, being mono- or polyhydroxylated (C₁-C₈)alkyl groups such that each CONQ₁Q₂ group contains from 4 to 10 hydroxyls in total.

(17) The compounds according to (1) to (12) for which R represents:

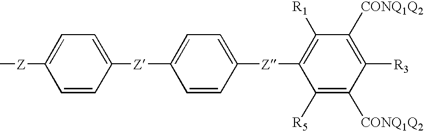

Z is CH₂CONH, Z′ is CONH, Z″ is CONHCH₂CONH and R₁, R₃, R₅, which are identical, are Br or I, and Q₁ and Q₂, which may be identical or different, are monohydroxylated or polyhydroxylated (C₁-C₈)alkyl groups such that each CONQ₁Q₂ group contains from 4 to 10 hydroxyls in total.

(18) The compounds according to (1) to (12) for which R represents: 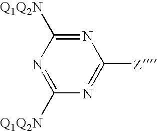
with Z″″ being NQ(CH₂)ⱼ(CH₂OCH₂)ᵢ(CH₂)ⱼNH₂, with i=2 to 6 and j=1 to 6,
preferably 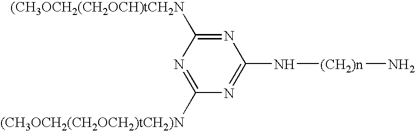
or 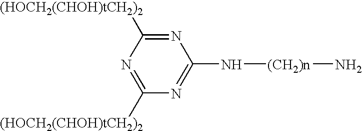
with t=1, 2, 3 or 4 and n=2 to 6.

The following compounds A1 in which x and R have the meanings above have in particular been synthesized:
those in which the macrocycle is cyclen, in which, in formula II1, —S₁-T-S₂—
represents: 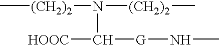
   which have the formula: 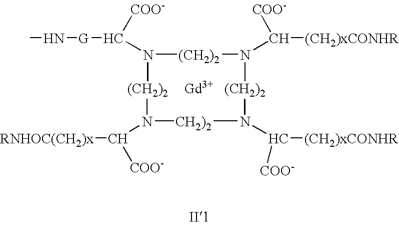
   with -G-NH being —(CH₂)₃—NH— or 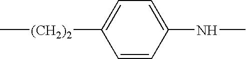
those in which the macrocycle is 3,6,9,15-tetraazabicyclo[9.3.1]pentadeca-1(15),11,13-triene, functionalized on one of the aliphatic nitrogen atoms of formula II1′ in which
   —S₁-T-S₂— represents: 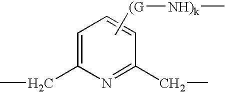
with k=0
of formula: 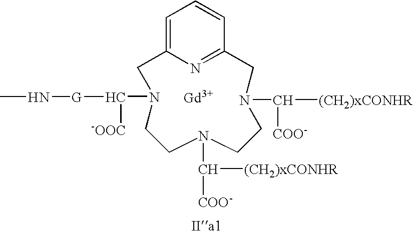
or: 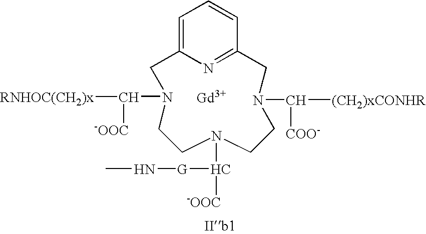
   with -G-NH=—(CH₂)₃—NH— or 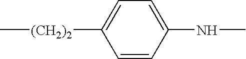
and those functionalized on the pyridyl ring of formula II1 in which —S₁-T-S₂— represents: 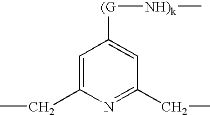
   with k=1 and G=(CH₂)₃;
of formula: 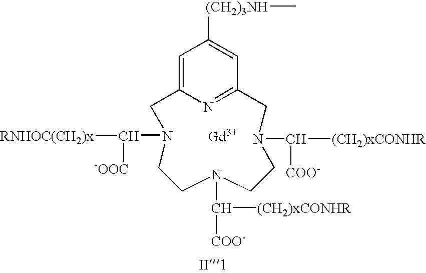
and especially the A1 residues in which x=2.

Similarly, compounds A2 similar to the above compounds II′1, II″a1, II″b1 and II″1, have been prepared by replacing the Gd monomer with a Gd dimer, respectively II′2, II″a2, II′b2, II′″2.

1) 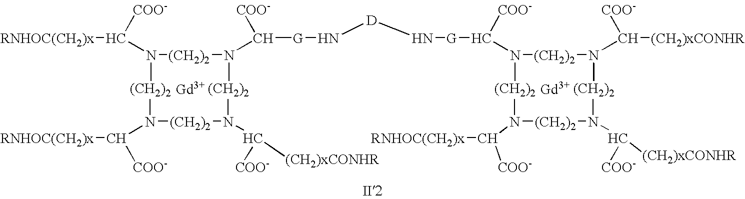
with -G-NH being —(CH₂)₃—NH— or 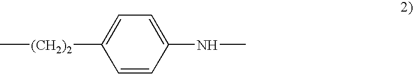 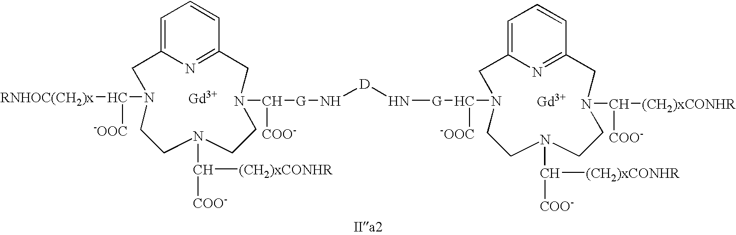
or II″b2 (positional isomer of II″a2) 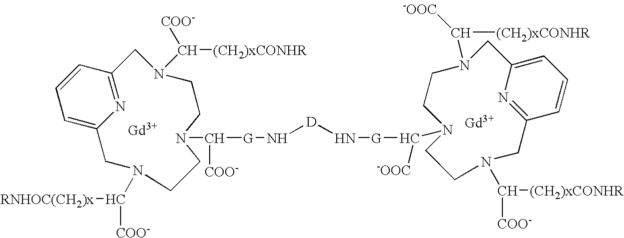
   with -G-NH being —(CH₂)₃—NH— or 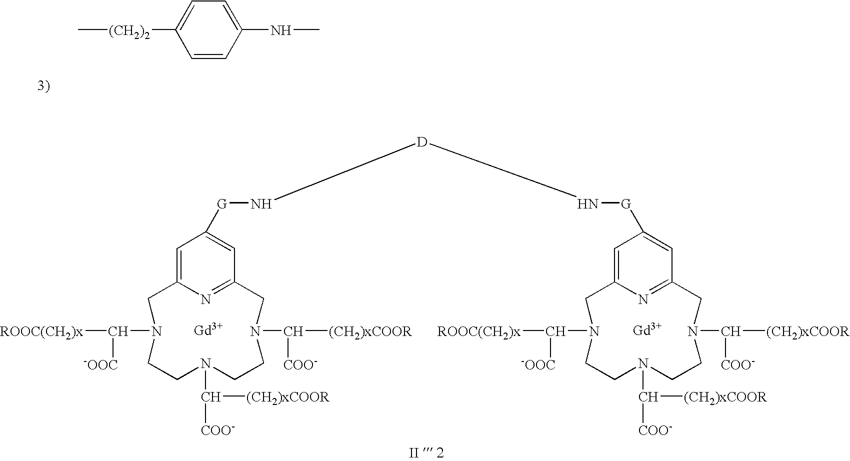
with G-NH being —(—CH₂)₃—NH,
and especially the A2 residues in which x=2.

The compounds of formulae II′₂, II″ₐ₂, II″_{b2} are obtained starting with two equivalents of the compounds of structure V₁ as defined in the application, by double substitution reaction on 2,4,6-trichloro-1,3,5-triazine in aqueous medium or in a mixture made up of water and a water-miscible polar solvent, controlling the pH and the temperature.

The residues of formula R— are introduced by peptide coupling, according to methods known to those skilled in the art, of the corresponding amines of formula R—NH₂, the structure which was defined above, for example in aqueous medium in the presence of a compatible coupling agent such as EDCI and, optionally, a catalyst.

The third chlorine atom is finally shifted by means of a large excess of diamine, for example of formula H₂N—(CH₂)ₐ—NH₂ or H₂N—CH₂—(CH₂—O—CH₂)_{b}CH₂—NH₂ with a=2 to 5 and b=1 to 4.

The compounds of formula II′″₂ are prepared starting with the aminated precursors derived from the residues of formula II′″₁ and of structure: 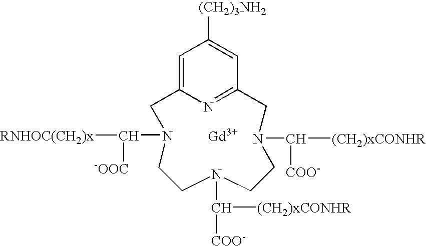
according to a similar protocol by double substitution of the triazine ring and shifting of the residual chlorine atom by means of a large excess of diamine as defined above.

To synthetize compounds A1, use has been made of precursors A′1 NH of formula: 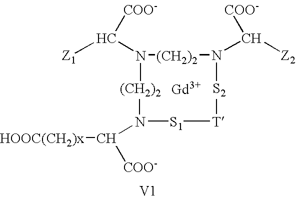
in which x=1, 2 or 3 and —S₁-T′-S₂— is: 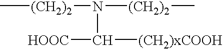
with S₁=S₂=(CH₂)₂
or 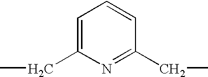
   with S═S₂═H₂
   and one of the groups Z₁ or Z₂ is chosen from the groups —(—CH₂)₃NH₂ or 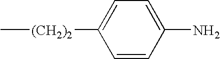
   in which the NH₂ group may be optionally protected in a conventional manner, in carbamate, phthalimide or benzylamine form as generally described in Protective Groups in Organic Synthesis, 3rd Ed. Ed. T. W. Greene, Pig. M. Wuts (J. Wiley) p. 494-653,
   and the other of Z₁ or Z₂ is (CH₂)ₓCOOH.

The compounds V1 of 1) are referred to as of HR DOTA-type, the compounds of 2) are referred to as of N-functionalized HR PCTA-type.

Use has also been made of precursors A′1NH of formula: 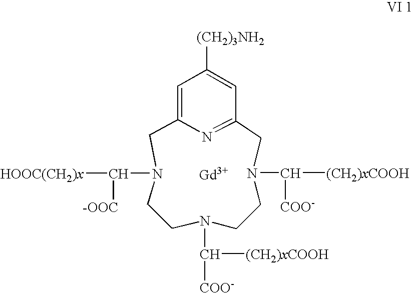
with x=1, 2, 3
in which the NH₂ group is optionally protected or salified,
and particularly the compounds V1 and VI1 in which x=2.

These compounds VI1 are referred to as HR PCTA of the C-functionalized type, the amine function being located on the external ring.

When the 1,3,5-triazino residue has been used as divider DIV, the precursor V1, VI1 or VI′1 is preferably reacted on 2,4,6-trichloro-1,3,5-triazine under usual conditions for a nucleophilic substitution in the presence of a base in an aprotic polar solvent, optionally as a mixture with water, in particular as described in Comprehensive Organic Chemistry, D. Bostow, W. Ollis, vol. 4, p. 150-152 (Pergamon Press) or in Tetrahedron Letters, 41(11), 2000, 1837-1840. The reaction may be carried out in the presence of an inorganic base such as NaOH or Na₂CO₃ or of a tertiary amine such as triethylamine, for example in water in the presence of 5 to 60% by volume of 1,6-dioxane, of tetrahydrofuran or of dimethylformamide.

To prepare the intermediate compounds of formula V1 in which Z₁ or Z₂ represents (CH₂)₃NH₂, it is possible to react, in a first stage, on the corresponding macrocyclic compound in which the nitrogen atom bearing said group Z₁ or Z₂ is free and the other nitrogen atoms have been optionally pre-protected, in a manner known per se, the compound Y′1-Br of formula: 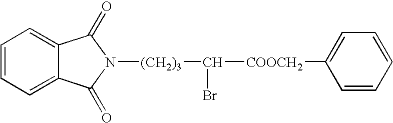
prepared according to Tetrahedron Letters 38(47), 1997, 8253-8256 and J. Org. Chem., 50, 1985, 560-565, whereas, for those in which Z₁ or Z₂ represents: 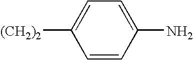
the compound Y″1-Br of formula: 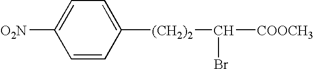
described in J. Org. Chem. 58, 1993, 3869-3876, is reacted.

Next, on the other macrocyclic nitrogen atoms, after optional deprotection thereof, is reacted the brominated diacid, protected in the form of an ester Y′″1Br: 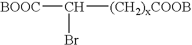
which can, for example, be prepared:
for x=1, B=diphenylmethyl,
   according to J.B.I.C. 4, 1999, 341-347;
for x=2, B═(C₁-C₃)alkyl or benzyl,
   according to WO 00/75241;
and for x=3, B═CH₃,
   according to EP-A-614 899,
      before freeing the amine function of the phthalimido group or reducing the nitro group, previously introduced. The acid functions are deprotected by the action: of a base or of an acid in aqueous or aqueous-alcoholic medium, before or after formation of the amino group.

After the carboxylic acid functions have been freed, the gadolinium complex is then prepared according to one of the methods known in particular from U.S. Pat. No. 5,554,748 or Helv. Chim. Acta, 69, 1986, 2067-2074, by the action of Gd₂O₃ or GdCl₃ in aqueous medium at a pH of between 5 and 7.

During the preparation of product of formula V in which T′ represents pyridyl, when, in the first stage, Y′Br or Y″Br is reacted on the macrocycle in which none of the nitrogen atoms are blocked, the asymmetric derivatives of the following type are obtained, after reaction with Y′″Br and formation of the amino group:

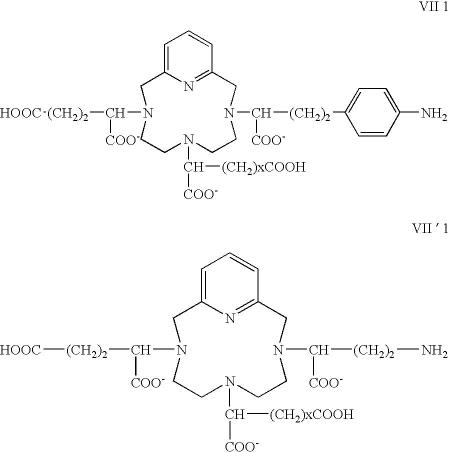

These compounds VII′1 are referred to as HR PCTA of the N-functionalized type, the amine function being located on a side arm.

To obtain the symmetrically substituted derivative, use may be made of the process according to the reaction scheme of Table 1 below, in which x and B have the meanings above, using the protected triamine (a) described in Tetrahedron Letters, 41(39), 2000, 7443-7446, followed by the stages similar to those mentioned for the asymmetric compound:

TABLE 1

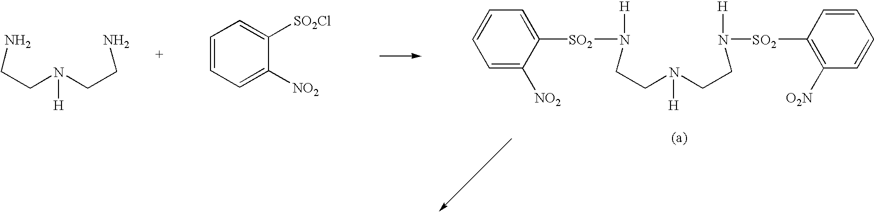

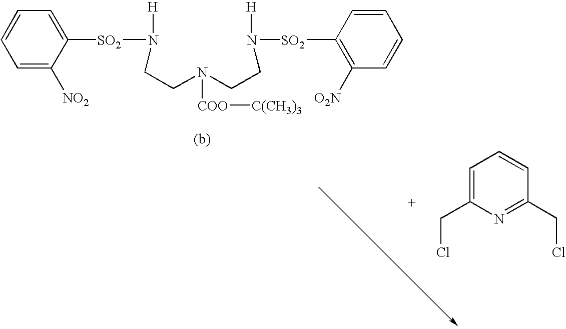

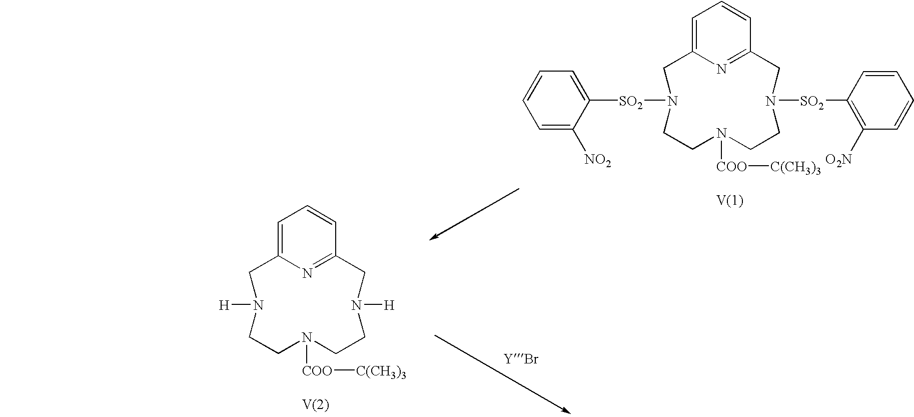

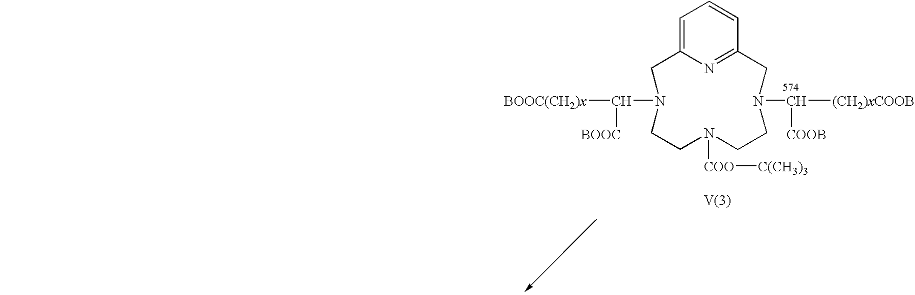

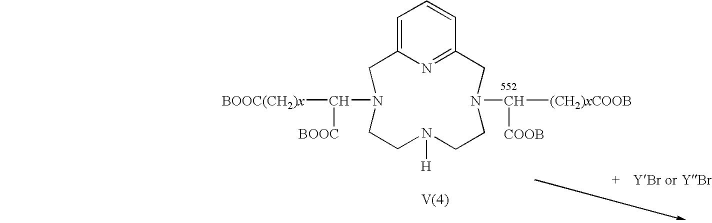

(The compounds V(1) to V(4) are intermediates of V.1).

To prepare the compounds of formula VI 1, a Heck reaction is carried out on the bicyclic macrocycle, brominated on the pyridyl ring, of formula: 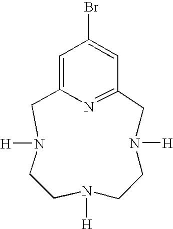
described in J. Heterocyclic Chem. 27, 1990, 167-169, followed by a reduction. The Heck reaction can be carried out under the conditions described in Metal Catalyzed cross-coupling reactions, Ed. F. Diederich, P. J. Stang, Wiley, VCH, chap. 3, p. 99-166. The reaction scheme for the first stages of the process for preparing VI is represented in Table 2; the ester groups are then hydrolyzed and the gadolinium complexed, before or after deprotection of the amino group by the action of trifluoroacetic acid.

To prepare the amides resulting from reaction of the compound VI with an amine RNH₂, it is preferred to carry out the amidation before deprotecting the aliphatic amine.

TABLE 2

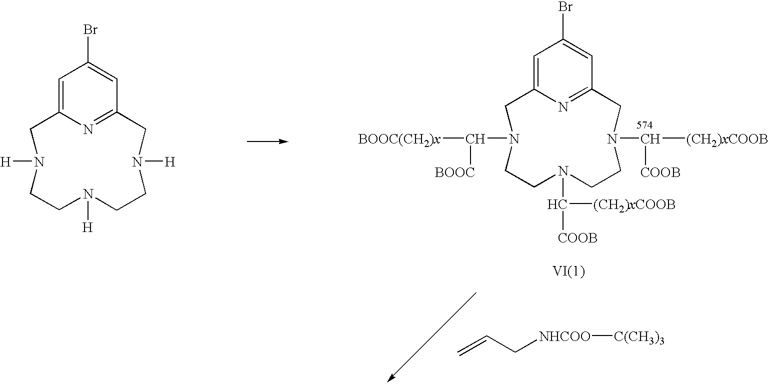

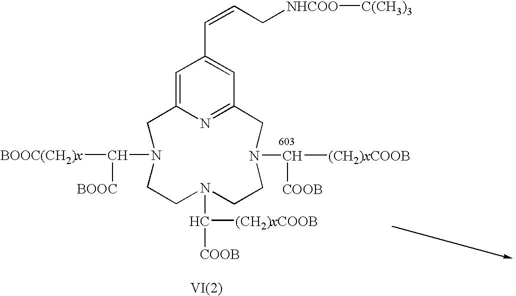

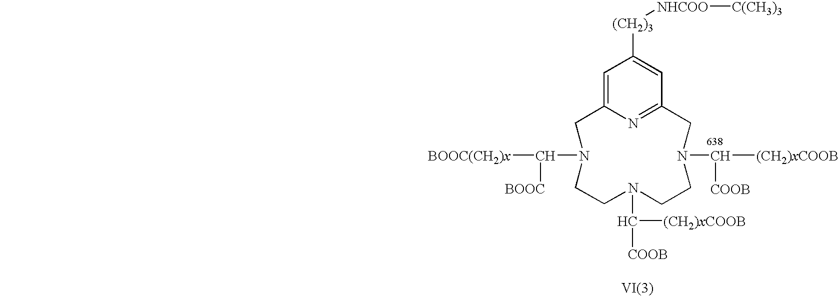

The compounds VI(1) to VI(4) are intermediates of VI 1.

Similarly, the compounds A2 were obtained from precursors A′2NH, which are dimers of formula: 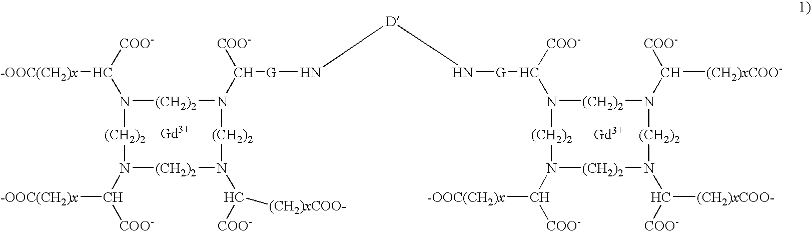
V2 precursor of II′2
in which x=1, 2 or 3, preferably x=2 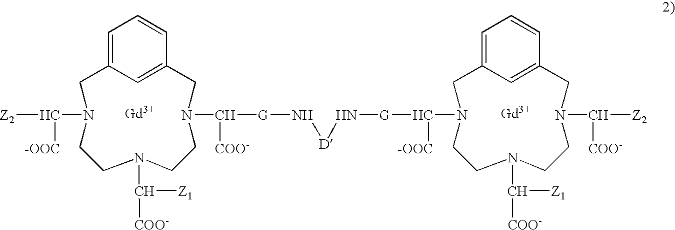
V″a2 precursor of II″a2
   with, for 1) and 2):
G-NH is chosen from the groups —(—CH₂)₃NH or 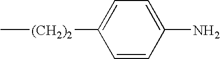
   with, for 2):
Z₁ and Z₂ are (CH₂)ₓCOOH, in which x=1, 2 or 3, preferably x=2 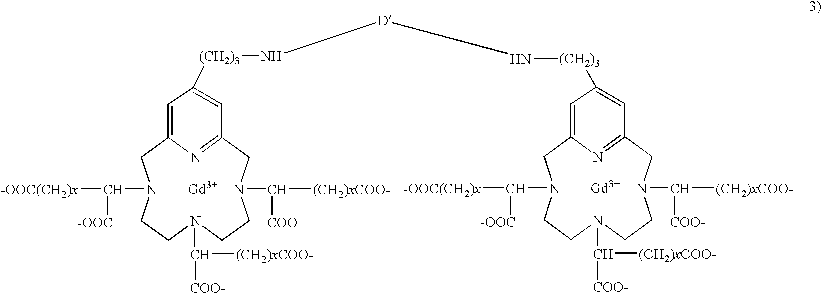
VI 2 precursor of II′″2
with x=1, 2 or 3, preferably x=2

According to one aspect, the invention relates to intermediate compounds for preparing a compound of formula (E), said intermediates having the formula:
L-[(D)_{q}-(I_{a,b,c,d,e,f,g})ᵣ]
in particular the compounds II′2, II″a2, II″b2, II′″2, coupled to a linker L of squarate type. In fact, these intermediates of novel and inventive structure make it possible to produce polymetallic compounds (E) with high relaxivity and good selectivity.

After having described the HR chelate component, the biovector component is now described.

According to a preferred embodiment, the biovector is an agent capable of targeting cellular receptors or tissue components (extracellular matrix, proteases, etc.) chosen from receptors of myocardial cells, of endothelial cells, of epithelial cells, of tumour cells or of immune system cells, and components of the architecture of normal or pathological tissues.

By virtue of the HR chelates used, the relaxivity of which is high (at least 20 to 30 mMol⁻¹Gd⁻¹), the relaxivity of the HR-bivector compounds obtained is also high, including for small biovector molecules. This constitutes a further advantage compared to products of the prior art which, in order to increase the relaxivity, use, as biovectors, proteins coupled to the signal component.

In terms of solubility the properties are also very advantageous. The biovectors used often pose problems of solubility, as is the case, for example, for folic acid and many peptides. On the other hand, the HR-biovector compounds of the inventors have good solubility properties by virtue of the hydrophilic R groups.

Several embodiments associated with various pathological fields are now described.

As regards the cardiovascular field and high-risk atheroma plaque, various biological systems/mechanisms present in the atheroma plaque are preferred targets for the contrast or therapeutic agents according to the invention: the system involving metalloproteases (MMPs), the thrombus system and the annexin V system.

It is known that matrix metalloproteinases (MMPs) or matrixins, are enzymes: which have the property of degrading the protein components of the extracellular matrix. This extracellular matrix which surrounds cells and tissues consists of proteins such as collagen. The MMPs are classified into 3 groups: gelatinases (type IV collagenases), stromelysins and interstitial collagenases.

MMPs are overexpressed in atheroma plaques. In the cardiovascular field, many studies indicate that MMPs are involved in the remodelling of the extracellular matrix in the plaque. With regard to atheroma plaques, at least eight MMPs are overexpressed therein:

| | MMP |
|---|---|
| Enzyme | reference |
| Interstitial collagenase | MMP-1 |
| Gelatinase A | MMP-2 |
| Gelatinase B | MMP-9 |
| Stromelysin-1 | MMP-3 |
| Matrilysin | MMP-7 |
| Macrophage elastase | MMP-12 |
| Collagenase-3 | MMP-13 |
| Membrane-type-MMP MT-MMP-1 | MMP-14 |

MMP1 and MMP3 are particularly described in Johnson J et al., Activation of Matrix-degrading Metalloproteinases by Mast Cell Proteases in Atherosclerotic Plaques, Arterioscl. Thromb. Vasc. Biol. 1998; 18: 1707-1715.

The invention thus relates to HR-BIOVECTOR products in which the biovector is an MMP inhibitor, for applications in cardiovascular fields. According to a preferred implementation, the inhibitor is a derivatives of ilomastat or a peptide as exemplified later.

Preference will also be given to compounds of general formula Iₓ-Lₙ-(ChFR)_{y}, with the MMP inhibitors Iₓ chosen from those described in Current Medicinal Chemistry, 2001, 8, 425-474; Chem. Rev, 1999, 99, 2735-2776. Use may in particular be made of MMP inhibitors referred to as TIMPs, recalled in DDT vol 1, No. 1, January 1996, Elsevier Science, 16-17; Bioconjugate Chem, 2001, 12, 964-971.

For the thrombus, it is known that:
the GpIIb/IIIa receptor is expressed on activated platelets (it is already used in therapeutics as a target for antiplatelet agents);
fibrin is associated with thrombosis.

More precisely, as regards the thrombus, the involvement of GpIIb/IIIa glycoproteins on activated platelets has been demonstrated. The platelets are anuclear fragments of bone marrow megakaryocytes which play a pivotal role in the processes of atherosclerosis and of thrombosis. The most commonly used conventional antiplatelet medicinal products are aspirin, ticlopidine and clopidogel. Knowledge of the molecular mechanisms resulting in platelet aggregation has made it possibile to develop a new family of molecules directed against the platelet receptor for fibrinogen, the integrin GPIIb/IIIa. The major advantage compared to the antagonists mentioned above is that the final step of platelet activation is blocked, independently of the route of activation of the platelets. Since platelet-platelet interaction is critical for formation of the thrombus, binding of fibrinogen (which forms bridges between the platelets) to the GP IIb/IIIa complex is a key event in hemostasis and thrombosis. When the platelets are activated, the GP IIb/IIIa glycoprotein receptors which are at the surface of the platelet membranes undergo a modification of their spatial conformation and can then bind molecules of fibrinogen soluble in the plasma, and calcium. The fibrinogen is linked between the platelets via Ca²⁺-fibrinogen bonds forming a network in which the blood cells will be trapped. The thrombin, by converting the fibrinogen to fibrin, tightens the mesh of this net. This aggregation will lead to the formation of a thrombus in the damaged: area. Many studies have therefore been carried out in order to identify, on the GP IIb/IIIa receptor, the ligand interaction sites. The GP IIb/IIIa complex is an important membrane-bound heterodimeric glycoprotein complex in platelets (approximately 50 000 copies per platelet).

At least two series of peptides, corresponding to amino acid sequences naturally present in human fibrinogen, are known to inhibit the binding of adhesion macromolecules to the GPIIb/IIIa receptor: the sequence Arg-Gly-Asp (RGD) and the Lys-Gln-Ala-Gly-Asp-Val gamma chain.

The sequence Arg-Gly-Asp (RGD) was initially identified as the adhesion sequence of fibronectin, an integrin which plays an important role in platelet-platelet and platelet-vessel interactions after its release by platelets. This sequence is also present in fibrinogen, von Willebrand factor and vitronectin (role in fibrinolysis and binding to vessels).

The GPIIb/IIIa complex recognizes this sequence which inhibits the binding of fibronectin, of fibrinogen, of von Willebrand factor and vitronectin on platelets. All these ligands contain at least one RGD sequence; while fibrinogen contains two thereof per half-molecule. In vivo, fibrinogen is the main ligand due to its high concentration in the plasma. The invention thus relates to:
HR-BIOVECTOR products in which the biovector is capable of targeting the GpIIbIIIa receptor;
HR-BIOVECTOR products in which the biovector is capable of targeting fibrin, in particular with peptides, selected for fibrin monomers in order to differentiate fibrin from the soluble fibrinogen molecule.

The invention relates in particular to HR-BIOVECTOR products in which the biovector comprises an RGD motif, for cardiovascular applications.

Use may, for example, be made of peptides described in WO 2001/9188, Seminars in nuclear medicine, 1990, 52-67, Nucear Medicine and Radiology, 28, 2001, 515-526, the apcitide Acutect from the company Diatide.

As regards the field of oncology, the specific imaging obtained by virtue of the present invention is aimed at obtaining, according to one embodiment, labelling of neoangiogenesis, specific targeting of tumour cells or alterations in the extracellular matrix, not obtained with known techniques. Various biological systems (or mechanisms) associated with tumour development are preferred targets for the contrast or therapeutic agents according to the inveniton: the system involving compounds capable of binding to folate receptors, the system involving MMPs, the system involving growth factors involved in angiogenesis.

It is known that folates play an essential role in the biosynthesis of purine and pyrimidine bases in all living organisms. They are thus involved in the processes of cell proliferation involving various enzymes using folates as cofactors or as substrates. Their metabolism is involved, firstly, in the synthesis of the pteridine ring and, secondly, in reactions to functionally modify, to oxidize or to reduce the already formed pteridine ring. The cellular uptake of endogenous folates, like antifolates, can be regulated by 2 transport proteins:
Folate Binding Protein (FBP), also called Folate Receptor
Reduced Folate Carrier (RFC)

U.S. Pat. No. 6,221,334 recalls the involvement of folates in this field, and describes compounds associating folic acid or methotrexate with a chelate.

Application WO 02/087424 describes compounds associating folates with a low-relaxivity chelate, these folates being necessarily devoid of natural amino acids, which is not necessarily the case of the folate biovectors of the present invention.

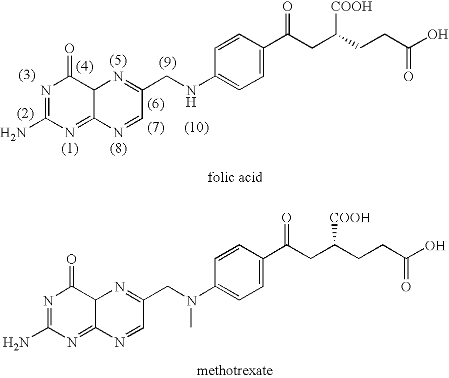

According to one embodiment, the invention relates to HR-BIOVECTOR products in which the biovector is a derivative capable of targeting a folate receptor, this biovector being capable of giving rise to specific recognition of tumour cells, and being coupled to an immobilized chelate HR Ch.

The invention relates in particular to the compounds (E) which are written: 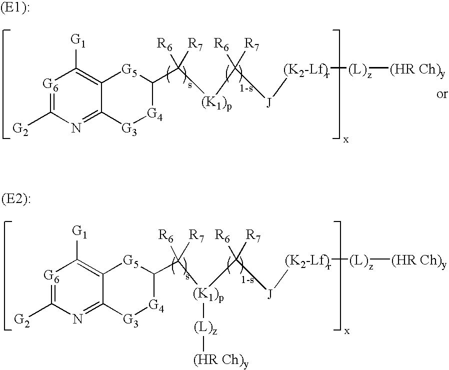
in which:

a) G1 is chosen independently from the group consisting of: halo, R_{f}2, OR_{f}2, SR_{f}3, NR_{f}4R_{f}5; preferably G1 is NH2 or OH

b) G2 is chosen independently from the group consisting of: halo, R_{f}2, OR_{f}2, SR_{f}3, and NR_{f}4 R_{f}5;

c) G3, G4 represent divalent groups chosen independently from the group consisting of —(R_{f}6′)C═, —N═, —(R_{f}6′)C(R_{f}7′)-, —N(R_{f}4′)-;

preferably, G3 is —N═ (folic acid) or —CH— (compounds described later: CB3717, raltitrexed, MAI) when the ring comprising G3 is aromatic, and G3 is —NH— or —CH₂— (compounds described later: AG-2034, lometrexol) when the ring comprising G3 is non-aromatic;

preferably, G4 is —CH— or —C(CH₃)— when the ring comprising G3 is aromatic, and —CH₂— or —CH(CH₃)— when the ring comprising G3 is non-aromatic;

f) G5 is absent (compound pemetrexed) or chosen from —(R_{f}6′)C═, —N═, —(R_{f}6′)C(R_{f}7′)-, —N(R_{f}4′)—;

g) the ring J is a8 possibly heterocyclic aromatic 5- or 6-membered ring, it being possible for the atoms of the ring to be C, N, O, S;

h) G6 is N or C (compound described later: 3-deaza-ICI-198,583)

i) K1 and K2 are chosen independently from the group consisting of —C(Z_{f})-, —C(Z_{f})O—, —OC(Z_{f})-, —N(R_{f}4″)-, —C(Z_{f})-N(R_{f}4), —N(R_{f}4″)—C(Z_{f}), —O—C(Z)—N(R_{f}4″)—C(Z_{f})-O—, N(R_{f}4″)—C(Z_{f})-N(R_{f}5″)-, —O—, —S(O)—, —S(O)2-, —N(R_{f}4″)S(O)2—, —C(R_{f}6″)(R_{f}7″)-, —N(C≡CH)—, —N(CH₂—C≡CH)—, C₁-C₁₂ alkyl and C₁-C₁₂ alkoxy; in which Zf is O or S; preferably, K1 is —N(R_{f}4″)- or —C(R_{f}6″)(R_{f}7″)- with R_{f}″4, R_{f}6″, R_{f}7″ being H; A2 possibly being covalently bonded to an amino acid;

j) R_{f}1 is chosen from the group consisting of: H, halo, C₁-C₁₂ alkyl and C₁-C₁₂ alkoxy; R_{f}2, R_{f}3, R_{f}4, R_{f}4′, R_{f}4″, R_{f}5, R_{f}5′″, R_{f}6″ and R_{f}7″ are chosen independently from the group consisting of: H, halo, C₁-C₁₂ alkyl, C₁-C₁₂ alkoxy, C₁-C₁₂ alkanoyl, C₁C₁₂ alkenyl, C₁-C₁₂ alkynyl, (C₁-C₁₂ alkoxy)carbonyl and (C₁C₁₂ alkylamino) carbonyl;

h) R_{f}6 and R_{f}7′ are chosen independently from the group consisting of: H, halo, C₁-C₁₂ alkyl, C₁-C₁₂ alkoxy; or R_{f}6 and R_{f}7 together form O═;

i) R_{f}6′ and R_{f}7′ are chosen independently from the group consisting of: H, halo, C₁-C₁₂ alkyl, C₁-C₁₂ alkoxy; or R_{f}6′ and R_{f}7′ together form O═;

j) Lf is a divalent linker which includes, where appropriate, a natural amino acid or a natural poly(amino acid), bonded to K2 or to K1 via its alpha-amino group via an amide bond;

k) n, p, r and s are independently 0 or 1.

The formula (E) includes the tautomeric forms, for example compounds for which G1 is OH, SH or NH.

For the compounds of the invention in which at least one of the groups K1, K2,

R_{f}1, R_{f}2, R_{f}3, R_{f}4, R_{f}4′, R_{f}4″, R_{f}5, R_{f}5″, R_{f}6, R_{f}7″, R_{f}6, R_{f}7, R_{f}6′ and R_{f}7′ comprises an alkyl, alkoxy, alkylamino, alkanoyl, alkenyl, alkynyl, alkbxycarbonyle or alkylaminocarbonyl group, the group preferably comprises 1 to 6 carbon atoms (C₁-C₆), more preferably 1 to 4 carbon atoms (C₁-C₄).

Among the compounds stated above, the inventors have in particular focused on the derivatives:

a)

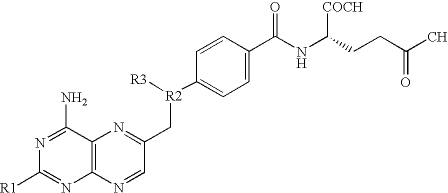

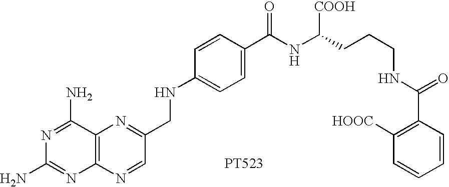

| | R1 | R2 | R3 |
|---|---|---|---|
| MTX | NH₂ | N | CH₃ |
| 2-dMTX | H | N | CH₃ |
| 2-CH₃-MTX | CH₃ | N | CH₃ |
| AMT | NH₂ | N | H |
| 2-dAMT | H | N | H |
| 2-CH₃-AMT | CH₃ | N | H |
| Edatrexate | NH₂ | C | C₂H₅ |

b)

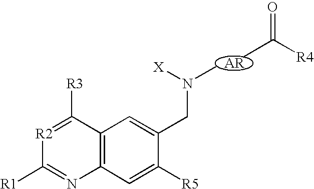

| | R1 | R2 | R3 | R4 | R5 |
|---|---|---|---|---|---|
| X = propargyl | | | | | |
| CB3717 | NH₂ | N | OH | Glu | H |
| ICI-198.583 | CH₃ | N | OH | Glu | H |
| 3-deaza-ICI-198.583 | CH₃ | CH | OH | Glu | H |
| 4-H-ICI-198.583 | CH₃ | N | H | Glu | H |
| 4-OCH₃-ICI-198.583 | CH₃ | N | OCH₃ | Glu | H |
| Glu → Val-ICI-198.583 | CH₃ | N | OH | Valine | H |
| Glu → Sub-ICI-198.584 | CH₃ | N | OH | Suberate | H |
| 7-CH₃-198.583 | CH₃ | N | OH | Glu | CH₃ |
| X = methyl | | | | | |
| raltitrexed | CH₃ | N | OH | Glu | H |
| 2-NH₂-ZD1694 | NH₂ | N | OH | Glu | H |

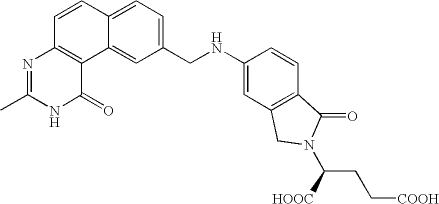 BW1843U89

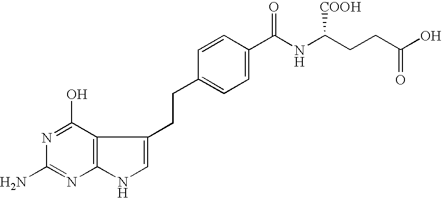

Pemetrexed

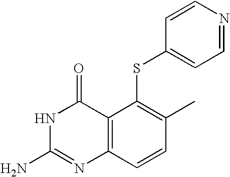

AG337

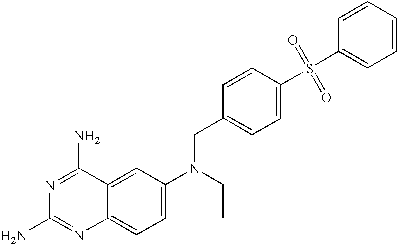

AG377

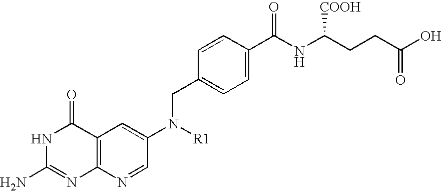

| | R1 |
|---|---|
| 5-d(i)PteGlu | H |
| N⁹—CH₃-5-d(i)PteGlu | CH₃ |
| N⁹—CHO-5-d(i)PteGlu | CHO |

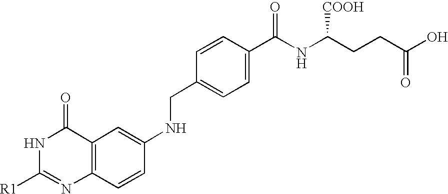

| | R1 |
|---|---|
| IAHQ | NH₂ |
| 2-dIAHQ | H |
| 2-CH₃-dIAHQ | CH₃ |

The invention preferably relates to the compounds (E) using the following biovectors B:

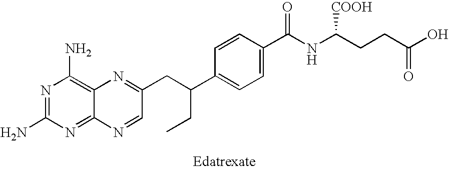

By substituting the N¹⁰ of MTX with a carbon bearing the ethyl group, the cellular uptake via RFC and also the polyglutamatation are greater for tumour cells/normal cells.

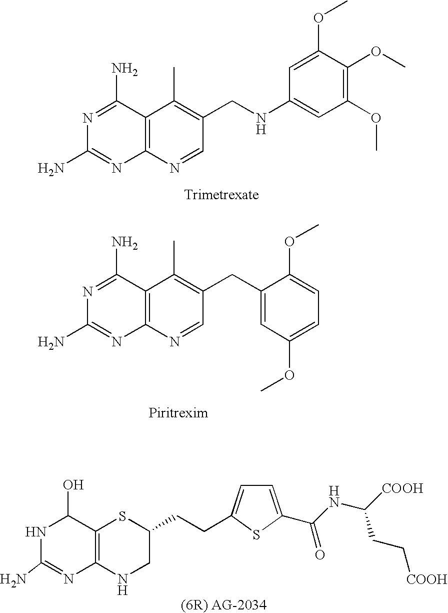

The sulphur in the 5-position makes it possible to obtain better filling of the hydrophobic pocket of GARFT than methylene (Kd/FBP=3.2 pM).

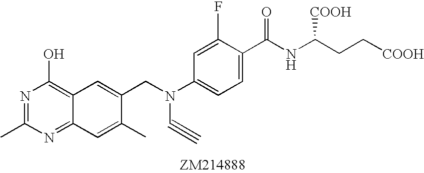

This structure is, like that of all the compounds studied by the inventors which are substituted on the nitrogen in the 10-position, very different from folic acid.

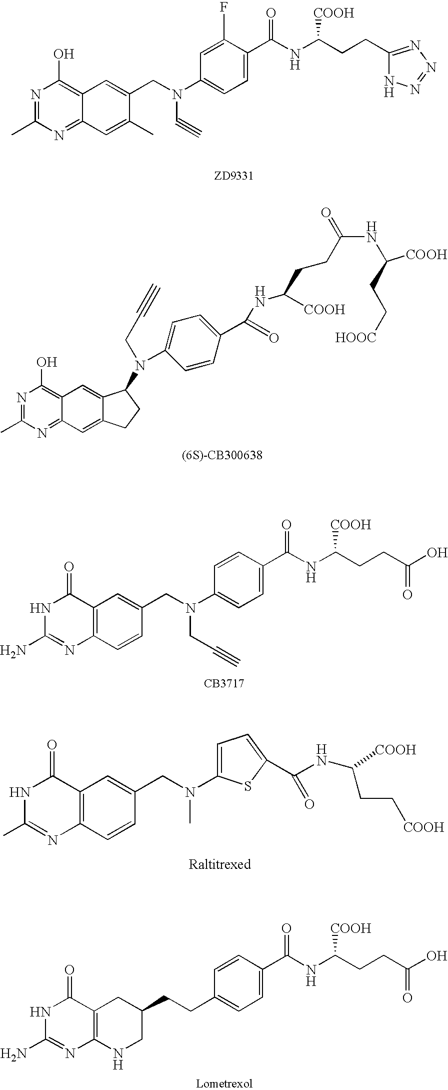

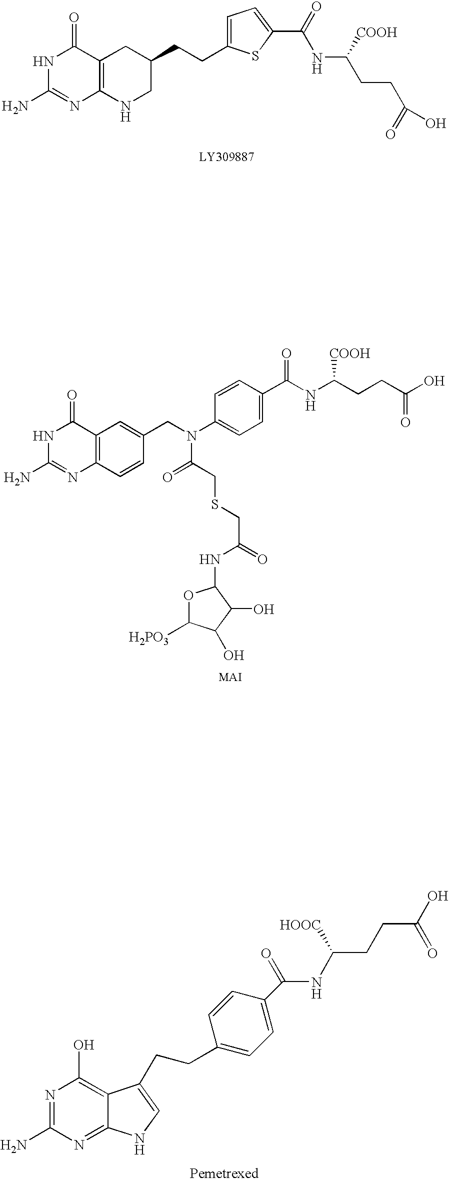

These structures, which do not contain the pteroic backbone, are very different from that of folic acid from a structural point of view.

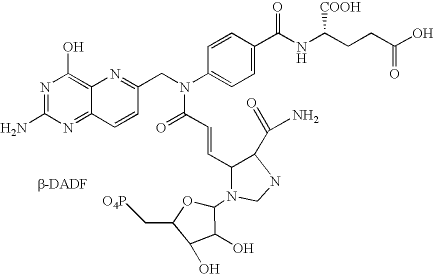

By virtue of activated structure analysis, the inventors have identified several structures favourable to interaction with RFC (the affinity increases when the amine in the 2-position is replaced with a methyl, the nitrogens in the 5-position and 10-position can be replaced with carbons; the pyrazine ring can be tetrahydrogenated or replaced with a pyrrole (pemetrexed)), and several structures favourable to interaction with FBP (4 inhibitors of thymidilate synthetase TS have an affinity greater than folic acid: pemetrexed>CB3717>IAHQ>2-NH2-ZD1694; the affinity is conserved for the 5,8-dideazaisofolic derivatives; the pyrazine ring can be tetrahydrogenated, replaced with a benzene (CB3717) or replaced with a pyrrole (pemetrexed); replacement of the glutamic acid is possible).

In the case of the folates or derivatives, when Lf contains an amino acid, the compound (E1) has a carboxylic acid function in the alpha position and a carboxylic function in the gamma position.

a) With a compound of formula (E1), it is thus possible to choose to graft, for example:
a single HR Ch, as in the compound BIO-FOLATE.I with z=1, x=1, y=1, the group ChRR being in the form of a dimer comprising two chelates linked via a divider, the group [L-HR Ch] being grafted in the gamma-position;
or two groups HR Ch, as in the compound BIO-FOLATE.II with z=2, x=1, y-2, the groups HR Ch being in the form of a dimer, one of the groups [L-HR Ch] being grafted in the gamma-position, the other in the alpha-position, the compound BIO-FOLATE.II therefore being a tetramer.

b) With a compound of formula (E2), when Lf contains an amino acid, it is thus possible to choose to graft onto the K1 in the 10-position and, optionally, also as for (E1) in (a).

As regards MMPs in the oncology field, it is known that MMPs have two distinct functions:

a) they contribute to tumour dissemination by destroying the extracellular matrix;

b) they create an environment which promotes the growth and the angiogenesis of primary tumours and metastasized tumours.

The MMPs expressed in the main human tumours are in particular the following:
breast cancer: MMP-1,2,3,7,9,11,13,14;
colorectal cancer MMP-1,2,3,7,9,11;
lung cancer MMP-2,3,7,9,11,14;
prostate cancer: MMP-1,2,3,7,9.

There is a frequent correlation between the state of tumour progression and the level of expression of MMPs. In general, malignant tumours express a greater amount of MMP than benign tumours.

The invention thus relates to HR-BIOVECTOR products in which the biovector is an MMP inhibitor, for applications in the oncology field. According to preferred embodiments, use will be made of inhibitors selected from those described in Current Medicinal Chemistry, 2001, 8, 425-474; Chem. Rev, 1999, 99; 2735-2776. Use may in particular be made of MMP inhibitors referred to as TIMPs, recalled in DDT vol. 1, No. 1, January 1996, Elsevier Science, 16-17; Bioconjugate. Chem, 2001, 12, 964-971.

As regards angiogenesis, studies have shown that angiogenesis is a prognostic factor in diverse tumours, in particular breast cancer, kidney cancer, prostate cancer, colon cancer and brain cancer, and also melanoma.

The invention thus relates to HR-BIOVECTOR products in which the biovector is capable of targeting an angiogenesis marker.

It has been shown that certain endothelial growth factors are tumour specific. Endothelial cells, which constitute the inner vessel wall, show no natural tendency to proliferate in normal adults. On the other hand, in pathological situations, for example during the development of tumours, or the formation of metastases, the increased needs in oxygen and in nutritive supplies are transported by an increase in local irrigation. The tumours thus derive, to their benefit, a new vascular network, a process known as angiogenesis.

Vascular endothelial cell growth factor (VEGF) is a powerful and selective angiogenic growth factor. It acts by stimulating at least three receptors on the extracellular membrane: VEGFR-1 (Flt-1), VEGFR-2 (Flk-1/KDR) and NP-1. VEGF receptors belong to the large RTK (tyrosine-kinase receptor) family. These proteins of the integrin family have an extracellular region capable of binding ligands, a transmembrane domain and a cytoplasmic region carrying the tyrosine kinase activity. In the case of VEGFRs, the kinase domain is interrupted by a short sequence specific to each receptor.

The invention thus relates to HR-BIOVECTOR products in which the biovector is an agent capable of binding to angiogenic receptors present at the surface of endothelial cells.

Use may in particular be made of biovectors (in particular peptides obtained by phage display) described in documents WO 01/012809, WO 01/83693, WO 02/057299 (peptides of 8 amino acids targeting VEGFR3); Nuclear Medicine Communications (1999), 20, Pharmacological Review, 179, 206, 2000; Journal of Biological Chemistry, 2000, 275, 13588-13596; Journal of Biological Chemistry, 2002, 277, 45, 43137-43142; J. Mol. Biol, 2001, 316, 769-787; Biochemistry, 1998, 37, 17754-17772; Chem. J. Biochem. Mol. Biol, 2000, 16, 803-806. Use may in particular be made of (i) inhibitors of the VEGF-related enzyme activity, such as quinazoline, aminothiazole or anthranilamide compounds, (ii) compounds which are VEGF antagonists, in particular small molecules, such as dibenzothiophenes and molecules of documents JP2001-353075, JP2000-395413, antibodies.

It is also known that the integrin αvβ3 is hardly expressed in the vascular system, but is overexpressed in tumour cells (final-stage glioblastoma, ovarian carcinoma melanoma). αvβ3 takes part in angiogenesis at various stages: αvβ3 regulates endothelial cell adhesion to the matrix, it transmits signals to the cell nuclei and is a receptor which is pro-angiogenic by cooperating with the endothelial cell growth factor receptor (VEGFR-2, flk). αvβ3, acting with membrane-type metalloproteinase-1 (MT1-MMP), is responsible for the activation of the metalloproteinase-2 of the matrix at the surface of the cells. The blocking of this receptor, but also of αvβ5, with RGD peptides or with antibodies induces apoptosis of the cells. αvβ3, like αvβ5, is involved especially in the proliferative phase of angiogenesis. Many matrix proteins have a common sequence: Arg-Gly-Asp (referred to as RGD), which has been identified as the site of interaction with certain integrins. A large number of antiogenesis inhibitors comprising this RGD sequence have thus been developed.

The invention thus relates to HR-BIOVECTOR products in which the biovector is an RGD peptide suitable for applications in oncology.

For the RGD peptides targeting αvβ3, the inventors prefer biovectors for inhibiting angiogenesis exhibiting a high affinity for αvβ3 (in order to prevent binding of matrix proteins) but a low affinity for αIIbβ3. In fact, it has been shown that αIIbβ33 antagonists can cause adverse bleeding problems. The inventors prefer in particular antagonists having a cyclic RGD peptide sequence which is more stable to enzyme degradation and better affinity and selectivity, the conformation of the peptide being maintained in a favored position due to rotation restriction.

More precisely, after a structure-activity analysis of the RGD peptides, the inventors prefer most particularly, in order to be under conditions favorable to maintaining affinity for αvβ3, cyclic RGD peptides in order to prevent degradation by enzymes (exopeptidase and endopeptidase), with quite a short ring in order for it to be more rigid (5-amino acid ring preferable to a 6-amino acid ring), with an amino acid in the D configuration. The distance between the β-carbons of aspartic acid and of arginine is typically less than 6.6 Å in order to have greater selectivity for αvβ3 than for αIIbβ3 (the site of which is larger than that of αvβ3). A hydrophobic amino acid close to the aspartic acid also promotes better selectivity. Such a structure is optimal for correct exposure of the hydrophobic and hydrophilic functions in the receptor site. The inventors prefer in particular the peptide cyclo(Arg-Gly-Asp-Dphe-Val), also referred to as cyclo(RGDfV), the lower-case letters indicate a D configuration for the corresponding amino acid. It exhibits an IC₅₀ of nanomolar (2-2.5 nM) order. This peptide is particularly advantageous for the side amine function of lysine which can react with an HR derivative. The synthesis thereof is described in X. Dai, Z. Su, J. O. Liu; Tetrahedron Letters (2000), 41, pp 6295-6298. 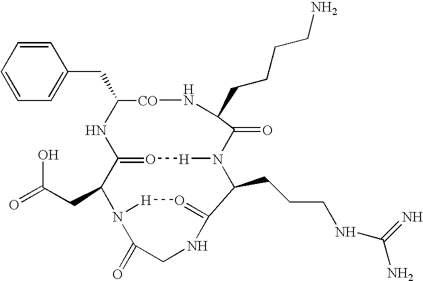
RGDfK Peptide

It permits coupling to functionalized HR Gd chelates, which are in particular COOH—, squarate- or isothiocyanate-functionalized. The inventors have had to overcome the technical difficulties associated with the choice of protection of the amino acids, and with the choice of the coupling medium (solid with questions of solubility of the Gd ligands or in solution).

Use may also be made of compounds having a conformation with restricted flexibility, these peptides having good affinity and selectivity for αvβ3:
peptides in which the RGD motif is placed between two cysteines, the activity for I′αvβ3 increasing, for example (cyclo(CRGDC));
peptides in which the RGD group is not in the ring, but is bordered by two rings, at least one of which was formed by a disulphide bridge between 2 cysteines (WO02/26776)

Use may also be made of:
polypeptide biovectors chosen such that they interact in vivo with at least one enzyme, in particular MMP, this interaction resulting in stronger binding with a target protein and in an increase in relaxivity, as described in document WO 01/52906;
biovectors comprising an enzymatic cleavage site, the cleavage resulting in a conformational modification and exchanges of water molecules at the level of the HR chelate (principle described in U.S. Pat. No. 5,707,605 with non-HR chelates);
biovectors derived from antibodies, such as LM609 (Nature Medicine, vol 4, 5, May 1998, 623).

The invention also relates to HR-BIOVECTOR products in which the biovector is a peptidomimetic of the RGD peptide, suitable for applications in oncology. Use may be made of:
peptides with substitution of one or two peptide bonds by thioamide bonds (CSNH) or by keto methylene groups (COCH₂) provided that the substitutions do not induce any important conformation change;
peptides (EMD 121974, also called Cilengitide: cyclo(RGDf-N(Me)V), which exhibit an N-methylation of each of the amino acids of the cyclic peptide (RGDfV), this peptide exhibiting very high affinity (IC₅₀=0.58 nM) and very great selectivity (1500 times greater than for αIIbβ3); 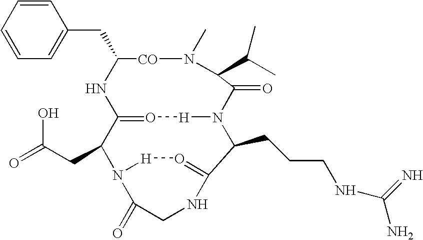
   peptides having the following sequence: cyclo(Xaa-Yaa-GD-Mamb), the Mamb group being N-aminomethylbenzoic acid. It is demonstrated that DXaa-N-MeArg peptides are very active antagonists of αIIbβ3, whereas LXaa-Arg antagonists are selective for αvβ3; 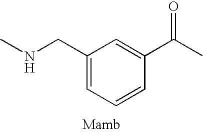
the cyclic peptide (RGDD(tBuG)Mamb) exhibiting very high affinity for αvβ3 (IC₅₀=0.6 nM versus 14 μm for αIIbβ3) due to the hydrophobic nature of the amino acid neighboring the aspartic acid and to the Mamb group which makes the peptide less flexible in aqueous solution;
the peptide XJ735 (DUPONT) having the group: cyclo(ARGD-Mamb). This makes it possible to inhibit the binding of fibrinogen to the αvβ3 receptor (IC₅₀₌₇₀ nM) but does not block other integrins (αvβ5, α5β1);
peptides obtained by introducing various groups in place of Dphe-Val (or fV) into the reference peptide RGDfV.

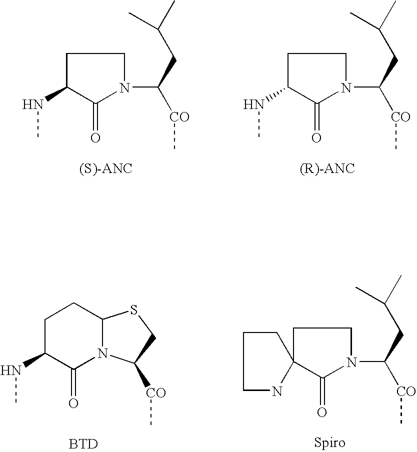

These groups were synthesized in order to mimic the βII′ folding, but also to reduce the flexibility in the β-rotation region. The most active agent is the cyclo(RGD-(R)-ANC) (IC₅₀=0.8 nM). It is also possible to introduce therein sugars X in place of these two amino acids (fv) in the sequence cyclo (RGDX) (Lohof E. et al; Angew. Chem. Int. Ed. Engl. (2000), 39 (15), pp. 2761-2764);
peptides with introduction of a double bond into the ring (Kawaguchi, et al, Biochemical and Biophysical Research Communications (2001), 288(3), pp. 711-717); 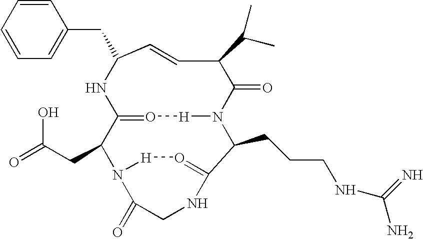
peptides described in Chemlibrary.bri.nrc.ca.

The invention also relates to HR-BIOVECTOR products in which the biovector is a non-peptide molecule which targets the integrin αvβ3, suitable for applications in oncology. Use may be made of the compounds in the following table, the nanomolar affinity of which has been demonstrated, the HR component permitting a signal which is truly enhanced (the diagnostic effectiveness being validated by a screening test such as that used for the RGDfV peptide, details of which are given later).

Compound
Benzodiazepine - benzozepine and derivatives

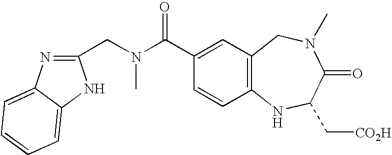

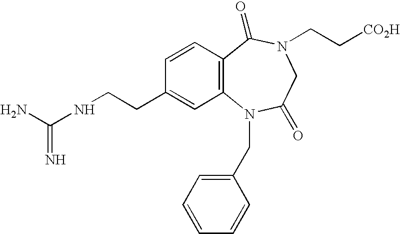

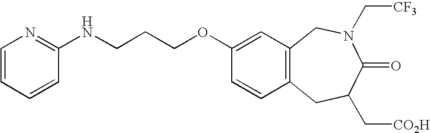

Benzamide

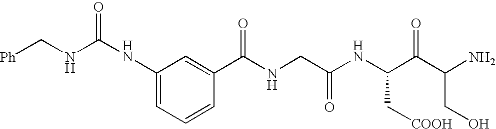

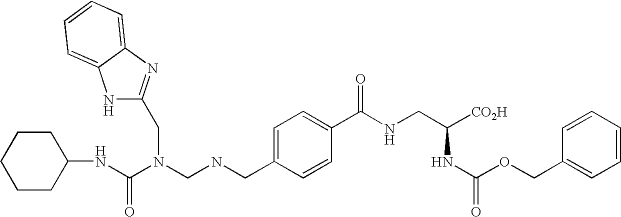

SCH221153

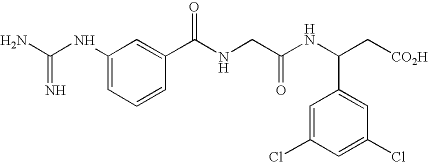

SC 68448
and other compounds of document WO 01/97861

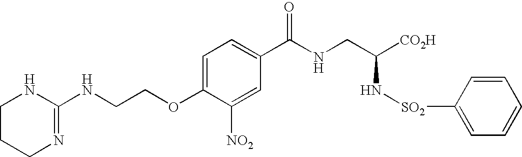

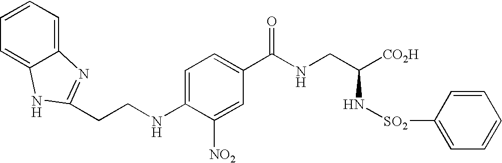

acylpyridine

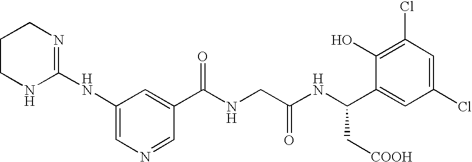

and other compounds of document WO 9952896
Heteropentacycle core

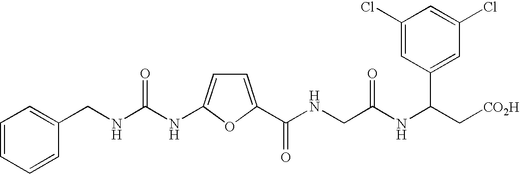

and other compounds of document Lohof et al,
Angew. Chem. Int. Ed. Eng 2000, 39(15), pp 2761-2764

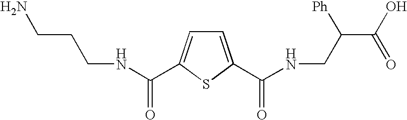

and other compounds based on acetylthiophene of document WO 0000486,

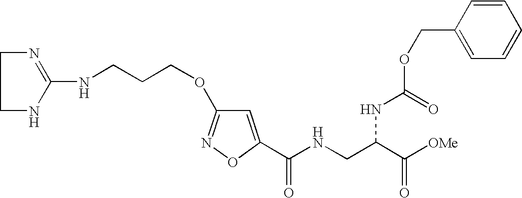

SG 545

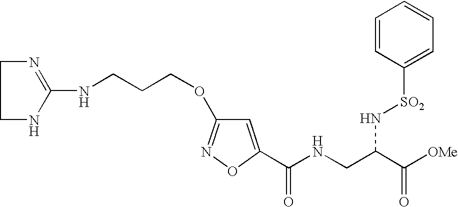

XT 199
(and the compounds SG256, SM256 and XJ735 from Dupont Pharmaceuticals)

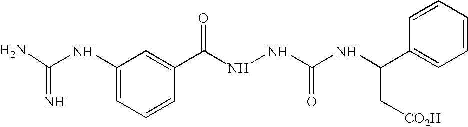

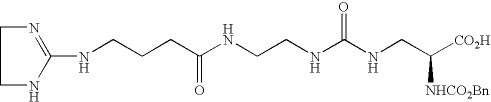

and analogues from document WO 0003973

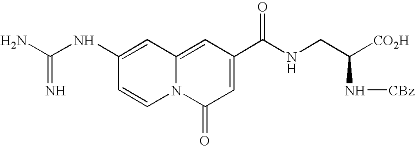

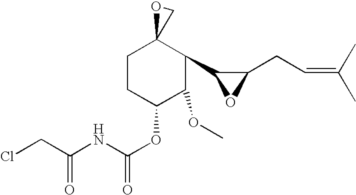

Carbohydrate

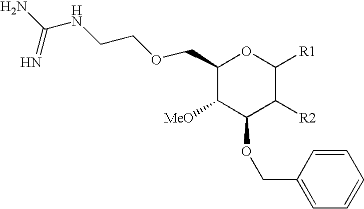

Thiolutin

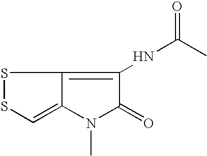

and other compounds with a pyrrothine group

Use may be made of the biovectors mentioned in documents U.S. Pat. No. 6,531,520 (biovectors targeting αvβ3 overexpressed during angiogenesis) and WO 01/198294 (indazole core).

In certain cases, use may be made of several different biovectors in the same compound in order to increase the chances of attaining the same target, for example an RGD peptide and benzodiazepine for αvβ3.

The principle for synthesizing the HR-chelate biovectors with MMP inhibitors may be that used in document WO 01/60416 (pages 91-97) with non-HR-chelate-biovectors (in which the biovector is denoted. Q and the linker Ln).

In addition to the biovectors of the RGD type or functional equivalents mentioned above, use may be made of the following MMP-inhibiting compounds, in the knowledge that the detailed description gives experimental protocols which make it possible to finalize an in virtro or in vivo screening:
peptides, peptidomimetics, functionally equivalent non-peptides, which are effective in diagnostic or therapeutic terms, selected from commercially available inhibitors, in particular in the 2002 catalogues of Bachem, Amersham;
inhibitors described in documents WO 01/60416, WO 01/60820, WO 2001/92244, EP 558 635, EP 663 823;
inhibitors of the type such as hydroxamates, pyrrolidine hydroxamates, bicyclic hydroxamates, cyclobutyl hydroxamates, succinyl hydroxamates, sulphonamide hydroxamates, alanine hydroxamates, as described in documents EP 793 641, EP 766 665, EP 740 655, EP 689 538, EP 575 844, EP 634 998, WO 99/29667, EP 965 592, EP 922 702, WO 99/52889, WO 99/42443, WO 01/60416 (Dupont; in particular of formulae Ia and Ib);
phosphinic acid-based inhibitors as described in documents EP 725 075, U.S. Pat. No. 5,679,700, WO 98/03516, EP 716 086, WO 2000 74681, WO 2000 04030;
cyclic imide-based inhibitors (U.S. Pat. No. 5,854,275);
tricyclic sulphonamide-based inhibitors (WO 2000 06561);
oxobutyric acid-based inhibitors;
derivatives of TIMPS (Bioconjugate Chem, 2001, 12, 904-971).

Still in the oncology field, HR-BIOVECTOR compounds in which the biovector is based on phosphonates or on bisphosphonates are very useful for cancer of the bone tissues. These compounds are also useful for diseases related to bone problems, due to problems of immunity (autoimmune diseases such as rheumatoid arthritis), to metabolic diseases (osteoporosis etc.) and infectious diseases.

In these compounds, the biovector may have the formula (PO₃H₂)n, n being typically between 1 and 6; with [(BIOVECTOR)-L] for example chosen as in document WO 02/062398. Use may also be made of a bisphosphonate described in U.S. Pat. No. 6,534,488 or U.S. Pat. No. 6,509,324, a commercially available bisphosphonate such as etidronate, clodronate, pamidronatet, alendronate, ibandonate, YH 592, EB-1053 and the like.

The inventors have in particular prepared products of formula:

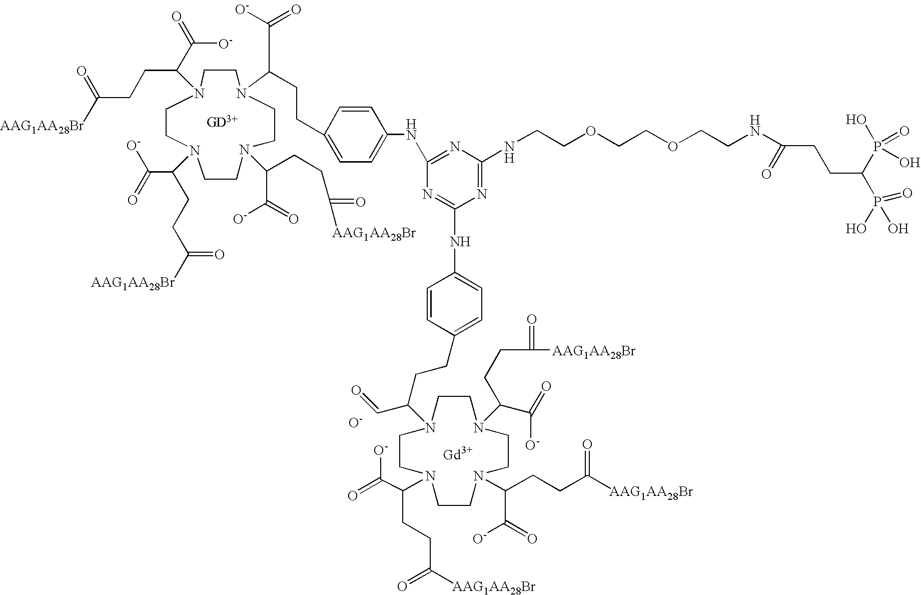

As regards the field of inflammatory and degenerative diseases, the invention is in particular directed towards HR biovectors which target receptors located on macrophages, such as SRA receptors (scavenger receptors), or Fc receptors (US 2002/58284).

The progression of atherosclerosis involves the capture of LDLs and then the oxidation thereof in plaques, The phagocytosis of these oxidized LDLs by macrophages is mediated by a set of receptors referred to as scavenger receptors (SRs). This family of membrane-bound proteins therefore plays a major role in the gradual conversion of macrophages to foam cells. SRs are membrane-bound surface proteins capable of binding senescent cells and also chemically or biologically modified lipoproteins. The main SR groups are classified into various classes:

1/ class A SRs: type I, type II and MARCO

2/ class B SRs: type I, type II and CD36

3/ class D SRs: CD68

4/ class E and F SRs, “lectin-like”: LOX-1

5/ recent unclassified SRs: SR-PSOX.

As recalled in document US A 2002/0127181 and in De Winther et al., ATVB 2000; 20: 290-297; Kunjathoor et al., J Biol. Chem. 2002; 277: 49982-49988, the SRA receptor is overexpressed by macrophages in cardiovascular diseases (atherosclerosis, atheroma plaque, coronary artery disease, thrombosis, ischemia, myocardial infarction etc.). The use of products according to the invention for targeting SRA associated with these pathologies is part of the invention. For example, an SRA antagonist will be used as a biovector, with a superparamagnetic metal for an MRI study or a radioisotope which can be used in scintigraphy or in positron emission tomography (PET and derived techniques).

Among the biovectors usefeul fror SRA targeting, short peptide ligand specific for SR-AI are of formula
X₁—X₂-Arg-Phe-Leu-Arg-Cys-Trp-Ser-Asp-X₃-Pro-X₄
hereinafter also referred to as
X₁—X₂-RFLRCWSD-X₃—P—X₄
in which

X₁ is optionally present and may be a carboxylic acid; or one or more D- or L-amino acids or analogues or mimetics thereof; or an anti-inflammatory drug; or a medicament for treating atherosclerosis; or a detectable label. The carboxylic acid may be a synthetic or natural carboxylic acid such as the C-10 to C-18 fatty acids such as citric acid, glycolic acid, tartaric acid or lactic acid; an unsaturated acid, such as caproic acid or oleic acid; a branched carboxylic acid such as naphthenic acid of molecular weight of from 200 to 500; an aromatic carboxylic acid such as benzoic or salicylic acid, or any other carboxylic acid;

X₂ is a D- or L-amino acid or a peptide, preferably X₂ is selected from the group consisting of the peptide (Leu or Ala or Ile)-Ser-(Leu or Ala or Ile)-(Glu or Asp), the peptide Ser-(Leu or Ala or Ile)-(Glu or Asp), the dipeptide (Leu or Ala or Ile)-(Glu or Asp), and the amino acids Glu and Asp, wherein the symbols indicate the amino acid in line with IUPAC nomenclature and symbolism for amino acids and peptides;

X₃ is a D- or L-amino acid, preferably selected from Ala, Ser, Leu, Ile, Cys and Thr, more preferably selected from Ala and Ser;

X₄ is optionally present and may be a D- or L-amino acid, preferably selected from Ala, Ser, Leu, Ile, Cys and Thr, more preferably selected from Ala and Ser, or an analogue or mimetic thereof; or an anti-inflammatory drug; or a medicament for treating atherosclerosis; or a detectable label, and

R, F, L, C, W, S, D and P are the symbols indicating the amino acids Arg, Phe, Leu, Cys, Trp, Ser, Asp and Pro, respectively, according to IUPAC nomenclature and symbolism for amino acids.

It should be understood that the D- or L-amino acids of X₂ and/or X₃ may also take the form of analogues or mimetics of the corresponding amino acid.

In an embodiment the peptide comprises the amino acid sequence of SEQ ID NO: 1 (LSLERFLRCWSDAPA), wherein E represents the amino acid glutamic acid and wherein A represents the amino acid alanine. Thus, in this preferred embodiment X₂ in formula I is represented by the peptide LSLE (Leu-Ser-Leu-Glu), and X₃ and X₄ are both represented by the amino acid alanine.

In an alternative embodiment, the peptide comprises the amino acid sequence of SEQ ID NO: 2 (LSLERFLRCWSDSPR).

Yet in another embodiment the peptide comprises the amino acids that represent the consensus of SEQ ID NO: 1 and SEQ ID NO: 2, which consensus is represented by the amino acid sequence of SEQ ID NO: 3 (LSLERFLRCWSD) or truncated peptides thereof. A particularly preferred truncated peptide of SEQ ID NO: 3 is the peptide corresponding to the amino acid sequence of SEQ ID NO: 4 (LSLERFL). The minimal motif for binding to the synthetic SR-AI receptor is believed to be SEQ ID NO: 5 (LERFL), which is yet another preferred embodiment of a truncated peptide of SEQ ID NO: 3.

These peptides fore SRA targeting were synthesized on an automated peptide synthesizer (9050 Millipore, MA) using standard Fmoc solid-phase peptide synthesis. Crude peptides were purified on a preparative C8 RP-HPLC column (Altech, Deerfield, Ill.) using a JASCO PU-980 (Tokyo, Japan). The purity of the peptide, as checked by MALDI-TOF mass spectrometry and RP-HPLC, was at least 70%. Lyophilized peptides were stored at −20° C. under nitrogen until further use. Synthetic biotinylated bovine SR-AI was synthesized, purified and characterised as described by Suzuki et al. (Suzuki et al., 1999).

The invention thus also covers, for the diagnosis and/or treatment of inflammatory diseases, HR chelates associated:

1) with SR-targeting biovectors, in particular:

1a) biovectors described in documents U.S. Pat. No. 6,255,298, U.S. Pat. No. 6,458,845, WO 00/06147, WO 00/03704;

1b) modified lipoproteins, in particular acetylated LDLs (acLDL; Gurudutta et al., Nucl. Med. Biol. 2001 28: 235-24) and LDL oxides (oxLDL);

1c) AcLDL, OxLDL and LPS ligands described in Esbach et al., Hepatology, 199318: 537′; De Rike et al., J. Biol. Chem, 1994, 269: 824; Van Oosten et al., Infect. Immun. 1998, 66: 5107; Bijsterbosch et al., Nucleic Acids Res. 1997 25: 3290; Biessen et al, Mol. Pharmacol. 53: 262, 1998;

1d) peptides screened by phage display, capable of binding to the SR-AI receptor;

2) with folate receptor-targeting biovectors (these folate receptors are overexpressed in activated macrophages) for use in pathologies involving macrophage activation;

3) with peptides for targeting amyloid plaques leading in particular to Alzheimer's disease (for example, described in WO 01/74374, U.S. Pat. No. 6,329,531);

4) with biovectors of the CSF type (GCSF, GM-CSF, etc.) described, for example, in U.S. Pat. No. 6,491,893,

5) with antibodies or antibody fragments which target receptors overexpressed on macrophages (CD68, MRP6-18, etc.).

In the field of inflammatory diseases, the inventors have also obtained HR-BIOVECTORS in which the biovector is phosphatidylserine or a derivative of phosphatidylserine, for use in the diagnosis of macrophage-related diseases.

Phosphatidylserine (PS) is a membrane phospholipid located mainly in the inner face of the cell on the cytoplasmic side. Its overall negative charge stabilizes its polarity and prevents it from diffusing across the plasma membrane. PS serves as a recognition signal for the macrophage. The nature of this signal is still unknown (direct recognition, charge density, multiple receptors, inducible single receptor). According to the latest studies published, it is thought to be a direct interaction between PS and PS receptor (PSR) after induction of this receptor at the surface of certain macrophages present in the region undergoing a breakdown of homeostasis. In some macrophage, cells, PS also interacts with the scavenger receptors via their site of attachment for anionic phospholipids. PS is expressed on the inner face of the membrane, of all viable cells. In the event of cell suffering, or at the beginning of the apoptotic process, a membrane-bound translocase causes the PS to flip onto the extracellular face off the cell. This extracellular expression constitutes a recognition signal for macrophages, which recognize and phagocytose the suffering cell, thus avoiding a local inflammation.

The inventors have prepared a contrast agent bonded to PS or derivative, intended to be actively taken up by macrophages in order to image these various pathologies. Several chemical technical difficulties have been overcome in order to prepare the following PS biovectors, the chelate being coupled to the free NH₂ function:

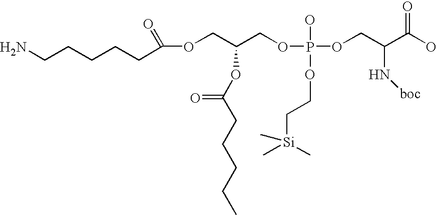

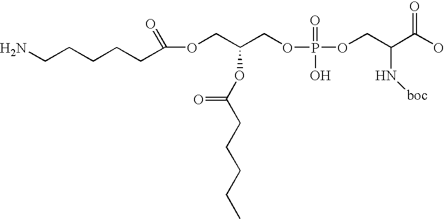

In order to completely control the structure of the final products, it has been necessary to prepare PS derivatives which are correctly functionalized (nature of the function involved in the coupling reaction with the HR chelate, position of said function on the PS molecule). The chemical functions selected are adapted for anchoring of the phospholipid to the paramagnetic probe in a selective and, effective manner. The presence of an amino acid residue on the polar portion of the PS is a source of further difficulties which had to be controlled by performing chemistry to protect/deprotect the free amine and acid functions, and/or by using chemical methods compatible with these functions. The groups which protect the amino acid residue are the conventional groups used in amino acid chemistry (Boc, tBu, Z, Bn, etc.). The functions that are preferred to provide the bond between the PS and the paramagnetic probe are NH₂, COOH and SH.

The inventors have also prepared vectorized products in which the phosphatidylserine lacks at least one of the fatty chains, the affinity not being altered in an interfering manner.

In addition to all the examples described above, the invention covers, in general, HR-BIOVECTOR compounds which are effective in diagnostic or therapeutic terms and which comprise a combination of at least one HR derivative and at least one biovector capable of targeting a ligand associated (directly or indirectly involved in and/or overexpressed in) a pathological process. The expression “effective in diagnostic terms” is intended to mean the fact that the HR compound has not lost, in an interfering manner, its selectivity relative to the corresponding non-HR compound, and that its relaxivity is sufficiently high to allow a significant improvement in diagnosis compared to known compounds, with the compound typically having a relaxivity r1 per Gd of at least 20, preferably of at least 30, 35 or 40 mMol⁻¹Gd⁻¹. By virtue of the present application, those skilled in the art have available to them the appropriate techniques for testing this diagnostic effectiveness.

Among the biovectors, in addition to those already mentioned above in the present application, mention will in particular be made of

1) The biovectors described in documents WO 01/97850 (targeting VEGF receptors and angiopoietin), U.S. Pat. No. 6,372,194 (polymer such as polyhystidine), WO 2001/9188 (fibrin-targeting polypeptide), WO 01/77145 (integrin-targeting peptide), WO 02/26776 (αvβ3 integrin-targeting peptide), WO 99/40947 (peptides targeting, for example, the KDR/Flk-I receptor, including R—X-K-X—H and R—X-K-X—H, or the Tie-1 and 2 receptors), WO 02/062810 and <<Müller et al, Eur. J. Org. Chem, 2002, 3966-3973 (glycosides of sialyl Lewis), WO 03/011115 (peptide with chelates coupled to the N and C terminal ends), Bioorganic & medicinal Chemistry letters 13, 2003, 1709-1712 (polyacrylamide targeting P selectine), Bioorganic & medicinal Chemistry letters 14, 2004, 747-749 (4-nitroimidazoles targeting tumors, WO 02/40060 (antioxidants such as ascorbic acid), U.S. Pat. No. 6,524,554 (targeting of tuftsin), WO 02/094873 (targeting of G-protein receptors GPCRs, in particular cholecystokinin), U.S. Pat. No. 6,489,333 (integrin antagonist and guanidine mimetic combination), U.S. Pat. No. 6,511,648 (quinolone targeting αvβ3 or αvβ5), US A 2002/0106325, WO 01/97861 (benzodiazepines and analogues targeting integrins), WO 01/98294 (imidazoles and analogues), WO 01/60416 (MMP inhibitors, in particular hydroxamates), WO 02/081497 (αvβ3-targeting peptides such as RGDWXE), WO 01/10450 (RGD peptides), U.S. Pat. No. 6,261,535 (antibodies or antibody fragments (FGF, TGFb, GV39, GV97, ELAM, VCAM, inducible with TNF or IL)), U.S. Pat. No. 5,707,605 (targeting molecule modified by interaction with its target), WO 02/28441 (amyloid-deposit targeting agents), WO 02/056670 (cathepsin-cleaved peptides), U.S. Pat. No. 6,410,695 (mitoxantrone or quinone), U.S. Pat. No. 6,391,280 (epithelial-cell-targeting polypeptides), U.S. Pat. No. 6,491,893 (GCSF), US 2002/0128553, WO 02/054088, WO 02/32292, WO 02/38546, WO 2003/6059, U.S. Pat. No. 6,534,038, WO 99/54317 (cysteine protease inhibitors), WO 0177102, EP 1 121 377, Pharmacological Reviews (52, no. 2, 179; growth factors PDGF, EGF, FGF, etc.), Topics in Current Chemistry (222, W. Krause, Springer), Bioorganic & Medicinal Chemistry (11, 2003, 1319-1341; αvβ3-targeting tetrahydrobenzazepinon derivatives).

2) Angiogenesis inhibitors, in particular those tested in clinical trials or already commercially available, especially:
antiogenesis inhibitors involving FGFR or VEGFR receptors, such as SU101, SU5416, SU6668, ZD4190, PTK787, ZK225846, azacycle compounds (WO 00/244156, WO 02/059110);
angiogenesis inhibitors involving MMPs, such as BB25-16 (marimastat), AG3340 (prinomastat), solimastat, BAY12-9566, BMS275291, metastat, heovastat;
angiogenesis inhibitors involving integrins, such as SM256, SG545, EC-ECM-blocking adhesion molecules (such as EMD 121-974, or vitaxin);
medicinal products with a more indirect mechanism of antiangiogenesis action, such as carboxiamidotriazole, TNP470, squalamine, ZD0101;
the inhibitors described in document WO 99/40947, monoclonal antibodies very selective for binding to the KDR receptor, somatostatin analogues (WO 94/00489), selectin-binding peptides (WO 94/05269), growth factors (VEGF, EGF, PDGF, TNF, MCSF, interleukins); VEGF-targeting biovectors described in Nuclear Medicine Communications, 1999, 20;
the inhibitory peptides of document WO 02/0665.12.

3) Biovectors capable of targeting receptors: CD36, EPAS-1, ARNT, NHE3, Tie-1, 1/KDR, Flt-1, Tek, neuropilin-1, endoglin, pleiotrophin, endosialin, Axl, alPi, a2ssl, a4 P1, a5pl, eph B4 (ephrin), laminin A receptor, neutrophilin 65 receptor, OB-RP leptin receptor, CXCR-4 chemokine receptor (and other receptors mentioned in document WO 99/40947), LHRH, bombesin/GRP, gastrin receptors, VIP, CCK, Tlr4.

4) Biovectors of the tyrosine kinase inhibitor type.

5) Known inhibitors of the GPIIb/IIIa inhibitor selected from: (1) the fab fragment of a monoclonal antibody for the GPIIb/IIIa receptor, Abciximab (ReoPro™), (2) small peptide and peptidomimetic molecules injected intravenously, such as eptifibatide (Integrilin™) and tirofiban (Aggrastat™).

6) Peptides which are fibrinogen receptor antagonists (EP 425-212), peptides which are IIb/IIIa receptor ligands, fibrinogen ligands, thrombin ligands, peptides capable of targeting atheroma plaque, platelets, fibrin, hirudin-based peptides, guanine-based derivatives which target the IIb/IIIa receptor.

7) Other biovectors or biologically active fragments of biovectors known to the person skilled in the art as medicinal products, with antithrombotic action, anti-platelet aggregation action, action against atherosclerosis, action against restenosis, and/or anticoagulant action.

8) Other biovectors or biologically active fragments of biovectors which target αvβ3, described in combination with non-HR DOTA in patent U.S. Pat. No. 6,537,520, selected from the following: mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustin, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, alpha-interferon, alpha2-interferon, beta-interferon, gamma-interferon, colony stimulating factor-1, colony stimulating factor-2, denileukin diftitox, interleukin-2, leutinizing hormone releasing factor.

9) Some biovectors which target specific types of cancer, for example peptides which target the ST receptor associated with colorectal cancer, or the tachykinin receptor.

10) Biovectors which use phosphine-type compounds.

11) Biovectors for targeting P-selectin, E-selectin (for example the 8-amino acid peptide described by Morikawa et al, 1996, 951).

12) Annexin V and any derivatives thereof or biovectors which target apoptotic processes.

13) Any peptide obtained by targeting technologies such as phage display, optionally modified with unnatural amino acids (http//chemlibrary.bri.nrc.ca), for example peptides derived from phage display libraries: RGD, NGR, CRRETAWAC, KGD, RGD-4C, XXXY*XXX, RPLPP, APPLPPR.

14) Other known peptide biovectors for targeting atheroma plaques, mentioned in particular in document WO 2003/014145.

15) Vitamins.

16) Ligands for hormone receptors, including hormones and steroids.

17) Opioid receptor-targeting biovectors.

18) TKI receptor-targeting biovectors.

19) LB4 and VnR antagonists.

20) Nitriimidazole and benzylguanidine compounds.

21) Biovectors recalled in Topics in Current Chemistry, vol. 222, 260-274, Fundamentals of Receptor-based Diagnostic Metallopharmaceuticals, in particular:
biovectors for targeting peptide receptors overexpressed in tumours (LHRH receptors, bombesin/GRP, VIP receptors, CGK receptors, tachykinin receptors, for example), in particular somatostatin analogues or bombesin analogues, optionally-glycosylated octreotide-derived peptides, VIP peptides, alpha-MSHs, CCK-B peptides;
peptides selected from: cyclic RGD peptides, fibrin-alpha chain, CSVTGR, tuftsin, fMLF, YIQSR (receptor: laminin).

22) Polysaccharides and ose derivatives, Glu-targeting derivatives.

23) Biovectors used for products of the smart type, for instance biovectors clivable in case of biochemical local reaction namely enzymatic.

24) Markers of myocardial viability (tetrofosmin and hexakis-2-methoxy-2-methylpropyl isonitrile).

25) Sugar and fat metabolism tracers.

26) Ligands for neurotransmitter receptors (D, 5HT, Ach, GABA, NA receptors).

27) Oligonucleotides.

28) tyrosine kinase inhibitors, for instance. Gefitinib, Erlotinib, Imatinib

29) antibodies known for their tumoral targeting.

Preferably, the biovectors used will have membrane targets, but use may also be made of biovectors having an intracellular target, for example activators of PPAR receptors (peroxisomal proliferator-activated receptors) known to reduce the risks of thrombosis due to plaque alteration, and some of which are, moreover, known to reduce MMP production. Proteases or extracellular components, present at the surface or in normal or pathological tissues may also be targeted.

Those skilled in the art, by virtue of the teaching of the present application (activity tests presented) and of the known screening techniques of the prior art (for example methods described in document WO 02/087632, CEREP 80 test), are able to screen the diagnostic or therapeutic effectiveness of these HR-BIOVECTOR compounds (in particular using biovectors described in the patents corresponding to the biovectors mentioned above).

The HR-biovector products of the present invention are therefore different from products which associate or which might associate:
a) firstly, chelates with low relaxivity (DTPA, DOTA, DO3A, BOPTA backbone, etc.) or with a relaxivity r1 and/or r2 of at least 20, 30, 35, 40, 50, 60, 70, 100, 150 mMol⁻¹Gd⁻¹ (polymers, 2nd- to 5th-generation dendrimers, chelates in a network, for example with cyclodextrines, etc.), in particular the non-HR derivatives mentioned at the beginning of the present application (and compounds derived from these non-HR derivatives in order to increase their relaxivity, for example by association, polymerization, crosslinking, grafting onto sugars, onto peptide or protein molecules, liposomes, micelles, polymers),
b) and, secondly, any biovector associated with pathological processes mentioned in the application.

According to another aspect, the invention relates to the MRI contrast products comprising an HR-BIOVECTOR compound as described above, in which the paramagnetic metal ion has the atomic number 21-29, 42-44, 58 or 70, preferably gadolinium.

According to another aspect, the invention relates to the X-ray-imaging or the CT-imaging contrast products comprising an HR-BIOVECTOR compound as described above, in which the heavy metal ion has the atomic number 21-31, 39-50, 56-80, 82, 83 or 90.

According to another aspect, the invention relates to radiopharmaceutical products comprising an HR-BIOVECTOR compound as described above, in which the HR chelate: is chelated with a radionucleide or a radiohalogen known to those skilled in the art, typically; gadolinium, technecium, chromium, gallium, indium, ytterbium, rhenium, lanthanium, yttrium, dysprosium, copper, or the like. Radiopharmaceutical compounds may also be prepared using a technique of the PET type with 18F (Vaidyanathan, G. and Zalutsky, M. R. Bioconjugate Chem. 1990, 1, 269-273; Vaidyanathan, G. and Zalutsky, M. R. Nucl. Med. Biol. 1992, 19, 275-281; Vaidyanathan, G. and Zalutsky, M. R. Bioconjugate Chem. 1994, 5, 352-364; Vaidyanathan, G. and Zalutsky, M. R. Nucl. Med. Biol. 1995, 22, 759-764; Sutcliffe-Goulden et al. Bioorg. Med. Chem. Lett. 2000, 10, 1501-1503).

According to another aspect, the invention relates to a method of radiopharmaceutical diagnosis and to a method of radiopharmaceutical treatment using a product as described above.

According to another aspect, the invention relates to the use of a product as described above, for preparing a diagnostic or radiopharmaceutical composition. The diagnostic and radiopharmaceutical compositions according to the invention can be used as described in applications US 2002/0090342, US 2002/0098149 and WO 02/055111 for anticancer indications.

For diagnosis by MRI, the intravenous administration by injection, usually in saline solution, is typically carried out at a dose of from 1 to 500 μmol Gd/kg.

For a radiopharmaceutical diagnosis, the intravenous administration by injection, usually in saline solution, is typically carried out at a dose of 1 to is 1100 mCi per 70 kg of body weight, preferably from 5 to 50 mCi.

For use as X-ray contrast agents, the concentration of heavy atom is typically from 0.1M to 5 M, with concentrations per intravenous administration of the order of 0.5 to 1.5 mmol/kg.

According to, another aspect, the invention also relates to the use of an HR chelate as described above, for preparing a composition intended for optical imaging.

The invention also relates to a method of imaging, comprising the synthesis of a compound comprising a paramagnetic metal according to the invention, capable of targeting a pathological region, its administration to a patient, and imaging by MRI. The invention also relates to a method of imaging, comprising the synthesis of a radiopharmaceutical compound according to the invention, capable of targeting a pathological region, its administration to a patient, and imaging by SPECT or planar gamma scintigraphy, or positron emission tomography.

Definitions

The term “salt” is defined, for example, in CRC Handbook of Chemistry and Physics, 65th Edition, CRC Press, Boca Raton, Fla., 1984. The term “pharmaceutically acceptable salt” refers to derivatives of the compounds according to the invention which are modified by forming acid or basic salts, for example inorganic or organic salts, acid salts of basic residues such as amines, alkaline salts of acid residues such as carboxylic acids (examples of salts: hydrochloric, hydrobromic, sulphuric, sulphamic, acetic, propionic, succinic, stearic, lactic, malic, tartaric, citric, glutamic), salts of meglumine or of lysine in particular.

A pharmaceutically acceptable dose refers to a dose that is suitable for therapeutical diagnostic use.

The term “alkyl” includes the saturated or unsaturated aliphatic hydrocarbon groups. The “C₁-Cₙ alkyls” include the C₁, C₂, C₃, C₄, C₅, C₆, C₇, C₈, . . . Cₙ alkyl groups, for example: methyl, ethyl, r-propyl, i-propyl, n-butyl, s-butyl, tert-butyl, n-pentyl. Examples of haloalkyl include trifluoromethyl, trichloromethyl and pentafluoroethyl.

The term “alkanoyl” includes in particular: formyl, alkyl as defined above substituted in the end position with a carbonyl, for example acetyl, propanoyl, butanoyl, pentanoyl, and the like.

The term “alkenyl” refers to linear or branched carbon chains with at least one carbon-carbon double bond.

The term “arylalkyl” refers

The term “alkynyl” refers to linear or branched carbon chains with at least one carbon-carbon triple bond.

The term “alkylamino” refers to N-substituted alkyls, including monoalkylamino (methylamino, ethylamino, propylamino, tert-butylamino, etc.) and dialkylamino (dimethylamino, diethylamino, methylpropylamino, etc.).

The term “halo” refers to elements of group 17, in particular fluoro, chloro, bromo, iodo.

The term “alkylenyl” refers to linear or branched carbon chains such as methylene, ethylene or 2-methylpropylene. The term “poloxyalkylene” refers to compounds such as polyoxyethylene or polyoxypropylene.

The term “natural amino acids” refers to the 20 amino acids involved in protein synthesis, such as glycine, alanine or methionine.

The alkoxys include in particular: methoxy, ethoxy, n-propoxy, i-propoxy, n-butoxy, s-butoxy, tert-butoxy, n-pentoxy, s-pentoxy. The cycloalkyls include in particular cyclopropyl, cyclobutyl, cyclopentyl. The “carbocycles” include monocycles, bicycles or tricycles, each cycle being partially unsaturated or aromatic, in particular: cyclopropyl, cyclobutyl, cyclopentyl, cyclohexyl, cycloheptyl, adamantyl, cyclooctyl, [3.3.0]bicyclooctane, [4.3.0]bicyclononane, [4.4.0]bicyclodecane, [2.2.2]bicyclooctane, fluorenyl, phenyl, naphthyl, indanyl, adamantyl. The alkaryls include in particular the aryl groups bearing an alkyl group containing 1, 2, 3, 4, 5, 6, 7, 8, 9 or 10 carbon atoms the aralkyls include the alkyl groups containing 1, 2, 3, 4, 5, 6, 7, 8, 9 or 10 carbon atoms, bearing an aryl group.

The heterocycloalkyls include in particular the alkyl groups containing 1, 2, 3, 4, 5, 6, 7, 8, 9 or 10 carbon atoms, bearing a heterocycle. The 5-, 6- or 7-membered stable monocyclic heterocylcles can be saturated, partially unsaturated or unsaturated, and comprise carbon atoms and 1, 2, 3 or 4 hetero atoms chosen from N, NH, O and S. These hetetocycles may be aromatic. The heterocycles include in particular those mentioned in U.S. Pat. No. 6,537,520, especially: pyridinyl, furanyl, thienyl, pyrrolyl, pyrazolyl, pyrrolidinyl, imidazolyl, indolyl, benzimidazolyl, 1H-indazolyl, oxazolidinyl, benzotriazolyl, benzisoxazolyl, oxindolyl, benzoxazolinyl and isatinoyl.

The invention covers, unless otherwise indicated, all the chiral, diastereoisomeric, racemic, in particular cis-trans, and L-D forms of the compounds described.

Examples of compounds obtained by the inventors are now described. Polymetallic compounds having the formula below will in particular be described:

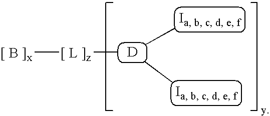

II is clear that monometallic compounds have also been obtained by the inventors using “bricks” similar or identical to those used for the polymetallic compounds.

Examples 1 to 10 describe the HR Ch signal component coupled with a linker L, where appropriate. The following are specified:
D′=D-H with D being a radical forming part of (E) and D′ being the intermediate of the same formula with the free amine function
the meaning of the branches AAG1AA28BR and AAG1 AA29 Br.

The branch AAG1AA28BR has the formula: 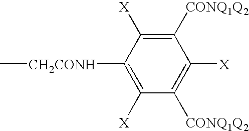
with Q₁=Q₂=CH₂(CHOH)₄CH₂OH and X═Br

R1 therefore represents:

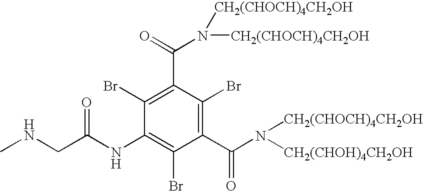

The branch termed AAG1AA29 Br has the formula: 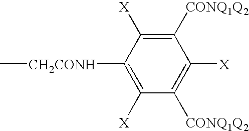
with Q₁=CH₂CHOHCH₂OH and Q₂=CH₂(CHOH)₄CH₂OH and x=Br

Examples 12 to 16 describe the biovector component: folate derivatives (Example 11), PS derivatives (Examples 12 and 13), peptides (Example 23).

The HPLC columns have the following characteristics:

Supersphere 60A RP-SELECT B® 4 μm, (125×4.6 mm) (Merck®)

Symmetry® C18 100 Å; 5 μm; (250×4.6 mm) (Waters®)

Symmetry® C18 5μ, 100 Å (100×4.6 mm) (Waters®)

HyperCarb® 5 μm 250 Å; (250×4.6 mm) (hypersil®)

X-TERRA MS® C18 5μ, (250×4.6 mm) (Waters®)

Licrospher® RP18 100 Å, 5 μm, (250×4.6)(Merck®).

SEC (Steric Exclusion Chromatography):

Carried out on a succession of 4 columns (d=8 mm, I=30 cm) sold by Shodex® (JP) under the references OH Pack SB-HQ, containing polyhydroxymethacrylate gel, the exclusion limits of which, determined with Pullulan®, are successively: 10⁶ KD (SB-804); 10⁵ KD (SB-803); 10⁴ KD (SB-802.5); 10⁴ KD (SB 802.5); eluent: 70/30 v/v aqueous solution of NaCl (0.16 M)/CH₃CN, flow rate 0.8 ml/min. T=30° C.:

### EXAMPLE 1

Compounds of formula V where x=2, and —S₁-T′-S₂— is, with S₁═S₂═(CH₂)₂,

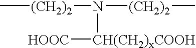

while Z₁ is

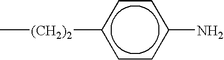

and Z₂ is —(—CH₂)₂—COOH

a)

40.4 g of methyl 2-bromo-4-(4-nitrophenyl)butyrate in solution in 50 ml of CH₃CN are added dropwise to a suspension of 20 g of 1,4,7,10-tetraazacyclododecane in 140 ml of CH₃CN. After stirring for 24 h at 25° C., the solution is filtered, and washed with CH₃CN and then with 200 ml of diethyl ether. After filtration, the product in hydrobromide form is recrystallized from 200 ml of CH₃CN. m=42 g; Mp=170° C.

HPLC:

Lichrospher C18® column

water-KH₂PO₄ 0.01M/CH₃CN

tr: 2.5 min
b) Reaction with Y′″Br=

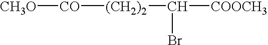

A suspension containing 20 g of the compound obtained in stage a) and 20 g of Na₂CO₃ in 400 ml of CH₃CN is brought to reflux temperature for 15 min, before adding, dropwise, 40 g of methyl 2-bromoglutarate. After stirring at reflux for 24 h and then overnight at 25° C., the medium is filtered and the solvent is then evaporated off and the residue is dissolved in 100 ml of CH₂Cl₂. The organic phase is washed with water and then dried over sodium sulphate, before elimination of the solvent by evaporation under reduced pressure. The residue is dissolved in the minimum volume of 1M aqueous HCl solution. This solution is washed with the same volume of diethyl ether and then brought to pH 4 with NaHCO₃ before being extracted with diethyl ether. After evaporation of the organic phase, the residue is purified by chromatography on silica (Merck® Si 60) elution being carried out with a heptane/CH₃COOC₂H₅ mixture (40/60 v/v then 30/70 v/v); m=8 g.

HPLC:

Symmetry® C18 column; water-TFA pH 3/CH₃CN; tr: 22-29 min

c) Hydrolysis of the Methyl Ester Groups

10 g of the compound obtained according to stage b) are dissolved in 20 ml of a 12N aqueous HCl solution and the mixture is brought to reflux for 24 h. After cooling, the solution is evaporated off and the residue is dissolved in water. After concentrating under vacuum, 7.7 g of crude product are obtained.

d) Complexation of Gadolinium with the Above Compound

The solution of 5 g of the crude product above in 30 ml of H₂O is brought to pH 5.2 by adding 5M NaOH before adding 1.2 g of Gd₂O₃. The medium is heated at 80° C. for 2 h 30 min, during which the pH is maintained between 5.2 and 5.5 by adding a 6M aqueous HCl solution. After cooling to 25° C., the medium is run into 250 ml of C₂H₅OH at 10° C. The precipitate obtained after washing with C₂H₅OH is dried; m 5 g.

HPLC:

Symmetry® C18 column; water-TFA pH 2.8/CH₃CN; tr: 31-34 min

e) Reduction of the Nitro Group

5 g of the gadolinium complex in the form of a sodium salt are dissolved in 70 ml of water and hydrogenated under pressure (palladium-on-charcoal at 10%, 25° C. under a hydrogen pressure of 3×10⁵ Pa for 6 h.) 5 g of product are obtained in the form of a sodium salt.

HPLC: Symmetry® C18 column; water-TFA pH 2.8/CH₃CN; tr: 17-21 min

### EXAMPLE 2

Compound of formula: II′₂ with x=2
-GNH— is 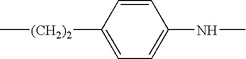
R is 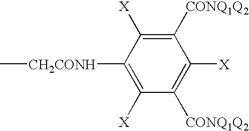
with Q₁=Q₂=CH₂(CHOH)₄CH₂OH and X═Br
D′ is 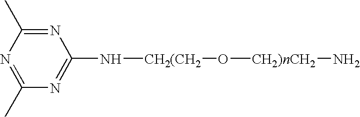
with n=2.
   a) Condensation with the Triazine Ring

A solution of 0.66 g of 2,4,6-trichloro-1,3,5-triazine in 9 ml of dioxane is added, with stirring, to a solution of 7.6 g of the compound of stage e) Example 1 in 75 ml of water in the presence of NaHCO₃, of pH=7.7. After stirring for 6 h at ambient temperature, the reaction medium is stored overnight at 4° C.

Mass spectrum: Mode ES⁺ m/z=950 with z=2

HPLC: Symmetry® C18 column; water-TFA pH 3/CH₃CN; tr: 41 min

b) Coupling of the Amine R—NH₂

30.06 g of amine R—NH₂, pH=6.65, are added, at ambient temperature and with vigorous stirring, to the solution of stage a). 0.2 ml of 6N HCl is added so as to obtain a pH=6.2. 0.47 g of NHS and then 5.8 g of EDCI are then added to the reaction medium. After stirring at ambient temperature for 3 h, one volume of water is added to the reaction medium, then the mixture is ultrafiltered through a polyethersulphone membrane (Pall®) with a cut-off threshold of 1 KD, and the retentate is evaporated to a volume of 100 ml and then run into 1000 ml of EtOH under cold conditions. The precipitate formed is isolated. Mass obtained=29 g.

Mass spectrum: Mode ES⁻ m/z=2141.6 with z=4

HPLC: Symmetry® C18 column; water-TFA pH 2.8/CH₃CN; tr: 22 min

c) Introduction of the Diamine

45 ml of 2-2′-(ethylenedioxy)bisethylamine are diluted in 173 ml of DMSO at 65° C. The intermediate previously prepared is added. After stirring for 1 h at 65° C. and for 1 h at ambient temperature, the reaction medium is run into 1730 ml of ethanol. The precipitate obtained is filtered off and washed with ethanol. The product obtained is redissolved in 750 ml of water so as to be purified by ultrafiltration through a membrane with a cut-off threshold of 1 KD.

Mass spectrum: Mode ES− m/z=1735.2 with z=5

HPLC: Licrospher® RP18 column; water/CH₃CN; tr: 21 min

### EXAMPLE 3

Compound of formula: 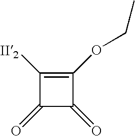
with: x, -GNH—, R, Q₁, Q₂, X, D′ and n as defined in Example 2 and D-H=D′.

1.38 g (1.59×10⁻⁴ mol) of the compound of stage c) of Example 2 are dissolved in 3.5 ml of dimethyl sulphoxide (DMSO) at 70° C. The reaction medium is brought back to ambient temperature. 0.135 g of 3,4-diethoxy-3-cyclobutene-1,2-dione are mixed with 0.4 ml of ethanol and the solution obtained is introduced into the reaction medium in a single step 34 μl of triethylamine are added and the reaction medium is stirred at ambient temperature for 5 hours. The mixture is precipitated from 200 ml of ethanol and the stirring is maintained overnight at ambient temperature. The precipitate is filtered off and dried under vaccum. 1.29 g of product are isolated.

HPLC: Superspher RP-SELECT B column; water-TFA pH 3/CH₃CN; Tr: 7.60 min

Mass spectrum: Mode ES⁻ m/z=2199.8 with z=4

### EXAMPLE 4

Compound of formula VII₁ in which x=2

a)

A solution of 102 g of the ester methyl 2-bromo-4-nitrophenylbutyrate in 100 ml of CH₃CN is added to a suspension of 70 g of 3,6,9,15-tetraazabicyclo[9.3.1.]pentadeca-1(15),11,13-triene in 800 ml of CH₃CN in the presence of 910 ml of anion exchange resin in the form of a strong base (Amberlite® IRA458). After stirring at 25° C. for 3 days, filtration of the resin and evaporation, the oil obtained is purified by chromatography on a column of 5 kg of silica (Merck®, 40-60 μm), elution being carried out with a CH₂Cl₂/CH₃OH mixture (70/30 v/v). 38 g of product are obtained.

HPLC: Symmetry® C18 column; water-TFA pH 3/CH₃CN; tr: 15 min

¹³C NMR (125 MHz, d6-DMSO, 30° C.):

δ (ppm): 160.1 (C₁); 53.8 (C_{2,4}); 45-45.4-45.7 (C_{5,7,8}); 51.3 (C₁₀); 161.6 (C₁₁); 119.3 (C₁₂); 119.6 (C₁₄); 137.6 (C₁₃); 51.7 (O—CH₃); 172.8 (C═O); 65.8 (C—N); 31.06-31.45 (CH₂—CH₂); 149.6-129.6-122 (Ar); 145.6 (Ar—NO₂).
b) Reaction with Y′″Br=

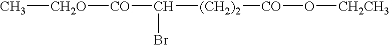

6.8 g of K₂CO₃ and 13 g of ethyl 2-bromoglutarate are added to a solution of 7 g of the compound obtained in stage a) in 70 ml of CH₃CN and 35 ml of diisopropyl ether, and the mixture is then left to stir for 24 h at reflux. After elimination of the salts by filtration, and concentration of the solution, the oil obtained is purified by chromatography on silica (Merck® 40-63 μm), elution being carried out with a CH₂Cl₂/acetone mixture (70/30 v/v). 6 g of solid product are obtained.

HPLC: Symmetry® C18 column; water-TFA pH 2.8/CH₃CN; tr: 33 min

c) Hydrolysis of the Ethyl Ester Groups

6 g of the compound obtained in stage b) are added to a 10 ml solution of 12N HCl, and the mixture is then stirred at its reflux temperature for 48 h. After filtration and concentration, the residue is purified by chromatography on silianized silica gel (Merck® 0.063-0.20 μm), elution being carried out with an H₂O/CH₃OH mixture to give 2.8 g of product.

HPLC: Symmetry® C18 column; water-TFA pH 2.8 CH₃CN; tr: 17-19 min

d) Gadolinium Chelate of the Above Compound

2 g of GdCl₃.6H₂O are introduced into 35 ml of a solution, at pH 5, of 3.9 g of the compound obtained in stage c), and the mixture is maintained at 50° C. for 5 h, during which time the pH is adjusted if necessary by adding an aqueous (2N) NaOH solution. The medium is subsequently filtered and evaporated; 4 g of slightly acidic cation exchange resin Chelex® 100 (Bio-Rad) are added to the oil obtained, dissolved in 40 ml of water. After stirring at 25° C. for 2 h, the resin is removed by filtration and the solution is evaporated off to give 4.5 g of product.

HPLC: Symmetry® C18 column; water-TFA pH 2.8/CH₃CN; tr: 15.6-18.7 min

e) Reduction of the Nitro Group

By applying the same procedure as for stage e) of Exampe 1, 4 g of product are obtained from 4.5 g of the compound obtained in stage d).

HPLC: Symmetry® C18 column; water-TFA pH 2.8/CH₃CN; tr: 8.6-9.5 min

### EXAMPLE 5

Compound of formula: II″ₐ₂ with x=2
-GNH— is 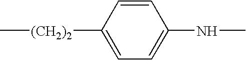
R is 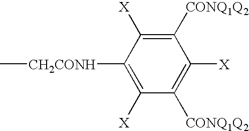
with Q₁=Q₂CH₂(CHOH)₄CH₂OH and X═Br
D′ is 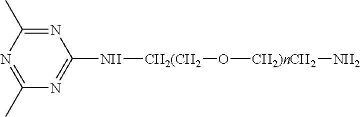
with n=2
   a) Condensation with the Triazine Ring

5 g of the compound obtained in stage e) of Example 4 are condensed with 2,4,6-trichloro-1,3,5-triazine according to the protocol described in stage a) of Example 2. After reaction for 3 h, the pH is brought back to 7 with a sodium hydrogen carbonate solution. The solution obtained is conserved overnight in the refrigerator.

Mass spectrum: Mode ES⁻ m/z=852.7 with z=2;

HPLC: Symmetry® C18 column; water-TFA pH 2.95/CH₃CN; tr=20-25 min.

b) Coupling of the Amine R—NH₂

14 g of the amine of formula RNH₂, 2.68 g of 1-(3-dimethylaminopropyl)-3-ethylcarbodiimide hydrochloride (EDCI) and 0.326 g of the sodium salt of (N-hydroxysuccinimidyl)-3-sulphonic acid (NHS) are added to a water-dioxane solution containing 4.2 g of the compound obtained in stage a). The coupling is carried out according to the protocol described in stage b) of Example 2, so as to obtain 18 g of product.

Mass spectrum: Mode ES⁻ m/z=1024.9 with z=6

HPLC Symmetry® C18 column; water-TFA pH 2.7 CH₃CN; tr=13 min.

c) Introduction of the Diamine

20 ml of 2-[2-(2-aminoethoxy)ethoxy]ethylamine are introduced into a solution of 70 ml of dimethyl sulphoxide containing 18 g of the compound obtained in stage b). The mixture is stirred at 50° C. for 1 hour. After cooling to 25° C., the solution is run into 1000 ml of ethanol, the precipitate formed is dissolved in 400 ml of water and the solution is ultrafiltered through a membrane with a cut-off threshold of 1 KD. After evaporation of the retentate, the product obtained is purified by preparative HPLC. 1.5 g of solid are thus obtained.

Mass spectrum Mode ES⁻ m/z=2087.2 with z=3

HPLC: Symmetry® C18 column; water-TFA pH 2.7/CH₃CN; tr=11 min.

### EXAMPLE 6

Compound of formula:

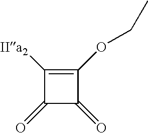

With: x, -GNH—, R, Q₁, Q₂, X, D′ and n as defined in Example 5 and D-H=D′

0.5 g of the compound obtained in Example 5c is dissolved in, 2 ml of DMSO at 80° C., the reaction medium is placed at ambient temperature and then 17 μl of triethylamine and 59 μl of 3,4-diethoxy-3-cyclobutene-1,2-dione are added and the reaction medium is stirred at ambient temperture for 5 h. The reaction medium is precipitated from 20 ml of ethanol. The precipitate is filtered off, washed with ethanol and then dried under vacuum. 500 mg of product are obtained.

Mass spectrum:

Mode ES⁻ m/z=6390.5 with z=1

HPLC: Superphere RP Select B® (column; water-TFA pH 2.8/CH₃CN; tr=15.9 min.

### EXAMPLE 7

Compound of formula VI with x=2 according to the method of Table 2

a) Compound of Formula VI(1) with B=ethyl

22 g of 13-bromo-3,6,9,15-tetraazabicyclo[9.3.1.]pentadeca-1(15),11,13-triene are introduced in 440 ml of CH₃CN in the presence of 48 g of calcinated K₂CO₃ and the mixture is maintained at 80° C. for 1 h before adding a solution of 93 g of ethyl 2-bromoglutarate in 100 ml of CH₃CN; the reaction medium is then stirred at 80° C. for 20 h, then cooled to ambient temperature and filtered and the solvent is evaporated off. The residue is taken up with 500 ml of an aqueous 1N HCl solution in the presence of one volume of diethyl ether. After separation of the organic phase, the aqueous phase is neutralized with NaHCO₃ and then extracted with CH₂Cl₂. After washing with water and then drying over magnesium sulphate, the organic phase is concentrated and the residue is purified on a column of silica (Merck® 500 g, d=10 cm), elution being carried out with CH₃COOC₂H₅.

m=37;

HPLC: Symmetry® C18 column; water-TFA pH 3/CH₃CN; tr=26 min.

b) Compound of Formula VI (2)

23.5 g of 3-(tert-butyloxycarbonylamino)propene, 25.3 ml of triethylamine, then 3.4 g of triphenylphosphine and, finally, 1.8 g of palladium acetate are added to a solution of 28 g of the compound obtained in stage a), dissolved in 400 ml of toluene. After heating overnight at 80° C. under an inert atmosphere, the medium is evaporated off and the residue is taken up with an aqueous hydrochloric acid solution (pH=1). The aqueous phase is washed with 1 volume of diethyl ether and then of toluene, before being brought to pH 6 by adding NaOH (1N). After extraction of the aqueous solution with CH₂Cl₂, the organic phase, which has been dried over magnesium sulphate, is evaporated. A brown oil is obtained. m=17 g,

HPLC: Symmetry® C18 column; water-TFA pH 3/CH₃CN; tr=14-19 min.

c) Compound of Formula VI (3)

3 g of catalyst palladium-on-charcoal at 10% are added to 17 g of the compound obtained in stage b), dissolved in 350 ml of CH₃OH, and the reaction medium is then stirred at 20° C. for 2 h 30 under 4×10⁵ Pa of hydrogen. After filtration through Clarcel®, the solvent is evaporated off and 16.8 g of crude oil are obtained.

HPLC: Symmetry® C18 column; water-TFA pH 3/CH₃CN; tr=15-16-21 min.

d) Hydrolysis of the Ethyl Ester Groups

20 g of the compound obtained in stage c), dissolved in 50 ml of a 5N aqueous NaOH solution and 80 ml of CH₃OH, are heated at 70° C. for 18 h. After concentrating the reaction medium, the residue is taken up in water and the solution, brought to pH 5.5-6 with a few drops of acetic acid, is concentrated before being purified by chromatography on a column (d=15 cm) containing 1 kg of silanized silica (Merck® 0.063-0.200 μm), elution being carred out with water. After concentrating to dryness, 9.3 g of white crystals are obtained,

HPLC: Symmetry® C18 column; H₂SO₄ in water (0.037 N)/CH₃CN; tr=16.7-17.5-17.9 min

e) Gadolinium Complexation

8.7 g of the compound obtained in stage d) are dissolved in 70 ml of water, then 2.1 g of Gd₂O₃ are added in a single step and the entire mixture is heated at 60° C. for 3 h 45 min, maintaining the pH between 5.5 and 6 by adding a 1N aqueous NaOH solution. After filtration, the reaction medium is evaporated off and the residue is crystallized from ethanol. 9.6 g of white crystals are obtained.

HPLC: Symmetry® C18 column; H₂SO₄ in water (0.037 N)/CH₃CN; tr=31-31.7-32.2-33 min

f) Freeing of the Amine

A solution of 9 g of the complex obtained in stage e) in 180 ml of CF₃COOH is kept stirring at 25° C. for 3 h before eliminating the liquid under reduced pressure. The residue is taken up in diethyl ether and the suspension is filtered. After elimination of the solvent, the residue is introduced portionwise into a suspension of at least 5 ml of weak anionic resin (OH⁻) in 50 ml of water; at the end of the addition, the pH, which is stable, should be 8 to 8.5. The resin is then separated by filtration, the solvent is eliminated and the residue is precipitated by adding ethyl ether.

### EXAMPLE 8

Preparation of the Intermediate Chelate of Formula VIII 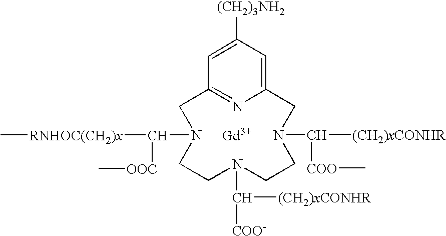
and R represents 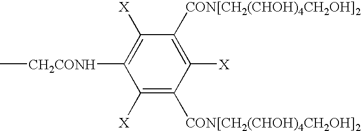
X═Br
a) Coupling of the Amine R—NH₂

6 g of compound obtained in stage e) of Example 7 and 26.5 g of the amine RNH₂ are dissolved in 200 ml of water and 7.6 g of EDCI and 0.4 g of NHS are added. The mixture is kept stirring at around pH 6 for 24 hours, with addition of a 1N aqueous NaOH or HCl solution if necessary. After evaporation of the solvent, the residue is crystallized by adding ethanol. The 35 g of yellow crystals obtained are dissolved in 200 ml of water and the solution is ultrafiltered with a polyethersulphone membrane (Pall®) having a cut-off threshold of 1 kD.

The retentate is concentrated and purified by chromatography on a column of silanized silica (Merck®) (diameter: 7 cm, height: 33 cm), elution being carried out with water and then water/methanol mixtures (90/10 V/V to 80/20). The fractions containing the desired product are concentrated until elimination of the solvents. The residue, dissolved in 50 ml of water, is treated with 20 ml of anionic resin in OH⁻ form (HP 661 from Rohm and Haas) and then treated with carbon black at 45° C. After filtration and elimination of the solvents, 10 g of white crystals are isolated.

HPLC: Symmetry® C18 column; H₂O/CH₃CN; tr=15 min

SEC: conditions No. 1 tr=40 min

b) Deprotection of the Amine

The solid obtained above is dissolved in 200 ml of trifluoroacetic acid. After stirring at ambient temperature for 3 hours the liquid is eliminated under reduced pressure and the residue is crystallized by adding diethyl ether. 8.8 g of white crystals, trifluoroacetate of the amine of formula VIII are thus obtained.

HPLC: Symmetry® C18 column; H₂O/CH₃CN; tr=4-5.3-5.9 min

### EXAMPLE 9

Compound of formula: II′″₂ with x=2
-GNH— is
   (—CH₂)₃—NH
R is 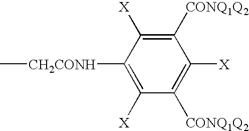
with Q₁=Q₂=CH₂(CHOH)₄CH₂OH and X═Br
D′ is. 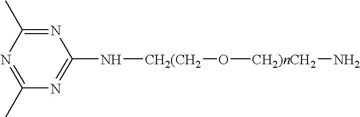
with n=2.
   a) Condensation of the Triazine Ring

0.132 g of potassium carbonate is added to a solution containing 4 g of the compound obtained in stage b) of Example 8 in 25 ml of distilled water. A solution of 0.080 g of 2,4,6-trichloro-1,3,5-triazine in 5.5 ml of dioxane is added and the pH is then brought to 8.4 by adding K₂CO₃. After neutralization with cationic resin in H⁺ form, the solvents are evaporated off and the residue is taken up in absolute ethanol. The precipitate is isolated.

3.7 g of product are isolated.

Mass spectrum: Mode ES⁺ m/z=2099 with z=4

HPLC: Symmetry® C18 column; water/CH₃CN; tr=10.8 min.

b) Introduction of the Diamine

Starting with a solution containing 3.7 g of the compound obtained in stage a), in 30 ml of dimethyl sulphoxide, with 0.220 g of potassium carbonate and with 1.3 g of diamine 2-[2-(2-aminoethoxy)ethoxy]ethylamine according to the protocol described in stage 5c), and after purification by preparative HPLC, 1 g of product is obtained.

Mass spectrum:

Mode ES⁻ m/z=1700.3 with z=5

HPLC: Lichrosphere C18® column; water-TFA pH 3.3/CH₃CN; tr=13.8 min.

### EXAMPLE 10

Compound of formula: 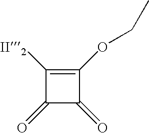
with: x, -GNH—, R, Q₁, Q₂, X, D′ and n as defined in Example 9 and D-H=D′

0.8 g of the compound obtained in stage b) of Example 9 and 73 μl of 3,4-diethoxy-3-cyclobutene-1,2-dione are dissolved in 3 ml of dimethyl sulphoxide. After the addition of 20 μl of triethylamine, the medium is left at ambient temperature for 4 h. The product obtained by precipitation in 20 ml of ethanol is filter-dried and then washed twice with 10 ml of ethanol and of ether. 650 mg of white crystals are obtained.

HPLC: Superpher RP Select B® (column; water-TFA pH 2.8/CH₃CN; tr=12 min.

### EXAMPLE 11

J. Am. Chem. Soc, 1997, 119, 10004-10013
a) Compound of Formula:

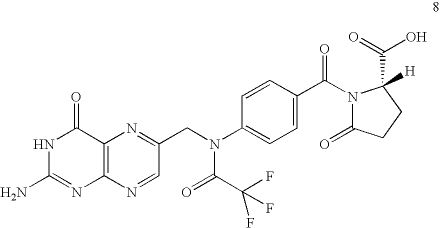

50 g (0.113 mol) of folic acid are suspended in 500 ml of THF in a 1 litre three-necked flask equipped with a refrigerant, a magnetic stirrer, a thermometer and a dropping funnel, and then the entire mixture is cooled to 0° C. Trifluoroacetic acid anhydride (128 ml, 0.906 mol, 8 eq) is added dropwise, making sure that the temperature does not exceed 5° C. The mixture is stirred at 5° C. for 6 h. The three-necked flask is wrapped in aluminium foil and left overnight in the refrigerator. Evaporation is carried out at 40° C. in a rotary evaporator until an oil is obtained. Precipitation from 3 litres of ether and stirring at ambient temperature for 2 h are carried out. Filtration over sintered glass and drying under vacuum at 30° C. overnight are carried out. Mass obtained 54.4 g

HPLC: Symmetry® C18 column; water-TFA pH 3/CH₃CN; tr=33 min.
b) Compound of Formula:

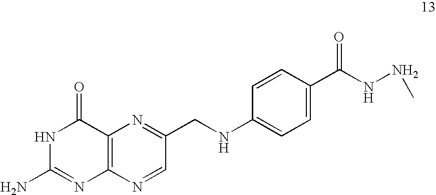

54.4 g (0.088 mol) of the product obtained above are added, in small fractions, to 540 cc (11 mol, 125 eq) of hydrazine hydrate, in a 1 litre round-bottomed flask equipped with a magnetic stirrer. The reaction medium is stirred at ambient temperature for 24 h. Precipitation from 4 litres of methanol and stirring at ambient temperature for 4 h are carried out. Filtration over sintered glass is carried out, and the precipitate is cleared with methanol and then with ether. The precipitate is dried overnight at 40° C. in a ventilated oven. Mass obtained: 30 g

HPLC: Symmetry® C18 column; water-TFA pH 3/CH₃CN; tr-10.50 min.
c) Compound of Formula:

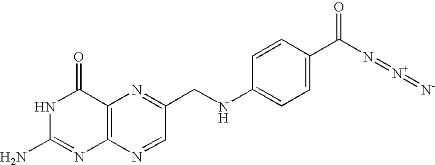

10 g (0.0306 mol) of product obtained above and 0.15 g (1.53.10⁻³ mol, 0.05 eq) of KSCN are introduced into a 250 cc three-necked flask equipped with a refrigerant, a magnetic stirrer, a thermometer and a dropping funnel. The reaction medium is cooled to −10° C. without stirring. 134 ml of trifluoroacetic acid are introduced and the mixture is stirred at −10° C. until complete dissolution. n-Butylnitrite (3.15 g, 0.0306 mol, 1 eq) is added dropwise, making sure that the temperature does not exceed −5° C. The mixture is stirred at −10° C. for 6 h. The mixture is allowed to return to ambiant temperature and 1 g of NaN₃ is added (0.015 mol, 0.5 eq). The mixture is stirred at ambient temperature overnight. The reaction medium is introduced into a dropping funnel and run dropwise into 350 ml of isopropanol pre-cooled to 0° C. The mixture is stirred for 2 h, making sure that the temperature does not exceed 10° C. The precipitate is filtered off over sintered glass. The precipitate is washed with 400 ml of CH₃CN and stirred overnight at ambient temperature. Filtration is carried out over sintered glass. The precipitate is washed with 200 ml of water for 1 h at ambient temperature. Filtration over sintered glass and clearing with ether are carried out. Drying is carried out under vacuum at ambient temperature overnight. Mass obtained: 13.5 g

HPLC: Symmetry® C18 column; water-TFA pH 3/CH₃CN; tr=35.40 min.
d) Compound of Formula:

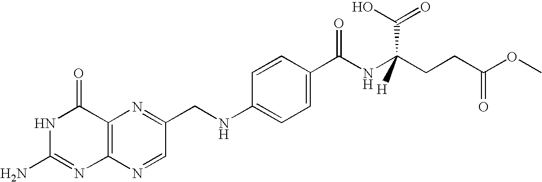

5.18 g (0.0153 mol) of compound prepared in the preceding step and 2.72 g (0.0168 mol, 1.1 eq) of L-glutamic acid 5 methyl ester are suspended in 60 ml of DMSO, in a 100 ml three-necked flask equipped with a magnetic stirrer and a dropping funnel. The reaction medium is cooled to 5° C. and 1,1,3,3-tetramethylguanidine (3.84 ml, 0.0306 mol, 2 eq) is introduced slowly, making sure that the temperature does not exceed 15° C. The mixture is stirred at 10° C. for approximately 15 minutes until a homogeneous medium is obtained, and then stirred at ambient temperature for 4 h. A slight amount of insoluble material is filtered off. Precipitation from 800 ml of acetone and stirring overnight at ambient temperature are carried out. The precipitate is filtered off over sintered glass and cleared with ether. It is dried under vacuum at ambient temperature. Mass obtained: 6.4 g

HPLC: Symmetry® C18 column; water-TFA pH 3/CH₃CN; tr=22.60 min.
e) Compound of Formula:

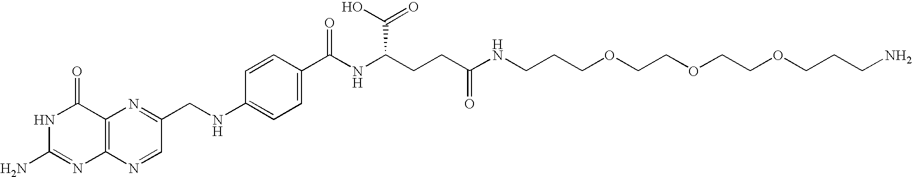

9.74 g (0.0214 mol) of product prepared above are dissolved in 60 ml of DMSO at ambient temperature, in a 500 ml single-necked flask equipped with a magnetic stirrer. 235 ml (1.07 mol, 50 eq) of 4,7,10-trioxa-1,13-tridecanediamine are added in a single step and the mixture is stirred at ambient temperature for 48 h. A slight amount of insoluble material is filtered off. The filtrate is precipitated from a mixture consisting of 1500 ml of CH₃CN and 1500 ml of ether. Stirring is carried out at ambient temperature for 3 h. The precipitate is filtered off over sintered glass. Drying is carried out under vacuum at ambient temperature. Mass obtained: 9.5 g

HPLC: Symmetry® C18 column; water-TFA pH 3/CH₃CN; tr=16 min.

### EXAMPLE 12

a) Condensation of the 2 Fatty Chains

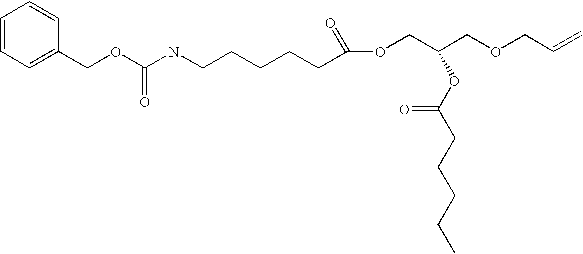

A solution of 0.61 g of DMAP (dimethylaminopyridine) and 41.5 g of DCC (dicyclohexylcarbodiimide) in 50 ml of CH₂CL₂ is added, dropwise, at 0° C., to a solution of 28 g of 3-allyloxy-1,2(R)-propanediol and 12.5 g of N-CBZ-6-aminohexanoic acid in 500 ml of CH₂Cl₂. After stirring at 0° C. for 1 h and then at ambient temperature for 1 h, the mixture is filtered over sintered glass and then concentrated and redissolved in 500 ml of CH₂Cl₂ in the presence of 25 ml of hexanoic acid. A solution of 41 g of DCC and 1.22 g of DMAP in 50 ml of CH₂Cl₂ is added, dropwise and at ambient temperature, to this mixture. The medium is stirred at ambient temperature for 1 h and is then again filtered and evaporated. 25 g of yellow oil are obtained.

HPLC: Symmetry® C18 (4.6×100) column; water-TFA pH 3.2/CH₃CN; tr=16.5 min.
b) Deprotection of the Allyl

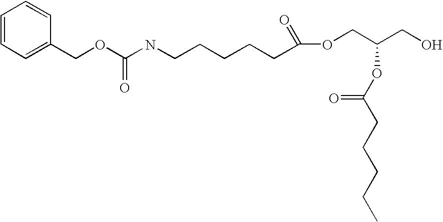

0.5 g of Pd(OAC)₂ and 2.33 g of triphenylphosphine are added to a solution of 25 g of compound obtained in stage a) dissolved in 300 ml of acetic acid. After heating at 80° C. for 5 days, the medium is filtered through glass fibre and then evaporated The residue is purified by preparative HPLC.

HPLC: Symmetry® C18 (4.6×100) column; water-TFA pH 3.2/CH₃CN; tr=14, 14.5, 17.7 min.

NMR:

¹H NMR (CDCl₃): 7.45 (5 aromatic H), 5.25 (O—CH—Ar), 4.65-4.20 and 3.90 (2CH₂O— and CHO from glycerol), 3.37 (OCO—NH—CH₂—), 2.53 (m, 2 OCO—CH—)

¹³C NMR (CDCl₃): 173.9 (2OCO—CH₂), 156.9 (OCO—NH), 137 (aromatic C^{IV}), 128.9/128.5 (5 aromatic CH), 68.5 (CH from glycerol), 67.0/65.4 (2 CH₂ from glycerol), 41.2 (OCO—NH-CH₂—)
c) Phosphorylation

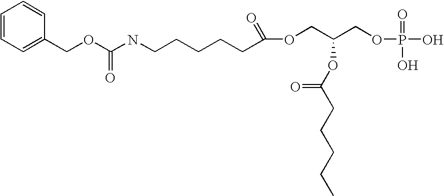

A solution of 6 g of the compound obtained in stage b), dissolved in 46 ml of toluene, is added, dropwise and at 0° C., to a soltution of 1.92 ml of POCl₃ and 2.85 ml of triethylamine dissolved in 18 ml of heptane. After stirring at 0° C. for 1 h and then at ambient temperature overnight, the medium is hydrolyzed with 30 ml of water, at ambient temperature for 2 h, and then: separated by settling out. The organic phase is concentrated and then purified by acid/base washing. A yellow oil is obtained, m=5.4 g.

HPLC: X-TERRA® MS C18 column; (NH4)₂CO₃—H₂O pH 9/CH₃CN; tr=13.3-14 min.

NMR:

³¹P NMR (CDCl₃): −3.5 ppm
d) Coupling of Serine

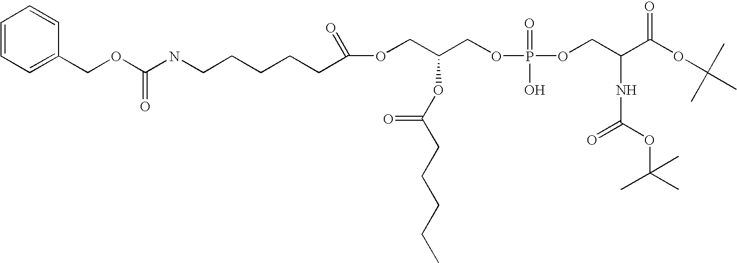

A solution made up of 9.3 g of triisopropylbenzenesulphonyl chloride dissolved in 60 ml of pyridine is added, dropwise and at ambient temperature, to a solution of 5.3 g of compound obtained in stage c) and 5.35 g of N-(tert-butoxycarbonyl)-L-serine t-butyl ester (Boc-L-Ser-OtBu) in 80 ml of pyridine. This mixture is stirred at ambient temperature for 18 h and then hydrolyzed with 120 ml of water. After concentrating, the residue is purified by preparative HPLC.

HPLC: Lichrospher C18® column; water-TFA pH 3.3/CH₃CN; r=11.7 min.

NMR:

¹H NMR (CDCl₃): 4.50-4.10 (CH₂O—P from glycerol, CH₂O—P from serine CH—NH from serine), 1.40 (6CH₃ from tBu)

¹³C NMR (CDCl₃): 82.9/80.5 (2C(CH₃)₃), 64.9 (CH₂O—P from glycerol), 64.3 (CH₂O—P from serine), 56.7 (CH—NH from serine), 28.7/28.4 (6HH₃ from tBu)

³¹P NMR (CDCl₃): −2.5 ppm
e) Deprotection of the CBZ

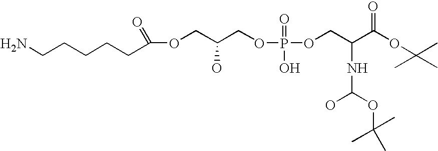

0.5 g of the catalyst palladium-on-charcoal at 10% is added to 3.8 g of compound obtained in stage d), dissolved in 200 ml of 2-methyl-2-propanol, and the reaction medium is then stirred at 30° C. for 6 h under 20×10⁵ Pa of hydrogen. After filtration through glass fibre, the solvent is evaporated off and the residue is purified by preparative HPLC.

HPLC: Symmetry® C18 (4.6×100) column; water-TFA pH 3.2/CH₃CN; tr=7.6 min.

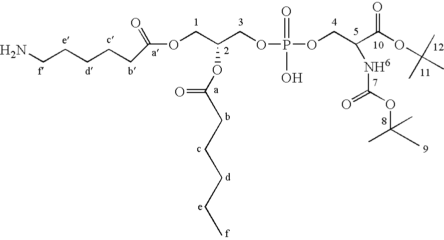

¹H NMR (CDCl₃):

6.26-6.10 (1 ddd, H₆, ³J_{H5-H}₆=7.9 Hz), 4.60/3.99 (2 m, H₁), 4.54 (1 m, H₂), 4.26/4.16 (2 m, H₃), 4.25 (1 m, H₅), 4.15/4.11 (2-m, H₄), 2.91 (1 m, H_{f}), 2.41 (1 m, H_{b′}), 2.30 (1 m, H_{b}), 1.60 (1 m, H_{c}), 1.72 (1 m, H_{c′}), 1.69 (1 m, H_{e′}), 1.47 (1 m, H_{d′}), 1.44 (1 m, H_{g}), 1.41 (1 m, H₁₂), 1.29 (1 m, Hₑ), 1.28 (1 m, H_{d}), 0.86 (1 t, H_{f})

¹³C NMR (CDCl₃):

173.6 (C_{a′}), 173.3 (Cₐ), 1.69.5 (C₁₀), 155.8 (C₇), 82.1 (C₁₁), 79.5 (C₈), 71.5 (C₂), 65.8 (C₄), 63.9 (C₁), 63.2 (C₃), 55.2 (C₅), 39.5 (C_{f}), 33.8 (C_{b}), 33.7 (C_{b′}), 28.4 (C₁₂), 28.0 (C₉), 26.7 (C_{e′}), 25.3 (C_{d′}), 24.4 (C_{c}), 24.0 (C_{c′}), 22.1 (Cₑ), 13.6 (C_{f})

³¹P NMR (CDCl₃):

0.85/0.75 ppm.

### EXAMPLE 13

a) Phosphorylation

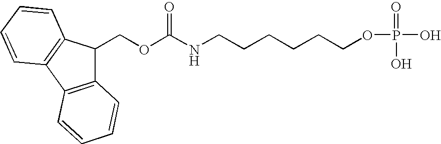

1.23 ml of POCl₃ and 1.63 ml of triethylamine are dissolved in 9 ml of heptane at 0° C. The alcohol (N(Fmoc)-6-aminohexanol, 3 g) is dissolved in CH₂Cl₂ and added dropwise to the above solution without exceeding 0° C. The reaction medium is then stirred at 0° C. for 1 h and then at ambient temperature for 18 h. 30 ml of water are added and the medium is stirred at ambient temperature for 2 h. The two phases are separated; the organic phase is concentrated. The resulting solid is washed with water, filtered and dried under vacuum. 3 g of product are obtained.

HPLC: X-TERRA® MS C18 column; (NH4)₂CO₃—H₂O pH 9/CH₃CN; tr=14.1 min.
b) Coupling of Serine

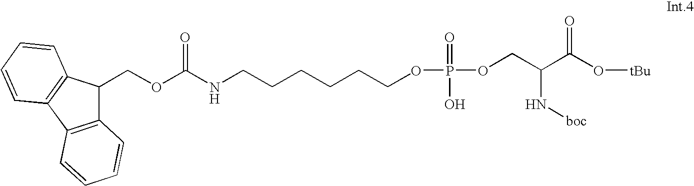

3 g of the compound obtained above and 3.73 g of Boc-Ser-OtBu are dissolved at ambient temperature in 54 ml of pyridine. A solution of 6.49 g of TIS (triisopropylbenzene sulphochloride) in 42 ml of pyridine is added dropwise and the medium is stirred at ambient temperature for 48 h. The reaction medium is then hydrolyzed with 30 ml of water, stirred at ambient temperature for 4 h and then concentrated to dryness. The crude obtained is taken up in 100 ml of Et₂O, and the precipitate formed is filtered off over sintered glass. The filtrate is concentrated and then purified by chromatography on silica, elution being carried out with 95% CH₂Cl₂-5% MeOH. 3.9 g of product are obtained.

HPLC: X-TERRA® MS C18 column; (NH4)₂CO₃—H₂O pH 9/CH₃CN; tr=20 min.
c) Cleavage of Fmoc

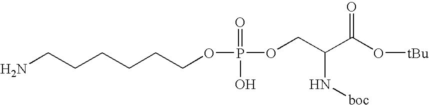

3 g of compound obtained in in the preceding stage and 0.62 ml of piperidine in acetonitrile are stirred at ambient temperature for 12 h. A white precipitate forms. The reaction medium is filtered and the crystals are washed with 2 times 50 ml of CH₃CN and then recrystallized from 100 ml of CH₃CN. 0.9 g of product is obtained.

HPLC: Symmetry® C18 column; water/MeOH; tr=13.6 min.

### EXAMPLE 14

### a) Benzyl 5-{[(2S-2-{[(9H-fluoren-9-ylmethoxy)carbonyl]amino}-3-(1H-indol-3-yl)propanoyl]amino}pentylcarbamate

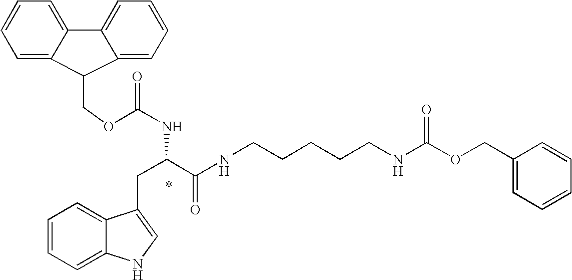

15.63 g of ((2R)-2-{[(9H-fluoren-9-ylmethoxy)carbonyl]amino}-3-(1H-indol-3-yl)propanoic acid) are solubilized at ambient temperature in 300 ml of tetrahydrofuran. 10 g of benzyl 5-aminopentylcarbamate and then 5.1 ml of triethylamine are: added. The entire mixture is stirred at ambient temperature for 5 minutes. 5.94 g of 1-hydroxybenzotriazole hydrate and then 8.43 g of 1-(3-dimethylaminopropyl)-3-ethylcarbodiimide hydrochloride are subsequently added to the reaction medium and the entire mixture is stirred at ambient temperature for 24 hours. The insoluble material is removed by filtration. The filtrate is concentrated under vacuum. The oil obtained is run into 150 ml of water and the entire mixture is vigorously stirred at ambient temperature for 1 hour. The precipitate obtained is washed with vigorous stirring in 100 ml of water, filtered, and then washed with 200 ml of ethyl ether. The precipitate is filtered off and dried. 22.78 g are isolated.

TLC: SiO₂ Merck®; CH₂Cl₂/MeOH-80/20; rf=0.94 min.

HPLC: Symmetry® C18 column; water-TFA pH 2.95/CH₃CN; tr-40 min.

Mass spectrum: Mode ES⁺ m/z=645 with z=1

### b) Benzyl 5-{[(2S)-amino-3-(1H-indol-3-yl)propanoyl]amino}pentylcarbamate

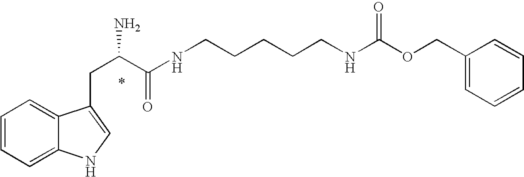

20 g of the compound obtained above are solubilized at ambient temperature in 280 ml of tetrahydrofuran. 41.4 ml of piperidine and 20 ml of water are then added. The entire mixture is stirred at ambient temperature for 3 hours. The reaction medium is concentrated under vacuum. The oil obtained is purified on silica. Elution is carried out with [CH₂Cl₂/CH₃OH] (9.5/0.5). After evaporation and drying under vacuum, 12.77 g of product are isolated.

TLC: SiO₂ Merck®; CH₂Cl₂/MeOH-95/5; rf=0.64 min.

HPLC: Symmetry® C18 column; water-TFA pH 2.95/CH₃CN; tr=19.5 min.

Mass spectrum: Mode ES+ m/z=423 with z=1

### c) tert-Butyl (12S)-12-(1H-indol-3-ylmethyl)-(15R)-15-isobutyl-3,11,14-trioxo-1-phenyl-2-oxa-4,10,13-triazaheptadecan-17-oate

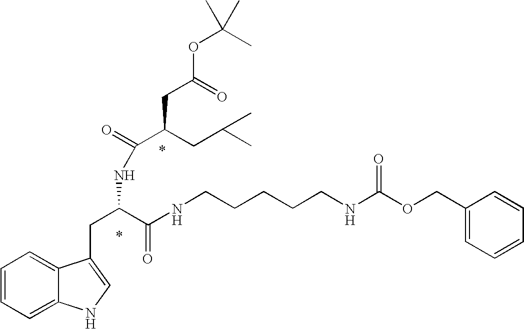

9.16 g of (2(R)-(2-tert-butoxy-2-oxoethyl)-4-methylpentanoic acid) synthetised according to the procedure described in (J. Med. Chem. 1998, Vol 41, No. 2 p 209), are solubilised at ambient temperature in 160 ml of tetrahydrofuran. A homogenous solution made up of 16.81 g of the compound prepared in the preceding stage in 165 ml of tetrahydrofuran is added in a single step, followed, successively, by 8.3 ml of triethylamine, 6.45 g of hydroxy-1-benzotriazole hydrate and 9.15 g of 1-(3-dimethylaminopropyl)-3-ethylcarbodiimide hydrochloride. The entire mixture is stirred at ambient temperature for 12 hours. The insoluble material is eliminated by filtration. The filtrate is concentrated under vacuum. The oil obtained is purified on silica. The correct product is eluted with [CH₂Cl₂/CH₃OH] (98/2). After evaporation and drying under vacuum, 24 g of product are isolated.

TLC: SiO₂ Merck®; CH₂Cl₂/MeOH-95/5; rf=0.42 min.

HPLC: Symmetry® C18 column; water-TFA pH 2.95/CH₃CN; tr=25.7 min.

Mass spectrum: Mode ES+ m/z=635 with z=1

### d) (12S)-12-(1H-Indol-3-ylmethyl)-15-isobutyl-3,11,14-trioxo-1-phenyl-2-oxa-4,10,13-triazaheptadecan-17-oic acid

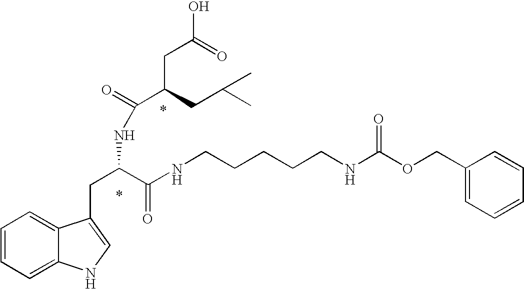

1.4 g of dithioerythritol are added to a mixture made up of 100 ml of CH₂Cl₂ and 100 ml of trifluoroacetic acid. The mixture is stirred, under argon, until complete dissolution is obtained. 10 g of the compound prepared above are then added. The entire mixture is stirred at ambient temperature for 30 minutes and then concentrated under vacuum. The oil obtained is purified on silica [CH₂Cl₂/CH₃OH] (98/2). After evaporation and taking up with ether, the product is filtered and dried. 6.19 g are isolated.

TLC: SiO₂ Merck®; CH₂Cl₂/MeOH-80/20; rf=0.57 min.

HPLC: Symmetry® C18 column; water-TFA pH 2.90/CH₃CN; tr=20.8 min.

Mass spectrum:

Mode ES⁺ m/z=579 with z=1

### e) Benzyl (8S)-8-(1H-indol-3-ylmethyl)-(11R)-11-isobutyl-7,10,13-trioxo-16-phenyl-15-oxa-6,9,14-triazahexadec-1-ylcarbamate

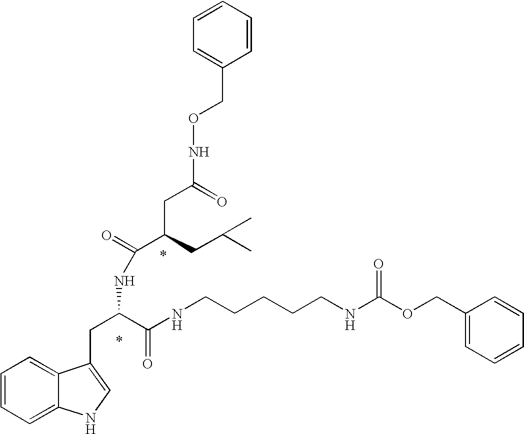

6.11 g of the product obtained above are dissolved at ambient temperature in 160 ml of tetrahydrofuran. The reaction medium is cooled to 0° C. 1.69 g of O-benzylhydroxylamine hydrochloride, 3 ml of triethylamine, 1.86 goof 1-hydroxybenzotriazole hydrate and then 2.63 g of 1-(3-dimethylaminopropyl)-3-ethylcarbodiimide hydrochloride are successively added to the reaction medium and the entire mixture is stirred at ambient temperature for 24 hours. The insoluble material is removed by filtration. The filtrate is concentrated under vacuum. The oil obtained is run into 200 ml of water and the entire mixture is vigorously stirred at ambient temperature for 1 hour. The precipitate obtained is filtered off, finely ground in a mortar, and then washed again with vigorous stirring in 100 ml of water for 30 minutes at ambient temperature. The precipitate is filtered off and dried under vacuum. 5.7 g of product are isolated.

TLC: SiO₂ Merck®; CH₂Cl₂/MeOH-50/50; rf=0.29 min.

HPLC Symmetry C18 column; water-TFA pH 2.80/CH₃CN; tr=22.8 min.

Mass spectrum:

Mode ES⁺ m/z=684 with z=1

### f) N¹-[(1S)-2-[(5-Aminopentyl)amino]-1-(1H-indol-3-ylmethyl)-2-oxoethyl]-N⁴-hydroxy-(2R)-2-isobutylsuccinamide

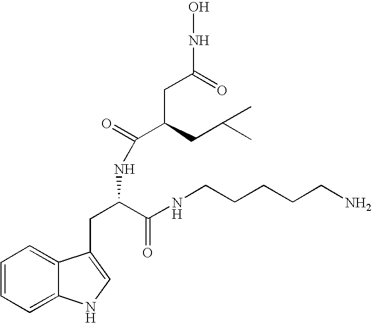

0.5 g of the product prepared above is dissolved in a solution made up of 75 ml of ethanol and 50 μl of concentrated HCl, in a 100 ml reactor equipped with a magnetic stirrer. 1 g of 50% hydrated Pd/C is added to the solution. The entire mixture is vigorously stirred for two hours at ambient temperature under 1 atmosphere of hydrogen. The catalyst is removed by filtration through clarcel. The solution obtained is filtered (0.45μ) and the filtrate is concentrated under vacuum. 0.32 g of product is isolated.

HPLC: Symmetry® C18 column; water-TFA pH 2.80/CH₃CN; tr=9.90 min.

Mass spectrum: Mode ES+ m/z=460 with z=1

### EXAMPLE 15

a) Protection of the Diamine

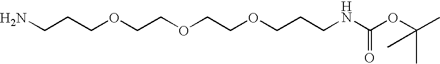

0.113 mol of 4,7,10-trioxa-1,13-tridecanediamine is dissolved in 200 ml of CH₂Cl₂. A solution of 0.038 mol of dibutyl dicarbonate (Boc₂O) in 50 ml of CH₂Cl₂ is added by means of a dropping funnel. The reaction medium is stirred at ambient temperature for 18 h. After having concentrated the reaction medium to 150 ml, the organic phase is washed with 2 times 150 ml of water. The chloromethylenic phase is dried over Na₂SO₄; filtered, washed, and then concentrated. The oil obtained is purified on silica (AcOEt/MeOH). 5.7 g of product are obtained.

HPLC: Symmetry® C18 column; water-TFA pH 2.80/CH₃CN; tr=9.5 min.

Mass spectrum: Mode ES− m/z=320.9 with z=1
b) Coupling of the Protected Amino Acid

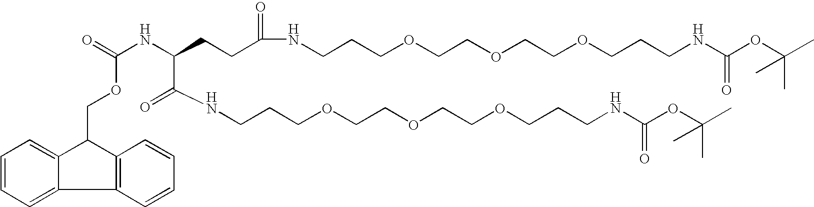

5.4 mmol of N-α-Fmoc-L-glutamic acid (Fmoc-Glu-OH) are suspended in 50 ml of CH₂Cl₂ under argon. 2 eq of N-hydroxysuccinimide (NHS) are added thereto, followed by 2 eq of dicyclohexylcarbodiimide (DCC). The reaction medium is stirred for 45 min and then filtered. The precipitate is washed with CH₂Cl₂. A solution of 11 mmol of compound obtained above (stage 15a) in 50 ml of CH₂Cl₂ is added by means of a dropping funnel. The reaction medium is stirred at ambiant temperature for 2 h. 30 ml of water are added. The mixture is allowed to separate by settling out, and the organic phase is recovered, dried over Na₂SO₄, filtered, washed, and then concentrated to 2/3. The solution obtained is purified by chromatography on SiO₂(CH₂Cl₂/MeOH). After evaporation of the fractions, an oil is obtained. m=3.8 g

TLC: SiO₂ Merck®; AcOEt/MeOH-90/10; rf=0.4 min.

HPLC Symmetry® C18 column; water-TFA pH 2.80/CH₃CN; tr=41 min.

Mass spectrum: Mode ES− m/z=974.6 with z=1
c) Deprotection of the Fmoc

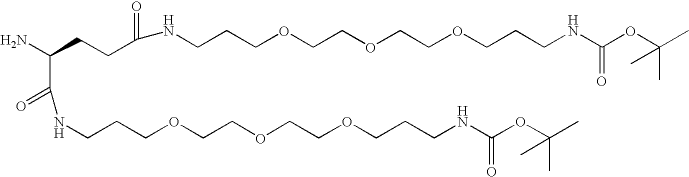

1.02 mmol of the compound obtained in the preceding stage is solubilized in 6 ml of CH₃CN. 10 ml of CH₃CN containing 20% of piperidine are added. The reaction medium is stirred at ambient temperature under argon for 3 h. After evaporation of the solvent, the residue obtained is purified by chromatography on SiO₂(CH₂Cl₂/MeOH). After evaporation of the solvents, 0.66 g of oil is obtained.

TLC: SiO₂ Merck®; CH₂Cl₂/MeOH-80/20; rf=0.35.

Mass spectrum:

Mode ES+ m/z=751.5 with z=1
d) Coupling of the Pteroic Acid

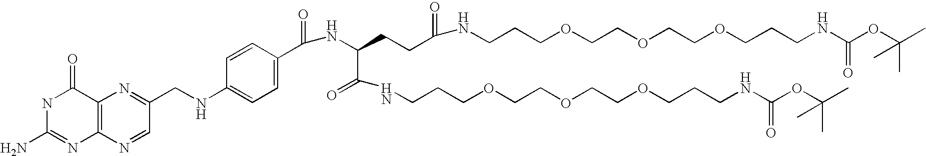

0.8 mmol of pteroic acid is suspended in 25 ml of dimethyl sulphoxide (DMSO) in the presence of 0.8 mmol of the compound prepared above. 75 mg of hydroxybenzotriazole (HOBT) and 200 mg of dicyclohexylcarbodiimide (DCC) are added at ambient temperature with thorough stirring. The reaction medium is stirred for 72 h in the dark at 40° C. The reaction medium is run into 250 ml of Et₂O. A gum is obtained, which is filtered and then resuspended in 10 ml of water. Filtration and ashing with water are carried out, followed by drying in a desiccator under vacuum in the presence of P₂O₅. 320 mg of amber crystals are obtained.

HPLC: Symmetry® C18 column; water-TFA pH 2.80/CH₃CN; tr=34 min.

Mass spectrum:

Mode ES+⁻ m/z=1046.5 with z=1
e) Deprotection:

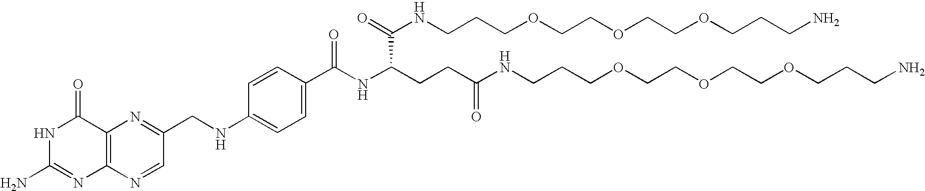

0.28 mmol of compound prepared above is dissolved in 5 ml of trifluoroacetic acid (TFA). After magnetic stirring at ambient temperature for 15 min, the reaction medium is run into 25 ml of Et₂O. The precipitate obtained is filtered off, washed with Et₂O, and then dried in a desiccator under vacuum in the presence of P₂O₅. The orange-coloured crystals obtained are purified by preparative HPLC. 110 mg of product are obtained.

HPLC: Symmetry® C18 column; water-TFA pH 2.80/CH₃CN; t=11.3 min.

Mass spectrum: Mode ES+⁻ m/z=846.5 with z=1

### EXAMPLE 16

### Parallel Synthesis

a) Coupling

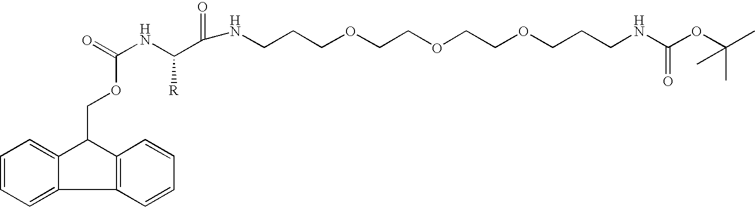

Formula

| | | | Analytical HPLC* | |
|---|---|---|---|---|
| | | | Tr | MS |
| R = | Name | Example | (mn) | m/z |
| 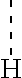 | Gly | 16-1 | 3.4 min | 600.4 |
| 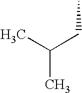 | Leu | 16-2 | 3.8 min | 656.4 |
| 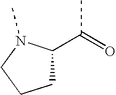 | Pro | 16-3 | 3.5 min | 640.4 |
| 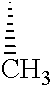 | Ala | 16-4 | 3.5 min | 614.4 |
| 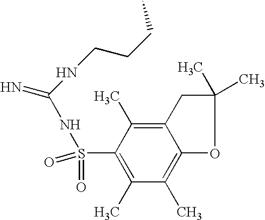 | Arg | 16-5 | 4 min | 951.5 |
| 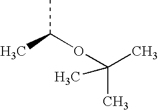 | Thr | 16-6 | 4.1 min | 700.5 |
| 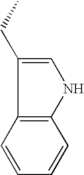 | Trp | 16-7 | 3.8 min | 729.4 |

Procedure for the Synthesis of Example 16-4

253 mg of NHS, followed by 454 mg of DCC, are added to 685 mg of Fmoc-Ala-OH in 20 ml of dichloromethane (DCM). The mixture is left to stir at ambient temperature for 30 min. 700 mg of compound obtained in Example 15 a), diluted in 10 ml of dichloromethane, are added. The mixture is left to stir, at ambient temperature for 1 h. Filtration and washing with 2×10 ml of water are carried out. After evaporation of the DCM, the product obtained is purified by chromatography on silica (DCM/methanol (95/5)).

b) Deprotection of the Fmoc

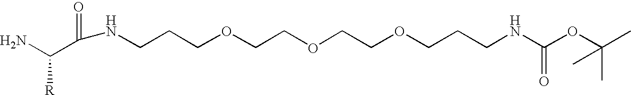

Formula

| | | | Analytical HPLC* | |
|---|---|---|---|---|
| | | | Tr | MS |
| R = | Name | Example | (mn) | m/z |
| 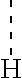 | Gly | 16-1 | 1.68 min | 378.3 |
| 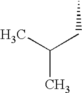 | Leu | 16-2 | 2.02 min | 434.4 |
| 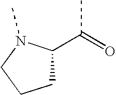 | Pro | 16-3 | 1.73 min | 418.3 |
| 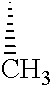 | Ala | 16-4 | 1.7 min | 392.51 |
| 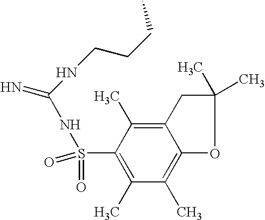 | Arg | 16-5 | 2.52 min | 729.5 |
| 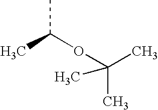 | Thr | 16-6 | 2.10 min | 478.4 |
| 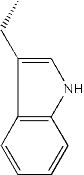 | Trp | 16-7 | 2.08 min | 507.4 |

Procedure for the synthesis of Example 16-4

500 mg of compound obtained in stage a) are introduced into 5 ml of acetonitrile (ACN). A solution of piperidine at 20% is then added and the reaction medium is left to stir for 3 h at ambient temperature. The product obtained is filtered, washed with ACN and purified by chromatography on silica.

c) Coupling of Pteroic Acid

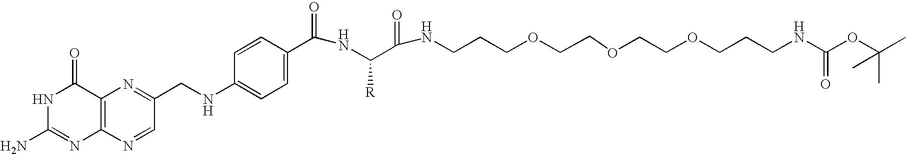

Formula

| | | | Analytical HPLC* | |
|---|---|---|---|---|
| | | | Tr | MS |
| R = | Name | Example | (mn) | m/z |
| 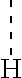 | Gly | 16-1 | 2.15 min | 672.4 |
| 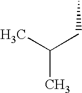 | Leu | 16-2 | 2.57 min | 728.5 |
| 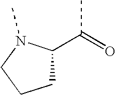 | Pro | 16-3 | 2.28 min | 712.5 |
| 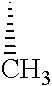 | Ala | 16-4 | 2.20 min | 686.5 |
| 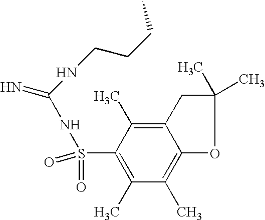 | Arg | 16-5 | 2.95 min | 1023.6 |
| 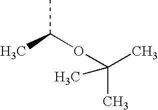 | Thr | 16-6 | 2.73 min | 772.6 |
| 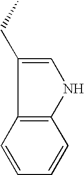 | Trp | 16-7 | 2.55 min | 801.5 |

Procedure for the Synthesis of Example 16-4

132 mg of pteroic acid, followed by 57 mg of HOBT and 121.5 mg of 1-(3-dimethylaminopropyl)-3-ethylcarbodiimide hydrochloride (EDCI), are added to 165.5 mg of compound obtained in the preceding stage, dissolved in 5 ml of DMSO. The reaction mixture is left overnight at 40° C. with stirring. Filtration, washing with 2×50 ml of diethyl ether and 2×20 ml of water and centrifugation are carried out, and the yellow precipitate formed is recovered.

d) Deprotection of the tBoc

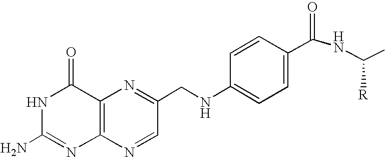

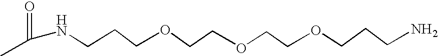

| | | | Analytical | |
|---|---|---|---|---|
| | | | HPLC* | LC/MS |
| R = | Name | Example | Tr (mn) | m/z |
| 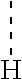 | Gly | 16-1 | 1.2 min | 572.3 |
| 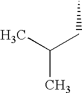 | Leu | 16-2 | 1.7 min | 628.3 |
| 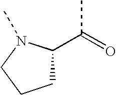 | Pro | 16-3 | | |
| 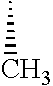 | Ala | 16-4 | 1.4 min | 586.3 |
| 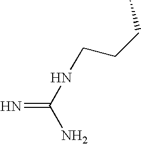 | Arg | 16-5 | 0.5 min | 671.4 |
| 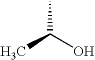 | Thr | 16-6 | 0.9 min | 616.3 |
| 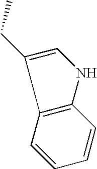 | Trp | 16-7 | 1.7 min | 701.4 |

*HPLC = column = Symmetry ®C18, 100 Å, 3.5 μm, L = 5 cm, d = 2.1 mm

*HPLC=column=Symmetry® C18, 100 Å, 3.5 μm, L=5 cm, d=2.1 mm

Eluent: CF₃COOH in water (pH=2.8)/CH₃CN

Procedure for the Synthesis of Example 16-4

The compound obtained above is deprotected in TFA. After 30 min, the product is precipitated from ethyl ether and washed 3 times with ethyl ether.

### EXAMPLE 17

### Parallel Synthesis

Compound of formula: E

Such that B═

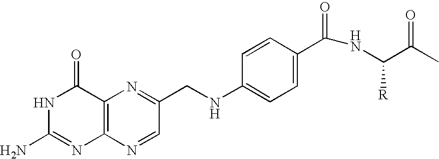

L=

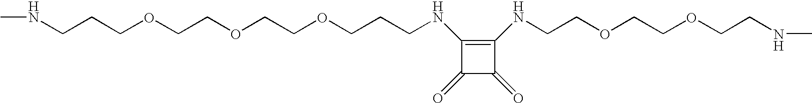

HR Ch is such that r is 2 and Iₐ has the formula II′1 such that:
x=2
-GNH— is 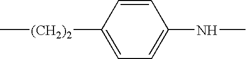
R is 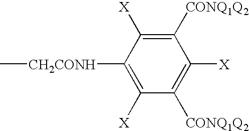
with Q=Q₂=CH₂(CHOH)₄CH₂OH and X═Br
D is 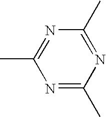
with q=1

and x, y, z=1.

| | | | Analytical | |
|---|---|---|---|---|
| | | | HPLC** | MS |
| R = | Name | Example | Tr (mn) | m/z |
| 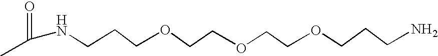 | Gly | 16-1 | | |
| 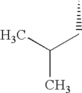 | Leu | 16-2 | 16 | 9384 ± 1 |
| 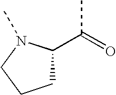 | Pro | 16-3 | | |
| 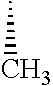 | Ala | 16-4 | 13.8 | 9343 ± 0.8 |
| 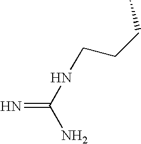 | Arg | 16-5 | | |
| 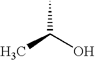 | Thr | 16-6 | 13.6 | 9375 ± 1.7 |
| 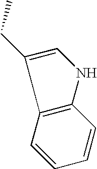 | Trp | 16-7 | 18 | 9460 ± 1.9 |

**HPLC = column = RP select B ®, 60 Å, 5 μm, L = 12.5 cm, d = 4.6 mm,

** HPLC=column ═RP select B®, 60 Å, 5 μm, L=12.5 cm, d=4.6 mm,

Procedure for the Synthesis of Example 16-4

1 g of compound obtained in Example 3 is dissolved in 12 ml of water, and the pH is adjusted to 9.2 with a sodium carbonate solution. The compound obtained in stage 16 d), dissolved beforehand in water, is added. The reaction mixture is left at ambient temperature for 48 h with stirring. At the end of the reaction, the medium is neutralized and the product is purified by preparative chromatography.

### EXAMPLE 18

Compound of formula: E

such that B═

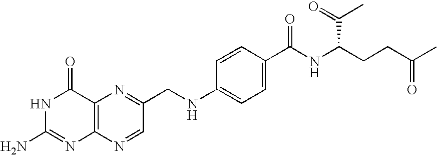

L=as defined in Example 17

HR Ch=as defined in Example 17

with x=1 and y, z=2

673 mg of compound prepared in Example 3 are dissolved in 7 ml of water, and the pH is then adjusted to 9 with an Na₂CO₃ solution 40 mg of compound obtained in Example 15 e) in solution in 1 ml of acetonitrile are added. The reaction medium is stirred at ambient temperature for 24 h and the pH is then adjusted to 6.5 with a 1N aqueous HCl solution. The reaction medium is evaporated to dryness under reduced pressure. The product obtained is purified by preparative HPLC.

HPLC: Supersphere RP Select B® column; water-TFA, pH 2.80/CH₃CN; tr=12.8 min.

Mass spectrum: Mode ES⁻ m/z=18361 with z=1

### EXAMPLE 19

Compound of formula: E

such that B═

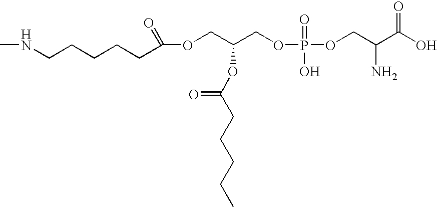

L=

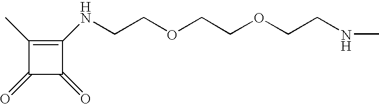

HR Ch= as defined in Example 17

with x, y, z=1

a) Condensation

3.3 g of the intermediate obtained in Example 3 are dissolved in 33 ml of water and the pH is adjusted to 9 with an Na₂CO₃ solution. 271 mg of the intermediate obtained in Example 12 e) in solution in 3.3 ml of acetonitrile are added. The reaction medium is stirred at ambient temperature for 24 h and the pH is adjusted to 5.7 with a 1N aqueous HCl solution. The reaction medium is evaporated to dryness under reduced pressure. The product obtained is purified by preparative HPLC. 2 g of product are obtained.

HPLC: Supersphere RP Select B® column; water-TFA, pH 2.80/CH₃CN; tr=22.8 min.

Mass spectrum: Mode ES⁻ m/z=9384.8 with z=1

b) Deprotection

1.7 g of the compound obtained in the preceding stage are dissolved in 30 ml of TFA. The mixture obtained is stirred at 40° C. for 3 hours. This solution is precipitated from 300 ml of ethyl ether. The precipitate is filtered off, washed with ethyl ether and then dried. The product is taken up in 100 ml of water and brought to pH 6.2 with a saturated aqueous NaHCO₃ solution. After ultrafiltration through a membrane with a cut-off threshold of 1 KD, the retentate is evaporated to dryness and then dried under vacuum. 1.4 g of product are obtained.

HPLC: Supersphere RP Select B® (column; water-TFA, pH 2.80/CH₃CN tr=15.2 min.

Mass spectrum Mode ES⁻ m/z=9223.6 with z=1

### EXAMPLE 20

Compound of formula: E

such that B═

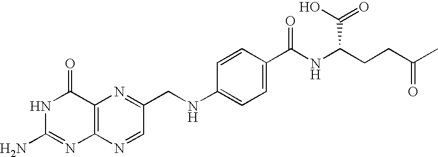

L=as defined in Example 17

HR Ch is such that r is 2 and I_{c} has the formula II′a1 such that:
x=2
-GNH— is 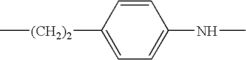
R is 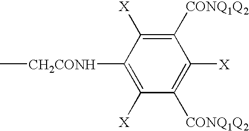
with Q₁=Q₂CH₂(CHOH)₄CH₂OH and X═Br
D is 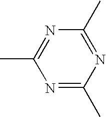
with q=1

with x, y, z=1

0.92 g of intermediate obtained in Example 6 is dissolved in 9 ml of water and then a saturated Na₂CO₃ solution is added in order to obtain a pH=9. 93 mg of the intermediate obtained in Example 11e) are added, along with 5 drops of ethanol. The reaction medium is stirred at ambient temperature for 24 h and the pH is then adjusted to 6.5 with a 1N aqueous HCl solution. The reaction medium is evaporated to dryness under reduced pressure. The product obtained is purified by preparative HPLC. 400 mg of product are obtained.

HPLC: Supersphere RP Select B® column; water-TFA, pH 2.80/CH₃CN; tr=16.6 min.

Mass spectrum: Mode ES⁻ m/z=6987 with z=1

### EXEMPLE 21

Compound of formula: E such that B═

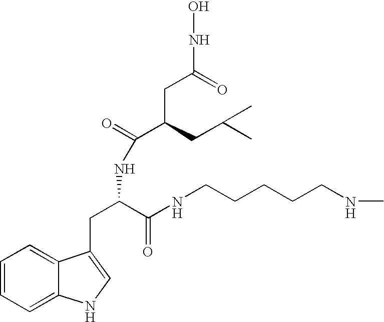

L as defined in Example 19

HR Ch as defined in Example 17

x, y, z=1

1.17 g of the compound obtained in Example 3 are dissolved in 15 ml of H₂O at ambient temperature. The pH of the solution is brought to 9 with an Na₂CO₃ solution. 67.18 mg of the product obtained in stage 14 f) are added, followed by 170 μl of ethanol, and the reaction medium is stirred at ambient temperature, while maintaining the pH at 9 with a saturated Na₂CO₃ solution, for 48 hours. The pH is brought back to 7 with an HCl solution and the solution is precipitated from 150 ml of ethanol. The precipitate is filtered off, washed with 100 ml of ethyl ether, filtered and dried. 1.05 g of product are obtained and purified by preparative HPLC.

HPLC: Symmetry® column; water-TFA, pH 2.80/CH₃CN; tr=13.7 min.

Mass spectrum: Mode ES⁻ m/z=2303.3 with z=4

### EXAMPLE 22

Compound of formula: E

with B═ as defined in Example 20

L as defined in Example 17

HR Ch as defined in Example 17

x, y, z=1.

1.25 g of the compound obtained in Example 3 are dissolved in 50 ml of sodium carbonate (0.1N) at ambient temperature (pH=10). 0.11 g of product obtained in Example 11e) is added, and the reaction medium is stirred at ambient temperature for 24 hours. The mixture is run into 500 ml of ethanol and the precipitate obtained is filtered off and dried under vacuum. The product is purified by preparative HPLC. 0.26 g is isolated.

HPLC: Supersphere RP Select B® column; water-TFA, pH 3/CH₃CN; tr=12.30 min.

Mass spectrum: Mode ES⁻ m/z=1879.3 with z=5

### EXAMPLE 23

### Coupling of Peptides

Compound of formula: E

such that B═ is a peptide among those proposed in the following table:

| Peptide | Sequence | target |
|---|---|---|
| 1 | —NH-Pro-Leu-Gly-NHOH | Matrix |
| | | metallo- |
| | | proteinase |
| 2 | Cyclo(Arg-Gly-Asp-D-Phe-Lys) | αVβ3 |
| 3 | —NH-Val-Cyclo(Cys-Arg-Gly-Asp-Cys)-NH₂ | GP_{IIb}/IIIₐ |
| 4 | —NH-Ado-Ala-Thr-Trp-Leu-Pro-Pro-Arg-NH₂ | VEGF |
| 5 | —NH-Gly-Thr-Lys-Pro-Pro-Arg-COOH | Tuftsin |

L as defined in Example 19

HR Ch as defined in Example 17

x, y, z=1.

The peptides were prepared according to conventional methods from the literature, in liquid phase or on a solid support, using Boc or Fmoc chemistry, manually or by means of an automatic synthesizer.

The peptides used are as follows:

| Peptide | Sequence |
|---|---|
| 1 | Z-NH-Pro-Leu-Gly-NHOH |
| 2 | Cyclo(Arg(Pbf)-Gly-Asp(OtBu)-D-Phe-Lys) |
| 3 | NH₂-Val-Cyclo(Cys-Arg(Pbf)-Gly-Asp(OtBu)-Cys)-NH₂ |
| 4 | NH₂-Ado-Ala-Thr(tBu)-Trp(Boc)-Leu-Pro-Pro-Arg(Pbf)-NH₂ |
| 5 | NH₂-Gly-Thr(tBu)-Lys(Boc)-Pro-Pro-Arg(Pbf)-COOH |

Coupling of Peptide 1 with the Compound of Example 3

Compound of formula: E

such that B═ is —NH-Pro-Leu-Gly-NHOH

L as defined in Example 19

HR Ch as defined in Example 17

x, y, z=1.

a) Debenzylation

1 g of the peptide Z-Pro-Leu-Gly-NHOH (BACHEM®) are dissolved in 100 ml of methanol. 100 mg of palladium-oh-charcoal are added. The entire mixture is placed under hydrogen (40 Psi) at ambient temperature for 8 h (PARR® system). The reaction medium is then filtered through clarcel, concentrated by evaporation under vacuum and then precipitated from ether. 570 mg of white crystals are obtained.

HPLC: Symmetry® C18 column: water-TFA, pH3.20/CH₃CN; tr=5 min.

Mass spectrum: Mode ES+⁻ m/z=301.3 with z=1

b) Coupling

50 mg of the compound obtained in Example 3 are dissolved in 1 ml of water. The pH is brought to 9.5 by adding Na₂CO₃. 5 mg of the peptide obtained according to the preceding stage are added. The reaction medium is stirred at ambient temperature for 48 hours and is then precipitated from ethanol. The product obtained by filtration is then purified by preparative HPLC.

HPLC: Supersphere RP Select B® column; water-TFA, pH 3/CH₃CN; tr=14 min.

Mass spectrum: Mode ES+ m/z=2265 with z=4

Coupling of peptide 2 with the compound of Example 3

Compound of formula: E

such that B═ is cyclo(Arg-Gly-Asp-D-Phe-Lys)

L as defined in Example 19

HR Ch as defined in Example 17

x, y, z=1.

c) Coupling

300 mg of the compound obtained in Example 3 are dissolved in 2 ml of water. The pH is brought to 9.5 by adding Na₂CO₃. 56.3 mg of peptide 2 (Cyclo(Arg(Pbf)-Gly-Asp(OtBu)-D-Phe-Lys)) are added. The reaction medium is stirred at ambient temperature for 3 days and is then precipitated from ethanol.

HPLC: Supersphere RP Select B® column; water-TFA, pH 2.8/H₃CN; tr=7.3 and 19.7.

Mass spectrum: Mode ES⁻ m/z=2416.7 with z=4

d) Deprotection

The compound obtained according to stage c) is dissolved in 10 ml of a trifluoroacetic acid/water/triisopropylsilane (90/5/5) mixture. After 4 h at ambient temperature with stirring, the TFA is eliminated by evaporation under vacuum. The reaction medium is precipited from ether. The product obtained by filtration is then purified by preparative HPLC.

HPLC: Supersphere Select B® column; water-TFA, pH 3/CH₃CN; tr=13.8 min.

Mass spectrum: Mode ES⁻ m/z=2339.3 with z=4

The other compounds are obtained in a similar manner:

Coupling of Peptide 3 with the Compound of Example 3

Starting with peptide 3-(NH₂-Val-Cyclo(Cys-Arg(Pbf-Gly-Asp(OtBu)-Cys)-NH₂) and the compound of Example 3 according to the precedure of stage c) of Example 23.
Deprotection according to the procedure of stage d) of Example 23.
   Coupling of Peptide 4 with the Compound of Example 3:
Starting with peptide 4 (NH₂-Ado-Ala-Thr(tBu)-Trp(Boc)-Leu-Pro-Pro-Arg(Pbf)-NH₂) and the compound of Example 3 according to the procedure of stage c) of Example 23.
Deprotection according to the procedure of stage d) of Example 23.
   Coupling of Peptide 1 with the Compound of Example 10:
Starting with the compound of stage a) of Example 23 and the compound of Example 10 according to the procedure of stage b) of Example 23.
   Coupling of Peptide 2 with the Compound of Example 10:
Starting with peptide 2 (Cyclo(Arg(Pbf)-Gly-Asp(OtBu)-D-Phe-Lys)) and the compound of Example 10 according to the procedure of stage c) of Example 23.
Deprotection according to the procedure of stage d) of Example 23.
   Coupling of peptide 3 with the compound of Example 10:
Starting with peptide 3 (NH₂-Val-Cyclo(Cys-Arg(Pbf)-Gly-Asp(OtBu)-Cys)-NH₂) and the compound of Example 10 according to the procedure of stage c) of Example 23.
Deprotection according to the procedure of stage d) of Example 23.
   Coupling of Peptide 4 with the Compound of Example 10:
Starting with peptide 4 (NH₂-Ado-Ala-Thr(tBu)-Trp(Boc)-Leu-Pro-Pro-Arg(Pbf)-NH₂) and the compound of Example 1.0 according to the procedure of stage c) of Example 23.
Deprotection according to the procedure of stage d) of Example 23.
   Coupling of Peptide 5 with the Compound of Example 3:
Starting with peptide 5 (NH₂-Gly-Thr(tBu)-Lys(Boc)-Pro-Pro-Arg(Pbf)-COOH) and the compound of Example 3 according to the procedure of stage c) of Example 23.
Deprotection according to the procedure of stage d) of Example 23.
   Coupling of Peptide 1 with the Compound of Example 6:
Starting with the compound of stage a) of Example 23 and the compound of Example 6 according to the procedure of stage b) of Example 23.
   Coupling of peptide 2 with the compound of Example 6:
Starting with peptide 2 (Cyclo(Arg(Pbf)-Gly-Asp(OtBu)-D-Phe-Lys)) and the compound of Example 6 according to the procedure of stage c) of Example 23.
Deprotection according to the procedure of stage d) of Example 23.
   Coupling of Peptide 3 with the Compound of Example 6:
Starting with peptide 3 (NH₂-Val-Cyclo(Cys-Arg(Pbf)-Gly-Asp(OtBu)-Cys)-NH₂) and the compound of Example 6 according to the procedure of stage c) of Example 23.
Deprotection according to the procedure of stage d) of Example 23.
   Coupling of Peptide 4 with the Compound of Example 6:
Starting with peptide 4 (NH₂-Ado-Ala-Thr(tBu)-Trp(Boc)-Leu-Pro-Pro-Arg(Pbf)-NH₂) and the compound of Example 6 according to the procedure of stage c) of Example 23.
Deprotection according to the procedure of stage d) of Example 23.

### EXAMPLE 24

Compound of formula: E

such that B═

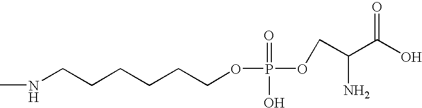

L as defined in Example 19

HR Ch=as defined in Example 17

with x, y, z=1.

a) Condensation

The condensation is carried out starting with the compound of Example 3 and the intermediate obtained in Example 13 c) according to the procedure of Example 19 a).

b) Deprotection

The compound obtained in the preceding stage is dissolved in TFA and treated according to the procedure described in stage 19 b).

### EXAMPLE 25

Compound of formula: E

with B═ as defined in Example 20

L as defined in Example 17

HR Ch is such that r is 2 and Iₑ has the formula II′″1 such that:
x=2
-GNH— is
   —(—CH₂)₃—NH—
R is 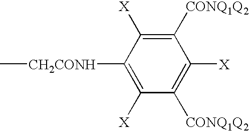
with Q₁=Q₂=CH₂(CHOH)₄CH₂OH and X═Br
D is 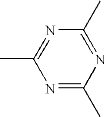
with q=1

with x, y, z=1.

0.650 g of the compound obtained in Example 10 is dissolved in 9 ml of water; the pH brought to 9.2 with Na₂CO₃ and then 0.065 g of compound prepared in Example 11 e) is added along with 0.3 ml of ethanol. The solution is left at ambient temperature for two days and is then run into 90 ml of ethanol. The product obtained is filtered and then dried. After purification by preparative HPLC and ultrafiltration through a membrane with a cut-off threshold of 1 KD; the retentate is concentrated. 200 mg of yellow flakes are obtained.

HPLC: Supersphere RP Select B® column; water-TFA, pH 2.8/CH₃CN; tr=12 min.

Mass spectrum: Mode ES⁻ m/z=2305.6 with z=4

The biological effectiveness of compounds synthesized by the inventors is now described.

### EXAMPLE I

### HR Compounds Using a Folate Receptor-Targeting Agent as Biovector

The inventors in particular tested non-HR biovectors as controls:

a. commerical compounds P853 and P871 which associate a folate and a non-HR DOTA. 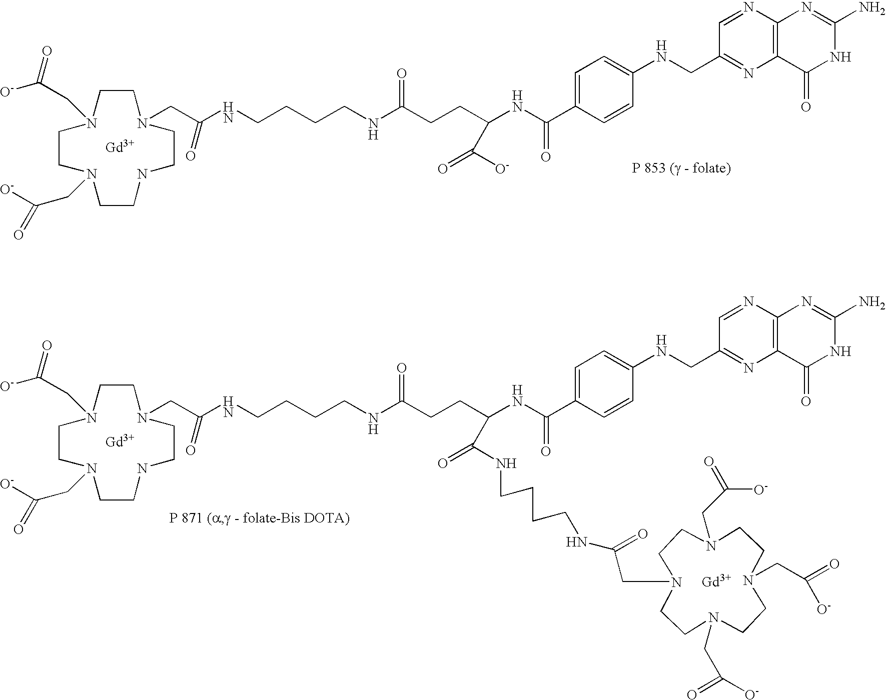
the compound P860 of formula

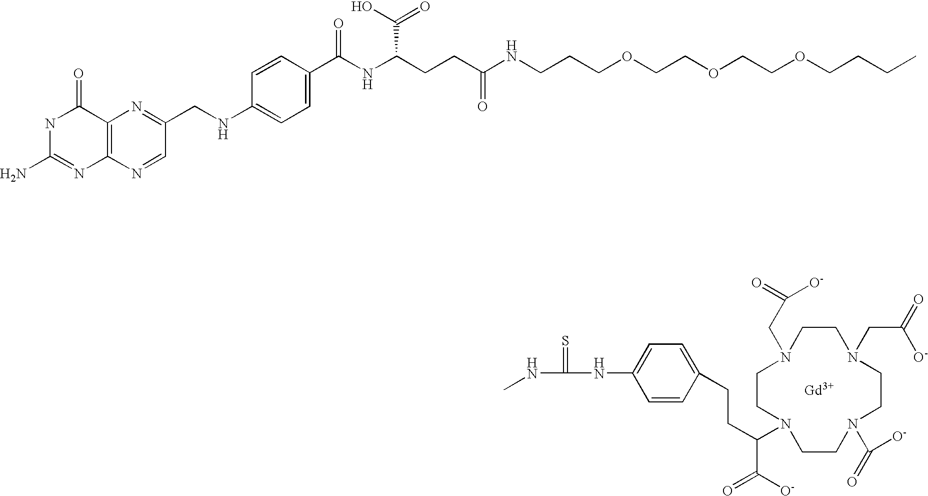

P860 associates a specific folate receptor-targeting biovector and non-HR DOTA, with a PEG-type linker.

The inventors in particular tested as HR biovectors:
the compound BIO-FOLATE.I of formula:

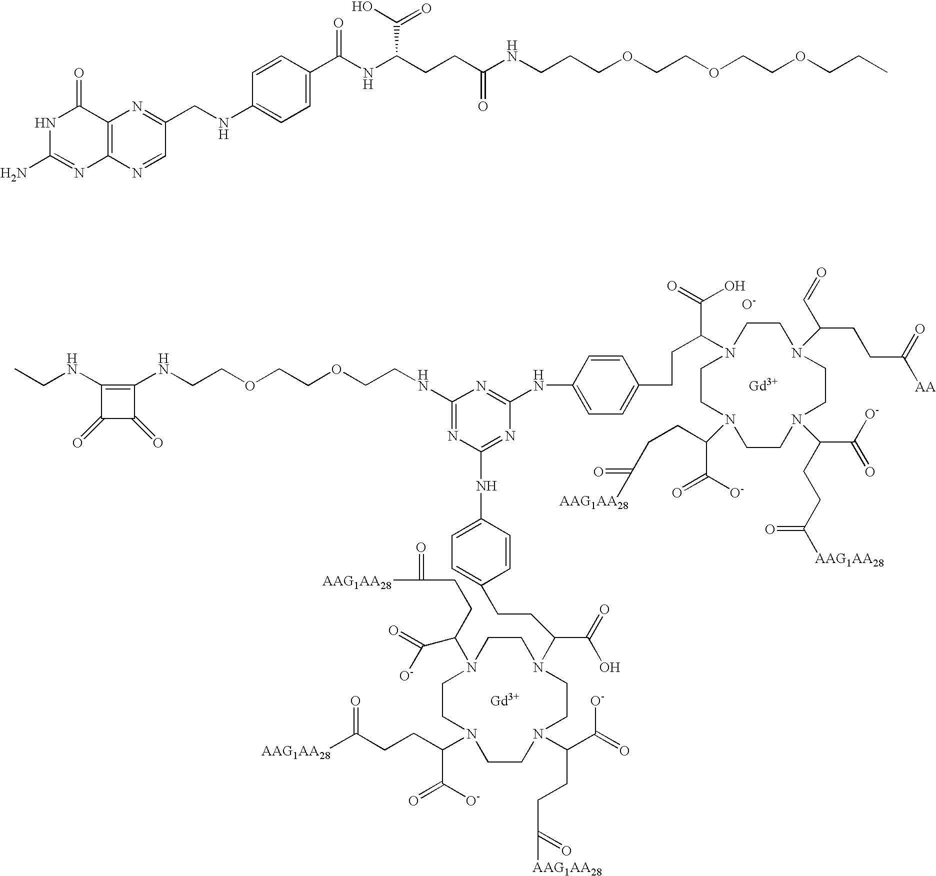

BIO-FOLATE.I associates a specific folate receptor-targeting biovector and an HR DOTA, with a squarate-type linker.

Since BIO-FOLATE.I has 2 Gd per mol of product, the molar relaxivity values are summarized in this table (uncertainty +/−5%).

| | molar r1 | molar r2 |
|---|---|---|
| Frequency | mM⁻¹ · s⁻¹ | mM⁻¹ · s⁻¹ |
| 60 MHz | 53 | 81 |

b. the compound BIO-FOLATE.II of formula

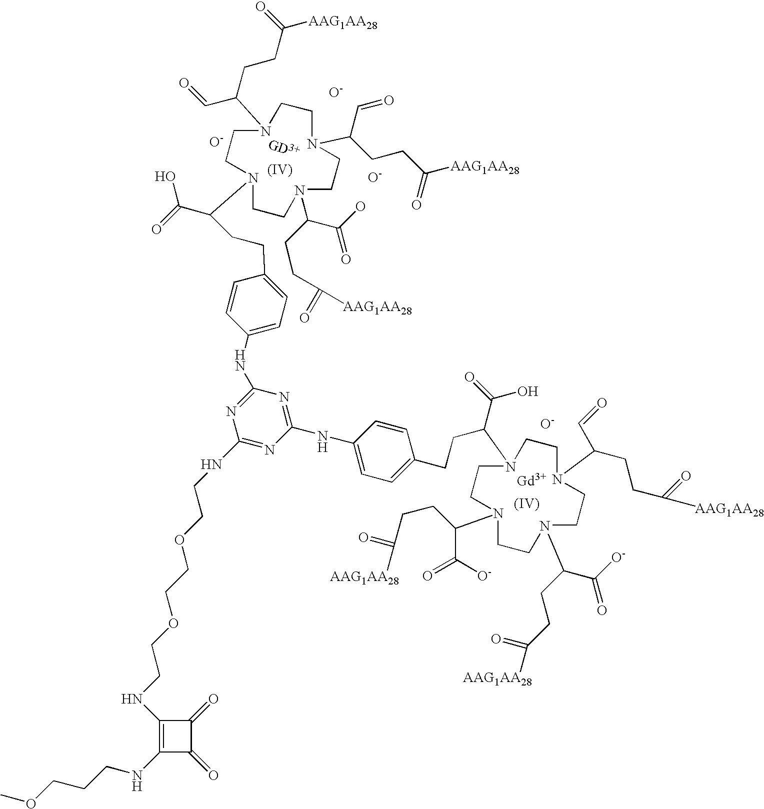

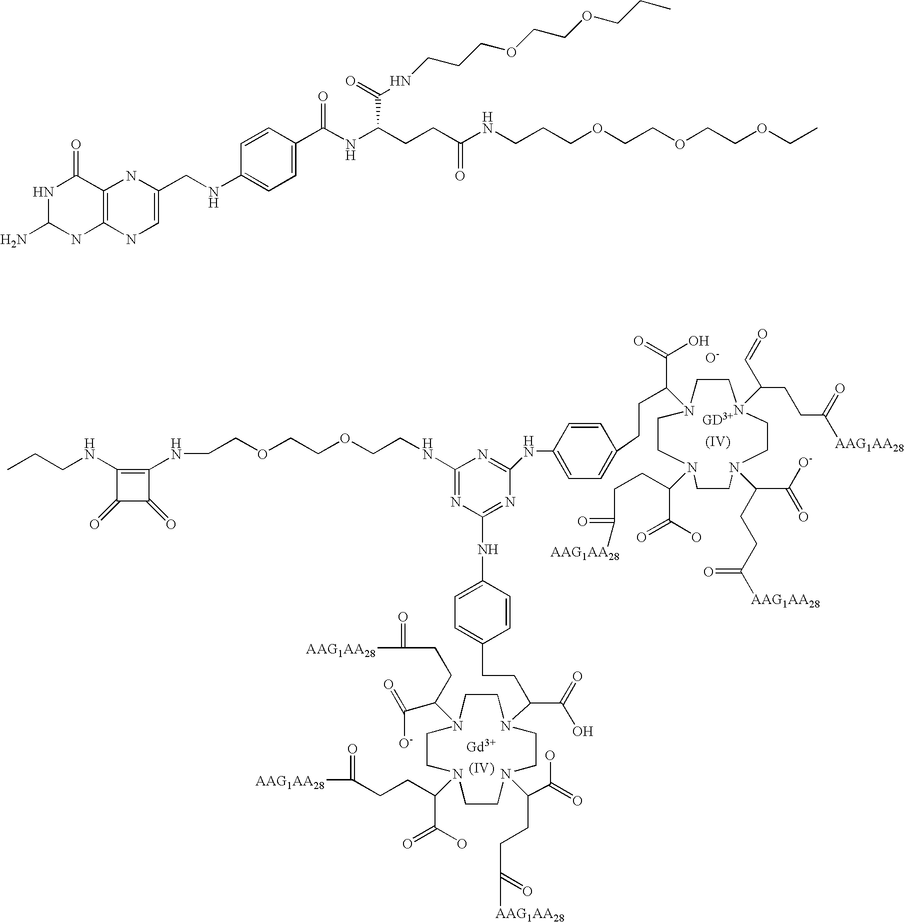

The inventors verified that the control compounds have a high affinity for their receptor in vitro. The binding was tested on an in vitro model of binding to KB cell membranes, in competition with ³H folate.

On the other hand, the in vivo studies clearly demonstrate that P860 is inactive in vivo unlike BIO-FOLATE.I and BIO-FOLATE.II.

c. Biodistribution study on nude mice bearing KB tumours: this biodistribution study comprised competition between P860 and free-folic acid. A group of animals received normal food and a co-injection of folic acid with the product tested and another was given a folate-depleted diet through a single injection of the product tested. This study shows a lack of binding on P860 to FBP.

d. MRI on nude rats bearing KB tumours (study for comparison of P860 and Dotarem on the KB tumour-bearing nude rat model): no visual difference, in terms of the tumour, is observed between the products.

On the other hand, the studies on the product BIO-FOLATE.I give positive results
In vitro study of product binding to FBP (folate binding protein): this study made it possible to show that BIO-FOLATE.I binds specifically to FBP.
In vitro study of binding/internalization in KB cells: this study, carried out at 37° C. with or without excess free folic acid, made it possible to test the amount of product specifically bound and internalized in KB cells, by means of ICP-MS assays. Correct capture of this product at 37° C. is noted, which is in agreement with the preceding, experiments and demonstrates the specificity through the inhibition experiments.
Biodistribution study on nude mice bearing KB tumours The concentration chosen for the BIO-FOLATE.I is 15 μmol/kg, which is within the range of doses which are effective in imaging in humans.

Results: an excellent accumulation of BIO-FOLATE.I in the tumours is observed. A prior injection of free folic at 25 μmol/kg makes it possible to decrease the Gd concentrations assayed in the tumours, showing the specificity of the product.

The molar BIO-FOLATE.I relaxivity in water is very good, in particular at 60 MHz: r1=53 mM⁻¹·s⁻¹. It is recalled that the relaxivity obtained with specific products of the prior art of the type such as dendrimer associated with folates is r1=9.32 mM⁻¹s⁻¹ per Gd (Investigative Radiology, January 2000, vol 35, p 56).
Study of MRI imaging between 30 minutes and 24 hours the contrast at the level of the tumour is very clear, in particular 1 to 2 hours after injection. In addition, Gd assays by ICP-MS; were carried out on the reference organs at the end of the assays using the imager. These experiments show, for example, that BIO-FOLATE.I induces a heightening of the tumours and that this heightening is longer lasting than with products of the prior art. The tumour/muscle ratio is of the order of 4 to 10 times that observed with previously used contrast products.
   Experimental Conditions for the BIACORE 3000 Studies on P860 and BIO-FOLATE.I (Study of Affinity Between P860, BIO-FOLATE.I, and Folate Binding Protein by Means of BIAcore 3000).

Reagents

| Products | Supplier | Reference |
|---|---|---|
| Folic acid | Avogado | 14300 |
| FBP | Sigma | F-0504 |
| CM5 chips | BIAcore | Batch 0374 |

HBS buffer: 10 mM HEPES pH 7.4, 0.15M NaCl, 4.3 mM EDTA and 0.005% NP20

PGM buffer: 100 mM KH₂PO₄, pH 7, 10% glycerol and 4 mM β-mercaptoethanol

The FBP was immobilized at 1 mg/ml in PGM buffer according to the protocols recommended by BIAcore for coupling amines. Next, the remaining active carboxylated groups are saturated with 1M ethanolamine, pH=8.

The folate binding was followed at four different concentrations (125, 250, 500 and 1000 μM). The association and the dissociation were studied with a flow rate at 30 μl/min. The association phase is 5 minutes whereas the dissociation phase is 3 minutes.

Results

| Products | Dissociation rate Kdi |
|---|---|
| Folid acid | 1.55 ± 0.44 10⁻³ s⁻¹ |
| BIO-FOLATE.I | 2.6 ± 1.4 10⁻⁴ s⁻¹ |

Conditions for Biodistribution Study on Nude Mice
Part 1:
Induction of subcutaneous tumours in nude female mice and IV injection of human KB cells.
Separation of the animals into 2 groups receiving a diet with or without folate (10 days).
   The group with folate receives an additional injection of folic acid (25 μmol/kg IV) 5 minutes before the injections of contrast product (CP).
IV injection of CP then removal of organ samples (muscle, tumour, liver, kidneys) and plasma samples.
   Part 2: Assays by ICP-AES
Gd assay (P860, BIO-FOLATE.I, Dotarem): liver, kidney.
   Part 3: Bioanalysis: Assay by ICP-MS
Gd assay (P860, BIO-FOLATE.I, Dotarem): tumour, muscle, plasma

Supplementing with Folate
5.5 mM folic acid diluted in PBS and then injected at a dose of 25 μmol/kg (4.54 ml/kg).

Contrast Products

| | | Concentration | Concentration |
|---|---|---|---|
| | | of Gd | after dilution |
| Product | Vehicle | (mM) | (mM). |
| P860 | 1X PBS | 50 | 44 |
| BIO- | 1X PBS | 10 to 15 | 6.6 |
| FOLATE.I | | | |
| DOTAREM | 1X PBS | 500 | 44 |

Method of Administration

Folic acid and CP: IV administration (caudal vein).

The folic acid was injected 5 minutes before the injection of CP.

Tumour Cells

| Cell line | Type | Origin | Reference |
|---|---|---|---|
| KB | Nasopharyngeal | Human | Taken from a |
| | epidermal carcinoma | | caucasian male adult |
| | | | in 1954 |

KB cells cultured in RPMI 1640 + 10% of SVF.

Induction of Subcutaneous Tumours:
D0: subcutaneous inoculation of 10⁷ cells diluted in 200 μl of RPMI: in the right flank of the animals under gaseous anaesthesia with isoflurane.
D18: 100% of the animals developed a subcutaneous tumour.
   Results: Tumour Distribution of the PC—Gd

Folate-Free Diet
From 14% (at 4 h) to 7% (at 72 h) of the injected theoretical dose of BIO-FOLATE.I is found in the tumours.
In the case of P860, only 1 to 2% of the injected theoretical dose is found in the tumours.
These proportions are conserved when they are weighted by the mass of the tumour sample: respectively, 0.3 to 0.4% for BIO-FOLATE.I versus 0.05 to 0.10% for P860.
For the two CPs, a time-effect is observed between 4 and 72 h, characterized by a gradual decrease in the amounts of CP measured in the tumours.

Competition with Free Folate
Folic acid partially prevents the accumulation of BIO-FOLATE.I in the tumours, at times 4 and 24 h: 2 times less product (5 to 8% of the injected dose) are then found in the tumour.
On the other hand, the distribution of P860 in the tumours is not affected by the presence of free folate.

Tumour Distribution of the Reference CP (DOTAREM)
With the folate-depleted diet, 1 to 2% of the injected theoretical dose of Dotarem is found in the tumours.
As for P860 and BIO-FOLATE.I, a time-effect is observed between 4 and 72 h (decrease in the amount found).
When they are corrected for g of tissue, it is noted that the intratumour amounts of Dotarem become negligable beyond 4 h.

### EXAMPLE II

### HR Compounds Using an MMP Inhibitor as Biovector

DOTAREM (Gd-DOTA salt) is a “non-specific” control product which contains no biovector. Two gadolium-containing compounds, vectorized with an MMP-inhibitor biovector, were tested:
P947 (non-HR-BIOVECTOR compound) which associates a specific MMP-inhibitor biovector and a non-HR DOTA; the compound P947 has the formula: 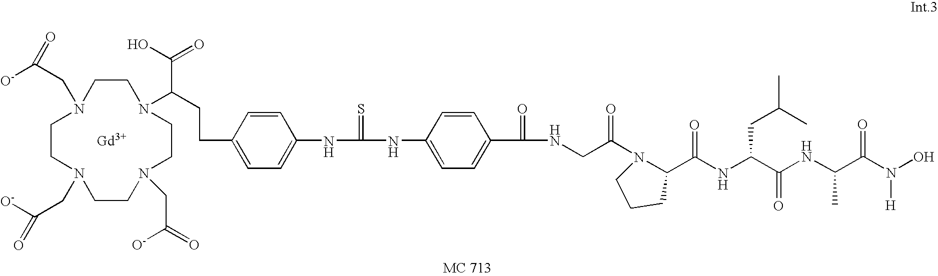
P967 (HR-BIOVECTOR compound) which associates a specific MMP-inhibitor pseudopeptide biovector and an HR-DOTA; the compound P967, unlike P947, contains an HR DOTA.
   In Vitro Assays

The inventors assayed the in vitro activity of the P947 and P967 gadolinium-chelate contrast products functionalized with matrix metalloproteases inhibitors (MMPs), on human MMP-1 and MMP-3. As regards the affinity of the biovector component, on the receptor, the controls were two peptides:

commercial tripeptide: Z-Pro-Leu-Gly-NHOH (Bachem), called peptide A in the study (MMP-1 inhibitor),

commercial tetrapeptide: 4-Abz-Gly-Pro-Dleu-Dala-NHOH (Bachem), called peptide B in the study (inhibitor of MMP-1, MMP-2 and MMP-3).

The inventors verified that the values obtained for the peptides are similar to the values described in the literature, and demonstrated that grafting a peptide onto a Gd probe does not impair the inhibitory activity of the peptide.

More precisely, the in vitro MMP-inhibiting activity was evaluated in the following way:
1. Incubation of the test product in the presence of the MMP enzyme, at 37° C., for a defined period of time, and then measurement of the fluorescence of the medium (t=0).
2. Induction of the enzyme reaction by addition of the substrate to (which becomes fluorescent when it is cleaved by the enzyme), and then incubation at 37° C. for 40 min. A second measurement of fluorescence is then taken (t=40).
3. The activity of the MMP enzyme is determined by subtraction of the signal measured by fluorimetry: (t=40)-(t=0).
4. The results are expressed as percentage inhibition of the activity of the control enzyme.
5. The standard inhibitory product is TIMP-1.

The products were tested in duplicate, at 10⁻⁵, 10⁻⁷ and 10⁻⁹ M with respect to peptides. Those which contain no peptides were treated in a manner similar to the corresponding functionalized contrast products.

MMP-1:

| | | SIGNIFICANT | IC50 OF THE |
|---|---|---|---|
| | CONCENTRATION | INHIBITORY | PEPTIDE |
| PRODUCTS | OF PEPTIDE | EFFECT | (LITERATURE) |
| PEPTIDE A | >10⁻⁵M | N.D. | 4 × 10⁻⁵M |
| PEPTIDE B | 10⁻⁵M | 46% | 10⁻⁶M |
| P947 | 10⁻⁵M | 86% | 10⁻⁶M |
| P967 | 10⁻⁶ to 10⁻¹⁰M | >50% | 10⁻⁶ to 10⁻¹⁰M |

The inventors also tested the in vitro activity in order to evaluate the inhibitory activity of P947 and of the tetrapeptide on MMP-2, a gelatinase A expressed constitutively in the vascular wall and overexpressed in the case of inflammation.

Control: commercial tetrapeptide (called peptide B in the study): 4-Abz-Gly-Pro-Dleu-Dala-NHOH (Bachem); inhibitor of MMP-1, MMP-2 and MMP-3. Experimentally, the tetrapeptide has significant inhibitory activity on MMP-2 from 10⁻⁵ M; this result is in agreement with the data from the literature (IC₅₀ of the commercial tetrapeptide on MMP-2=3×10⁻⁵ M). In addition, grafting of the tetrapeptide onto a gadolinium-containing chelate does not change its effect with respect to MMP-2, since similar results are obtained with P947.

In conclusion regarding the in vitro assays, the known compounds of the prior art (biovector associated with non-HR chelate) and the HR compounds obtained by the inventors show a conserved specificity.

The detailed protocol is as follows for MMP2s: the product tested is added to a 50 mM. Tris-HCl buffer (pH 7.5) containing 150 mM NaCl, 10 mM NaCl₂, 0.02% NaN₃, 0.05% Brij®35 and 0.35 μM MMP-2 activated by incubation for 120 minutes at 37° C. with 6.67 mM APMA. The fluorescence intensity is measured after preincubation for 30 minutes at 37° C., at lambda ex=340 nm and lambda em=405 nm. The enzyme reaction is initiated by adding 6 μM NFF-2, with subsequent incubation for 90 minutes at 37° C.

III) HR Compounds Using Phosphatidylserine as Biovector

III.1 In Vitro Studies

In vitro validation was performed by comparing the rate of uptake by activated macrophages versus non-activated macrophages (activated THP-1 cells are positive for the PS receptor). This study made it possible to demonstrate the interaction of fluorescent phosphatidylserine-NBD (PS-NBD) with the THP-1 line. PS-NBD is a phosphatidylserine comprising only one fatty chain, the other arm linked to the serine head supporting the fluorescence marker NBD.

The THP-1 cell line is a human monocyte line which, after activation, differentiates into macrophages and expresses the phosphatidylserine receptor. These cells were cultured in the presence of phosphatidylserine-NBD.

PS-NBD: 810192 Avanti Polar-Lipids (COGER)

Protocol with Flow Cytometry
2 ml of a suspension of THP-1 at 10⁶ C/ml are seeded in a 6-well plate.
The THP-1 are activated for 24 hours with 50 nmol/l of PMA.
700 ml of each of the dilutions (from the solution at 6.25 mmol/l) are incubated for 24 hours.
The excess product is suctioned off.
The cell layer is washed with 1 ml of RPMI without phenol red.
The excess is suctioned off.
The cells are resuspended in 1 ml of PBS (detachment of the cells using a flow of medium over the layer then scraping of the cells which have not detached, using a scraper).
The cells are kept in the dark at 4° C. while awaiting reading.
The fluorescence is read in FL1 at 525 nm by flow cytometry.

PS-NBD range tested: 13, 32 and 64 μM

Controls: THP-1

Groups tested: THP-1/PMA, THP-1/PS-NBD at 5 μM.

Results: the ratio of the negative control to the highest concentration is greater than a factor of 50.

III.2 Ex Vivo Study

The ex vivo results show an interaction between primary macrophages from WHHL rabbits and the phosphatidylserine-NBE) (PS-NBD). In fact macrophages were isolated from aortic arch and mesenteric lymph node samples, and then incubated for 18 h ex vivo in the presence of PS-NBD. Analysis of these cells by fluorescence microscopy showed a large intracellular accumulation of PS-NB. More precisely, the rabbit was subjected to exsanguinating perfusion with heparinized newborn calf serum before being sacrificed. The aortic arch, the thoracic aorta and the abdominal aorta were removed. The tissue removed were incubated for 18 hours in cell culture medium to which PS-NBD had been added and were then mounted in frozen blocks in order for histological sections to be cut. Certain sections were observed by fluorescence microscopy and others were immunolabelled using an anti-rabbit macrophage antibody (RAM 11).

Incubation of the Tissues with PS-NBD

Incubation of the samples at 37° C. in 24-well plates in the presence of PS-NBD at 35 μM for 18 hours.
2 rinses of the arteries in PBS.
Embedding of each piece of aorta in a freezing gel.
Cryofixation.
Storage of the components in a −80° C. freezer.
   Immunohistochemistry (HEGP)
The mouse primary antibody Ram 11 is used at a 1/100 dilution.
The secondary antibody (goat anti-mouse) is coupled to alkaline phosphatase.
The chromogen used is ADC (supplied by AbCys).
Fast re counterstaining is carried out.

Macrophages were demonstrated in all the sections by immunohistochemistry.

The inventors also demonstrated, ex-vivo, the distribution of the PS-NBD in atheroma plaques of ApoE-KO mice. For this, samples of heart comprising valves and aortic arch and also the iliac bifurcation are incubated in the presence of PS-NBD. This study made it possible to demonstrate that phosphatidylserine interacts specifically with the macrophages located in the plaques.

The inventors also demonstrated (comparative study between negative KB cells and positive THP1 cells) that the recognition of PS-NBD by the phosphatidylserine receptor expressed at the surface of the activated macrophages effectively involves the serine phosphate (polar group) and not the fatty chains. For this, the same assays were carried out, firstly with PS-NBD and, secondly, with NBD-PS (fluorescence marker linked to the serine head and not to the fatty chains), both ex-vivo and in vitro (on activated THP1 cells).

In vitro, on THP1 cells (activated versus non-activated), the inventors tested a compound of formula (E) with B being a PS derivative, the HR chelate being identical to that used for BIO-FOLATE.I, and showed significantly greater binding and/or accumulation in the positive activated THP1 group.

1. Compounds of general formula (E) below:
Bₓ-L_{z}-(HRCh)_{y} (E)
in which:
B is a biovector
L is a linker
HR Ch represents a chelate of formula (I):
[(D)_{q}-(I_{a,b,c,d,e,f,g})ᵣ];
with:
a) I_{a,b,c,d,e,f,g} chosen from Iₐ, I_{b}, I_{c}, I_{d}, Iₑ, I_{f}, I_{g},
Iₐ, I_{b}, I_{c} having the meanings: 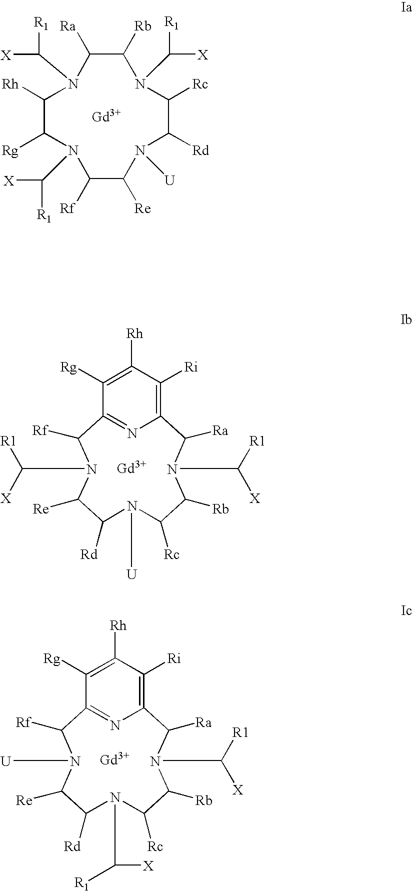
where:
the X, which may be identical or different, are chosen from CO₂R′ₐ, CONR′_{b}R′_{c} or P(R′_{d})O₂H, with:
R′ₐ, R′_{b} and R′_{c}, which may be identical or different, representing H or (C₁-C₈) alkyl, which is optionally hydroxylated;
P is the phosphorus atom, R′_{d} is chosen from OH, (C₁-C₈)alkyl or (C₁-C₈)alkoxy, (C₁-C₈)arylalkyl or (C₁-C₈)alkoxyalkyl;
R1 represents a hydrophilic group of molecular weight greater than 200, selected from groups
polyoxy(C₂-C₃)alkylene,
polyhydroxyalkyl
polyol
(R₂g)ₑ[(R₂g)ᵢR₃]ₕ where:
h=1 or 2; i=0, 1 or 2; e=1 to 5
R₂ represents (the R₂ being identical or different)
nothing, an alkylene, an alkoxyalkylene, a polyalkoxyalkylene;
a phenylene, or a heterocyclic residue which may be saturated or unsaturated, optionally substituted with OH, Cl, Br, I, (C₁-C₈)alkyl, (C₁-C₈)alkyloxy, NO2, NR_{X}R_{Y}, NR_{X}COR_{Y}, CONR_{X}R_{Y} or COOR_{X}, R_{X} and R_{Y} being H or (C₁-C₈)alkyl, and the linear, branched or cyclic C₁-C₁₄ alkyl, alkylene and alkoxy groups possibly being hydroxylated;
g represents (the g being identical or different): nothing or a function O, CO, OCO, COO, SO3, OSO2, CONR′, NR′CO, NR′COO, OCONR′,NR′, NR′CS, CSNR′, SO₂NR′, NR′SO₂, NR′CSO, OCSNR′,NR′CSNR′, P(O)(OH)NR′, NR′P(O)—(OH), in which R′ is H, (C₁-C₈)alkyl or R₃;
R₃ represents alkyl, phenyl, alkyl substituted or interrupted with one or more phenyl groups, alkyleneoxy groups; amino or amido unsubstituted or substituted with alkyl optionally substituted or interrupted with one of the above groups; phenyl, phenylene and heterocyclic groups which may be substituted with OH, Cl, Br, I, (C₁-C₈)alkyl, (C₁-C₈)alkyloxy, NO2, NR_{X}R_{Y}, NR_{X}COR_{Y}, CONR_{X}R_{Y} or COOR_{X}, R_{X} and R_{Y} being H or (C₁-C₈)alkyl, and linear, branched or cyclic C₁-C₁₄ alkyl, alkylene and alkoxy groups which may be hydroxylated;
Rₐ to Rᵢ independently represent H, alkyl, hydroxyalkyl, alkylphenyl or cycloalkyl.
U is a group —CXR₄-linker 1, CHR₄CON-linker 1, CHR₄—CHR₅OH-linker 1
R₄ and R₅ independently representing H, alkyl or hydroxyalkyl,
X having the meaning above,
linker 1 being the linker providing the link between a chelate I_{a, b, c}, and the linker L when q=0 and between I_{a, b, c}, and D when q=1
I_{d}, Iₑ, I_{f} having the meanings 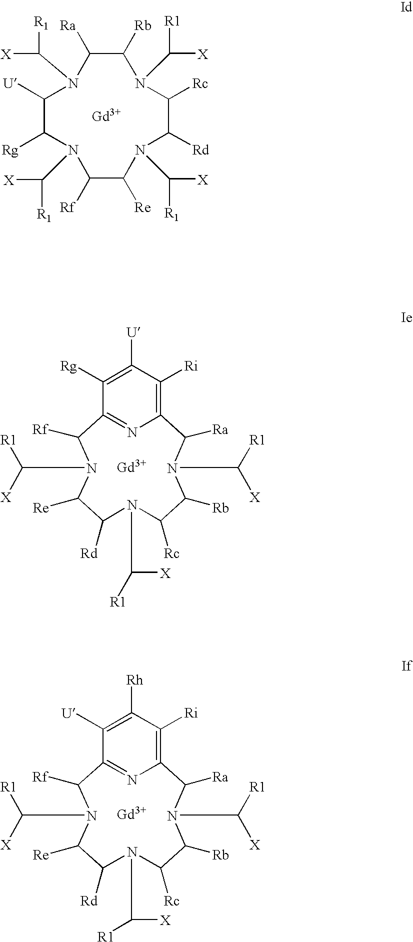
X, R1, Ra to Ri having the same meaning as above,
U′ is linker 1, providing the link between a chelate I_{d,e,f} and a linker L when q=0 and between I_{d,e,f} and D when q=1,
I_{g} representing 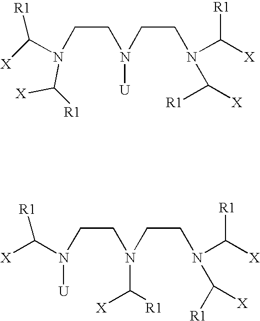
U, X, R1 having the same meaning as above, linker 1 providing the link between a chelate I_{g} and a linker L when q=0 and between I_{g} and D when q=1.
b)
q=0 or q=1
r=1 when q=0, or r is between 2 and 5 when q=1
c) D is a polyfunctional molecule capable of linking a linker L to at least two chelates I_{a,b,c,d,e,f,g}
d) x, y and z are between 1 and 8, preferably x=1 to 3, y=1 to 6, z=1 to 3, given that y=z;
and also the salts of the compounds of formula (E) with pharmaceutically acceptable inorganic or organic acids or bases.

2. Compound according to claim 1, wherein R1 is (CH₂)ₓCONHR with x=1, 2 or 3 and R is a hydrophilic group of molecular weight greater than 200, chosen from:
1) a group: 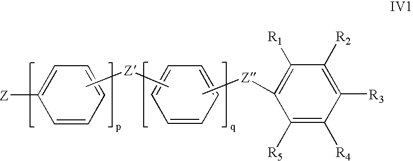
and Z is a bond, CH₂, CH₂CONH or (CH₂)₂NHCO
Z′ is a bond, O, S, NQ, CH₂, CO, CONQ, NQCO, NQ-CONQ or CONQCH₂CONQ,
Z″ is a bond, CONQ, NQCO or CONQCH₂CONQ
p and q are integers, the sum of which is 0 to 3;
R₁, R₂, R₃, R₄ or R₅ represent:
either, independently of one another, H, Br, Cl, I, CONQ₁Q₂ or NQ₁COQ₂ with Q₁ and Q₂, which may be identical or different, being H or a (C₁-C₈)alkyl group which is mono- or polyhydroxylated or optionally interrupted with one or more oxygen atoms, and at least one and no more than two of R₁ to R₅ are CONQ₁Q₂ or NQ₁COQ₂;
or R₂ and R₄ represent 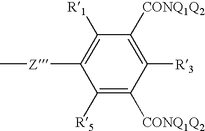
and R₁, R′₁, R₃, R′₃, R₅ and R′₅, which may be identical or different, represent H, Br, Cl or I, Q₁ and Q₂ have the same meaning as above and Z′″ is a group chosen from CONQ, CONQCH₂CONQ, CONQCH₂, NQCONQ and CONQ(CH₂)₂NQCO and Q is H or (C₁-C₄)alkyl, which is optionally hydroxylated, it being possible for the alkyl groups to be linear or branched;
2) a “flash” branch 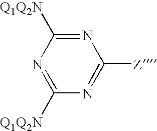
with Z″″ being NQ(CH₂)ⱼ(CH₂OCH₂)ᵢ(CH₂)ⱼNH₂ with i=2 to 6 and j=1 to 6

3. Compound according to claim 1, wherein q=1.

4. Compound according to claim 1, wherein HR Ch represents the group: 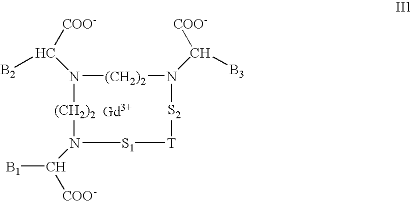
in which:
—S₁-T-S₂— is
1) either 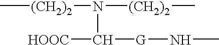
where S₁═S₂═(CH₂)₂
with all three of B₁, B₂ and B₃ representing (CH₂)ₓCONHR with x=1, 2 or 3
2) or 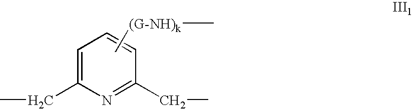
with k=0 and S═S₂═CH₂
one of B1, B2, B3 representing G-NH, and the others representing (CH₂)ₓCONHR
3) or 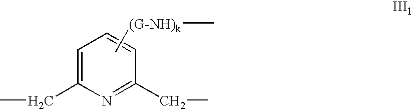
with k=1
all three of B₁, B₂, B₃ representing (CH₂)ₓCONHR with x=1, 2 or 3
and GNH chosen from:
the groups —(CH₂)ₙ—NH— with n=1 to 4,
or

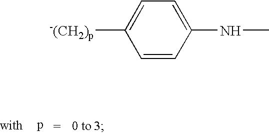

5. Compound according to claim 3, wherein HR Ch represents a group chosen from:
1) the group 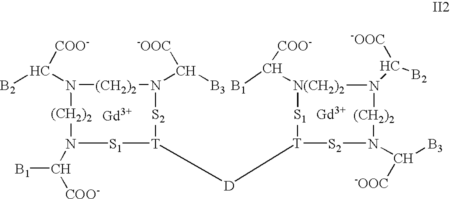
in which
—S₁-T-S₂— is 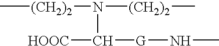
where S₁═S₂═(CH₂)₂
all three of B₁, B₂, B₃ representing (CH₂)ₓCONHR with x=1, 2 or 3
2) the group 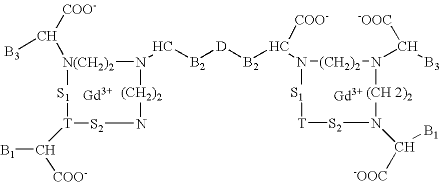
IIa2 (compound referred to as N-functionalized PCTA)
or IIb2 (compound referred to as N-functionalized PCTA and positional isomer of IIb2) 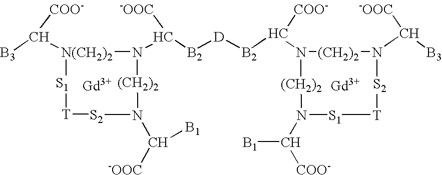
IIb2
in which S₁-T-S₂— is: 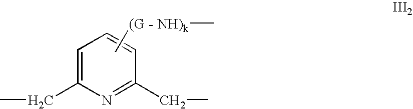
with k=0 and S═S₂═CH₂;
B₃ representing G-NH, and B1 and B2 representing (CH₂)ₓCONHR for IIa2
B₂ representing G-NH, and B1 and B3 representing (CH₂)ₓCONHR for IIb2
3) the group 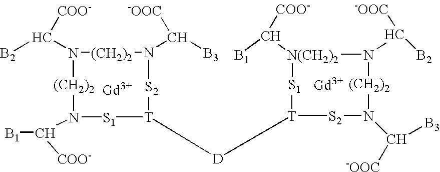
IIc2 (compound referred to as C-functionalized PCTA)
when S₁-T-S₂— is: 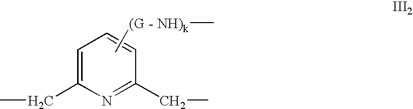
with k=1 and S═S₂═CH₂;
all three of B1, B₂, B3 representing (CH₂)ₓCONHR with x=1, 2 or 3 for IIc2
given that, for II2, IIa2, IIb2 and IIc2,
GNH is chosen from the groups —(CH₂), —NH— with n=1 to 4,
or 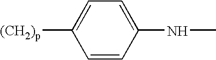
with p=0 to 3;

6. Compound according to claim 1, wherein D is an aromatic backbone polyfunctionalized with carboxylate and/or amino groups

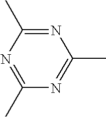

7. Compound according to claim 1, wherein L is a linker chosen from polyoxyalkylenes, squaric acid, a squarate-PEG radical, an alkylene, alkoxyalkylene, polyalkoxyalkylene, alkylene interrupted with phenylene, alkylidene, alkilidene.

8. Compound according to claim 2, in which x of (CH₂)xCONHR is 2 and q=1.

9. Compound according to 4, in which —S₁-T-S₂— represents: 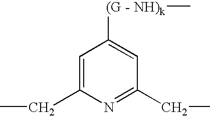
with S₁═S₂═CH₂.

10. Compounds according to claim 9 of formula III1 in which k is 1 and G is —(CH₂)₃—.

11. Compounds according to claim 9 of formula III1 in which k is 0 and B₂ or B₃ represents —(—CH₂)₃NH— or

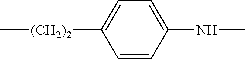

12. Compound according to claim 4, in which —S₁-T-S₂— represents: 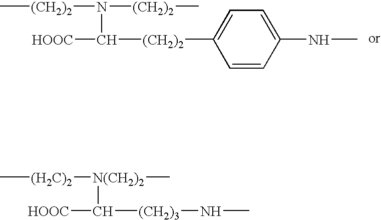
with S₁═S₂═(CH₂)₂.

13. Compounds according to claim 4, for which B₁, B₂ and B₃, when they do not represent -G-NH, represent —(CH₂)₂CONHR, with, in R, p=q=0 and Z being —CH₂CONH.

14. Compounds according to claim 13, for which R represents: 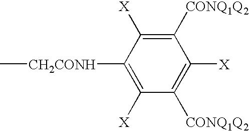
and the X are identical and represent Br or I, while Q₁ and Q₂, which may be identical or different, are mono- or polyhydroxylated (C₁-C₈)alkyl groups such that each CONQ₁Q₂ contains from 4 to 10 hydroxyls in total.

15. Compounds according to claim 13, for which R represents: 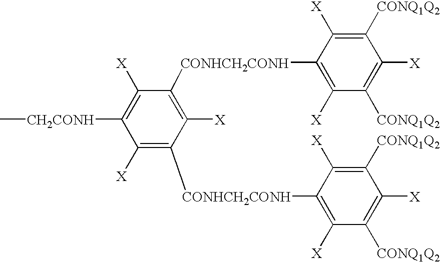
and the X, which are identical, are Br or I, and Q₁ and Q₂, which may be identical or different, are mono- or polyhydroxylated (C₁-C₈)alkyl groups such that each CONQ₁Q₂ group contains from 4 to 10 hydroxyls in total.

16. Compounds according to claim 2, for which R represents: 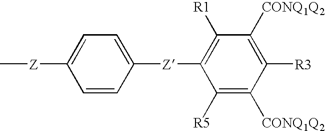
Z is CH₂ or CH₂CONH, Z′ is CONH or CONHCH₂CONH, R₁, R₃ and R₅, which are identical, are Br or I, and Q₁ and Q₂, which may be identical or different, are mono- or polyhydroxylated (C₁-C₈)alkyl groups such that each CONQ₁Q₂ group contains from 4 to 10 hydroxyls in total.

17. Compounds according to claim 2, for which R represents: 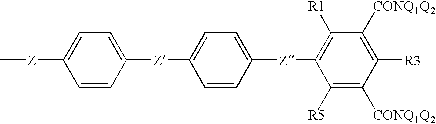
Z is CH₂CONH, Z′ is CONH, Z″ is CONHCH₂CONH and R₁, R₃ and R₅, which are identical, are Br or I, and Q₁ and Q₂, which may be identical or different, are monohydroxylated or polyhydroxylated (C₁-C₈)alkyl groups such that each CONQ₁Q₂ group comprises from 4 to 10 hydroxyls in total.

18. Compounds according to claim 2, for which R represents 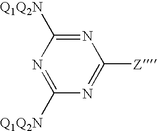
with Z″″ being NQ(CH₂)ⱼ(CH₂OCH₂)ᵢ(CH₂)ⱼNH₂, with i=2 to 6 and j=1 to 6,

19. Compound according to claim 1, wherein the biovector is an agent capable of targeting cellular receptors or tissue components.

20. Compound according to claim 1, wherein the biovector is an agent capable of targeting a folate receptor, (E) being written: 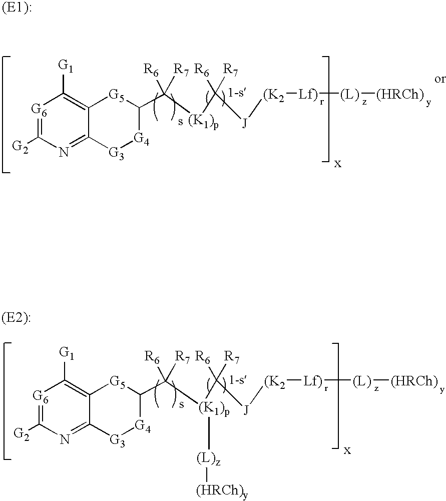
with:
a) G1 is chosen independently from the group consisting of: halo, R_{f}2, OR_{f}2, SR_{f}3, N R_{f}4 R_{f}5;
b) G2 is chosen independently from the group consisting of: halo, R_{f}2, OR_{f}2, SR_{f}3, and N R_{f}4 R_{f}5;
c) G3 and G4 represent divalent groups chosen independently from the group consisting of —(R_{f}6′) C═, —N═, —(R_{f}6′)C(R_{f}7′)—, —N(R_{f}4′)—;
d) G5 is absent or chosen from —(R_{f}6′) C═, —N═, —(R_{f}6′)C(R_{f}7′)—, —N(R_{f}4′)—;
e) the ring J is a possibly heterocyclic aromatic 5- or 6-membered ring, it being possible for the atoms of the ring to be C, N, O, S;
f) G6 is N or C;
g) K1 and K2 are chosen independently from the group consisting of —C(Z_{f})—, —C(Z_{f})O, —OC(Z_{f})—, —N(R_{f}4″)—, —C(Z_{f})—N(R_{f}4″), —N(R_{f}4″)—C(Z_{f}), —O—C(Z)—N(R_{f}4″)—, —N(R_{f}4″)—C(Z_{f})—O—, N(R_{f}4″)—C(Z_{f})—N(R_{f}5″)—, —O—, —S—, —S(O)—, —S(O)₂—, —N(R_{f}4″)S(O)₂—, —C(R_{f}6″)(R_{f}7″)—, —N(C≡CH)—, —N(CH₂—C≡CH)—, C₁-C₁₂ alkyl and C₁-C₁₂ alkoxy; in which Zf is O or S; K2 possibly being covalently bonded to an amino acid;
h) R_{f}1 is chosen from the group consisting of: H, halo, C₁-C₁₂ alkyl and C₁-C₁₂ alkoxy; R_{f}2, R_{f}3, R_{f}4, R_{f}4′, R_{f}4″, R_{f}5, R_{f}5′″, R_{f}6″ and R_{f}7″ are chosen independently from the group consisting of: H, halo, C₁-C₁₂ alkyl, C₁-C₁₂ alkoxy, C,-C, 2 alkanoyl, C,-C, 2 alkenyl, C₁-C₁₂ alkynyle, (C,-C, 2 alkoxy)carbonyl and (C₁C₁₂ alkylamino)carbonyl;
i) R_{f}6 and R_{f}7 are chosen independently from the group consisting of: H, halo, C₁-C₁₂alkyl, C₁-C₁₂ alkoxy; or R_{f}6 and R_{f}7 together form O═;
j) R_{f}6′ and R_{f}7′ are chosen independently from the group consisting of: H, halo, C₁-C₁₂ alkyl, C₁-C₁₂ alkoxy; or R_{f}6′ and R_{f}7′ together form O═;
k) L_{f} is a divalent linker which includes, where appropriate, a natural amino acid or a natural poly(amino acid), this acid or polyacid being bonded to K2 or to K1 via its alpha-amino group via an amide bond;
l) n, p, r and s are independently 0 or 1.

21. Compound according to claim 20, wherein G1 is NH₂ or OH.

22. Compound according to claim 20, wherein G3 is —N═ or —CH— when the ring comprising G3 is aromatic, and G3 is —NH— or —CH₂— when the ring comprising G3 is non-aromatic.

23. Compound according to claim 20, wherein G4 is —CH— or —C(CH₃)— when the ring comprising G3 is aromatic, and —CH₂— or —CH(CH₃)— when the ring comprising G3 is non-aromatic.

24. Compound according to claim 20, wherein G5 is absent.

25. Compound according to claim 20, wherein that G6 is N or C.

26. Compound according to claim 20, wherein (E) is 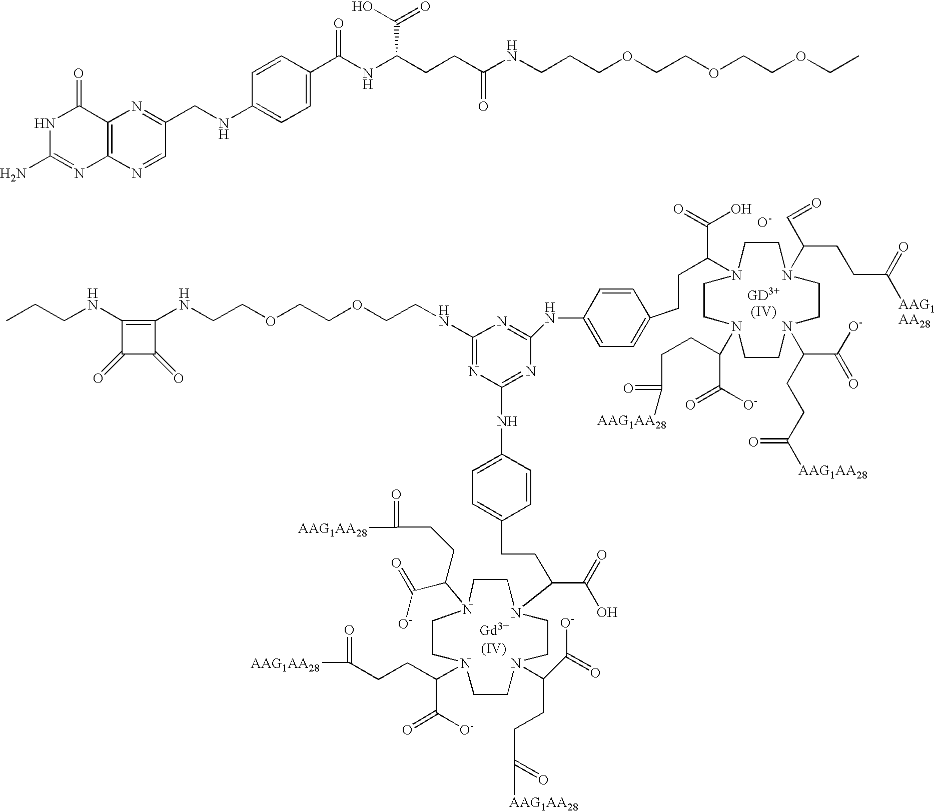
or

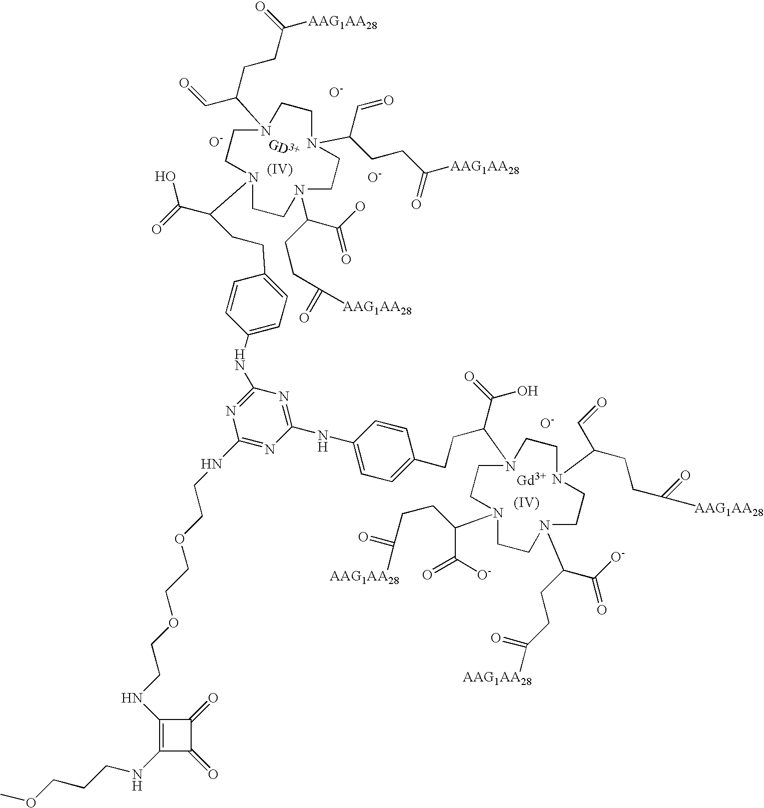

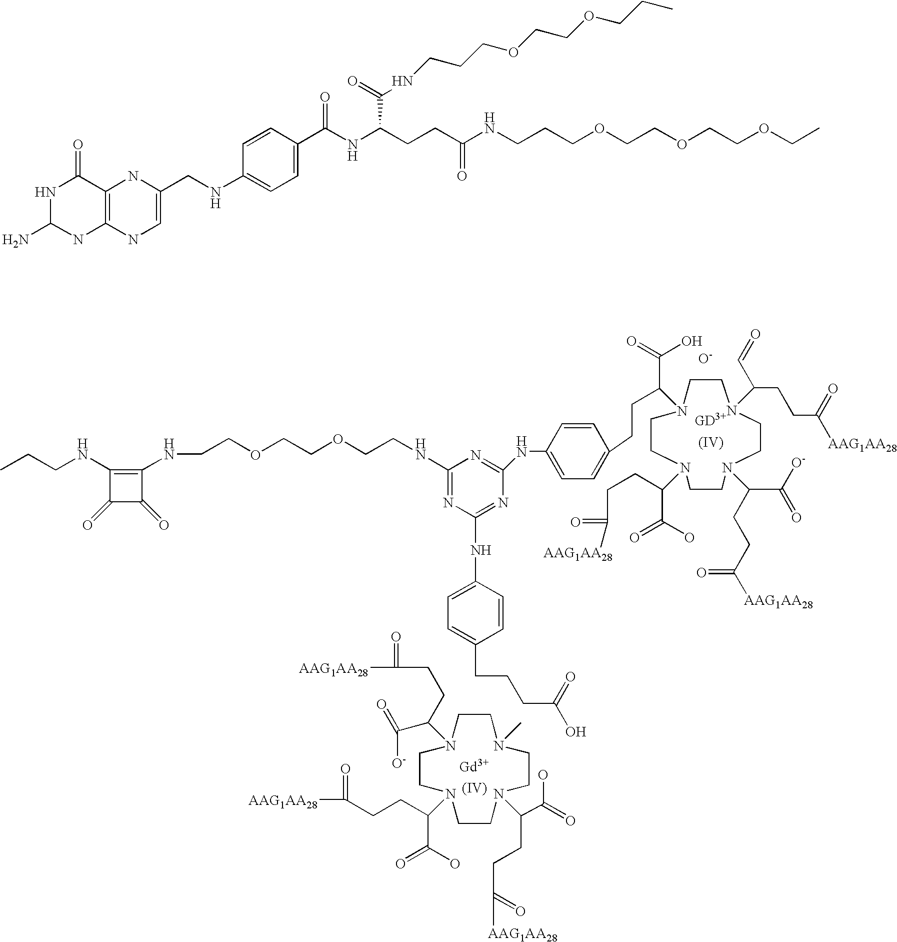

27. Compound according to claim 1, wherein the biovector is an angiogenesis inhibitor.

28. Compound according to claim 1, wherein the biovector is an agent capable of inhibiting the activity of an MMP.

29. Compound according to claim 28, wherein the biovector is an MMP inhibitor derived from ilomastat.

30. Compound according to claim 1, wherein the biovector is an agent capable of targeting an integrin.

31. Compound according to claim 30, wherein the biovector is an agent capable of targeting the integrin αvβ3.

32. Compound according to claim 31, wherein the biovector is an RGDfV peptide having the structure

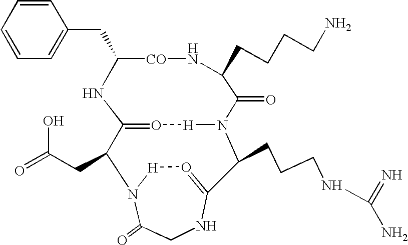

33. Compound according to claim 30, wherein the biovector is an agent capable of targeting the integrin GPIIb/IIIa.

34. Compound according to claim 30, wherein the biovector is an agent capable of targeting a vitronectin.

35. Compound according to claim 1, wherein the biovector is an agent capable of targeting an angiogenic receptor of endothelial cells.

36. Compound according to claim 1, wherein the biovector is an agent capable of targeting receptors located on macrophages.

37. Compound according to claim 36, wherein the biovector is a derivative of phosphatidylserine.

38. Compound according to claim 1, wherein the biovector is a bisphosphonate derivative.

39. Compound according to of claim 1, wherein the biovector is a peptide targeting tuftsin.

40. Compound according to claim 1, wherein the biovector is Annexin 5.

41. Intermediate compound, used for preparing a compound according to claim 1, of formula:
L-[(D)_{q}-(I_{a,b,c,d,e,f,g})ᵣ]
with q=1

42. Compound according to claim 1, in its form bonded to an element M, (E) being written Bₓ-L-(HR Ch)_{y}-M; given that M is either a paramagnetic metal ion having the atomic number 21-29, 42-44, or 58-70, or a radionucleide, or a heavy metal ion having the atomic number 21-31, 39-49, 50, 56-80, 82, 83 or 90.

43. Magnetic resonance imaging contrast product, wherein it comprises a compound according to claim 1 optionally combined with a pharmaceutically acceptable vehicle.

44. Contrast product according to claim 43, provided in the form of an injectable sterile solution.

45. (canceled)

46. Nuclear medicine product, wherein it comprises a compound according to claim 1, optionally combined with a pharmaceutically acceptable vehicle.

47. Compound according to claim 1, having a relaxivity of between 25 and 200 mM⁻¹Gd⁻¹.

48. Method for preparing a compound according to claim 1, wherein it comprises the coupling of at least one biovector and at least one high-relaxivity chelate as defined in claim 1.

49. Compound according to claim 2, wherein R represents 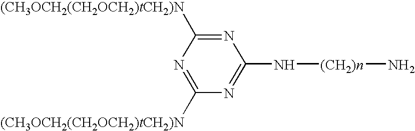
or 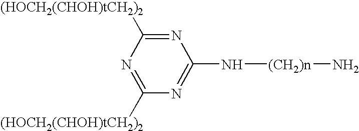
with t=1, 2, 3 or 4 and n=2 to 6.

50. Compound according to claim 6, wherein D is of 1,3,5-triazine type, of formula:
linker 2 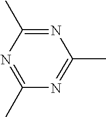
with linker 2 chosen from a) and b) and preferably a)
c) (CH₂)₂-φ-NH₂, (CH₂)₃—NH₂, NH—(CH₂)₂—NH, NH—(CH₂)₃—NH,
d) P1-I-P2, which may be identical or different, P1 and P2 being chosen from OH, SH, NH₂, nothing, CO₂H, NCS, NCO, SO₃H,
with 1=alkylene, alkoxyalkylene, polyalkoxyalkylene, alkylene interrupted with phenylene, alkylidene, alkilidene.

51. Compound according to claim 6, wherein D is 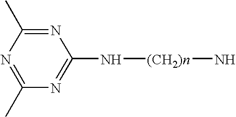
or

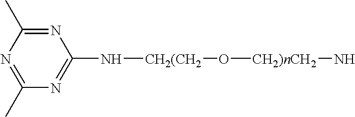

52. Compound according to claim 18, wherein R represents 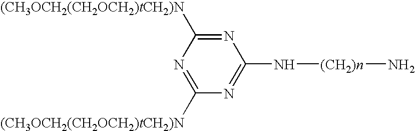
or 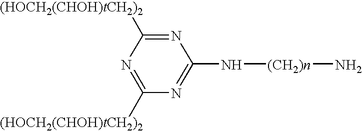
with t=1, 2, 3 or 4 and n=2 to 6.

53. Compound according to claim 19, wherein the cellular receptors or tissue components are chosen from receptors of myocardial cells, of endothelial cells, of epithelial cells, of tumour cells or of immune system cells.

54. Compound according to claim 22, wherein G3 is —CH—, G1 is OH, G6 is NH and K1 is —N(R_{f}4″)-.

55. Compound according to claim 24, wherein G1 is OH, G2 is NH₂, G6 is N.

56. Compound according to claim 31, wherein the biovector is chosen from an RGD peptide, a peptidomimetic of the RGD peptide, and a non-peptide agent capable of mimicing the action of an RGD peptide.

57. Compound according to claim 35, wherein the angiogenic receptor of endothelial cells is a VEGFR receptor.

58. Compound according to claim 35, wherein the biovector is a peptide ATWLPPR or HTMYYHHYQHHL.

59. Intermediate according to claim 41, wherein [(D)_{q}-(I_{a,b,c,d,e,f,g})ᵣ] preferably is chosen from: 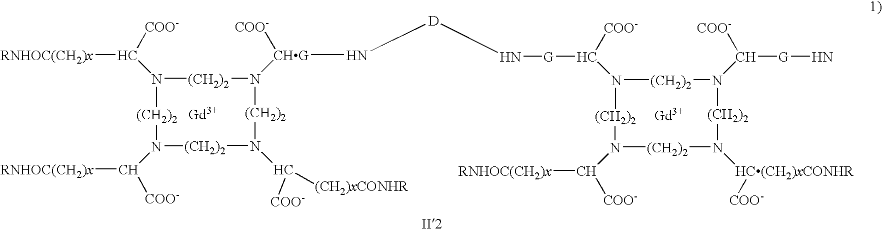
with -G-NH being —(CH₂)₃—NH— or 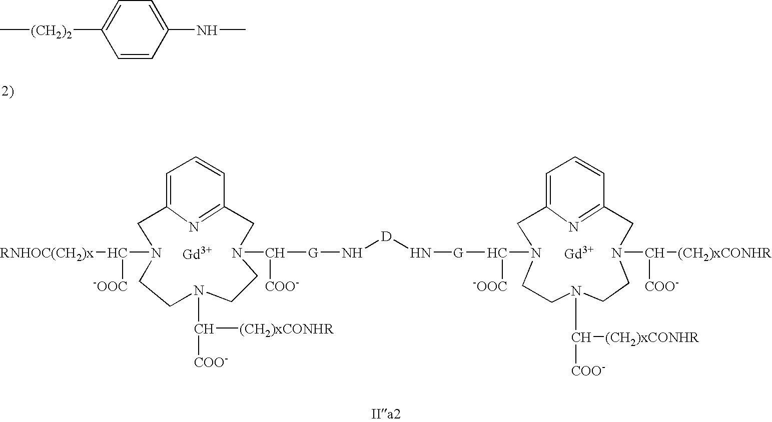 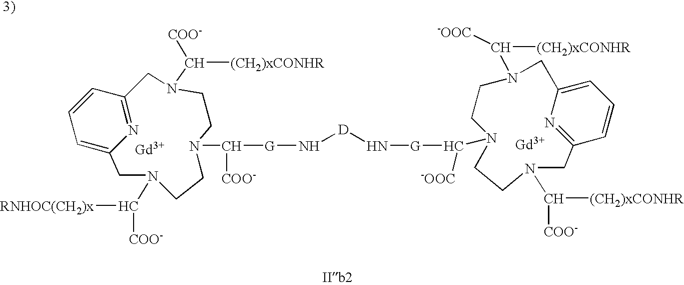
with -G-NH being —(CH₂)₃—NH— or

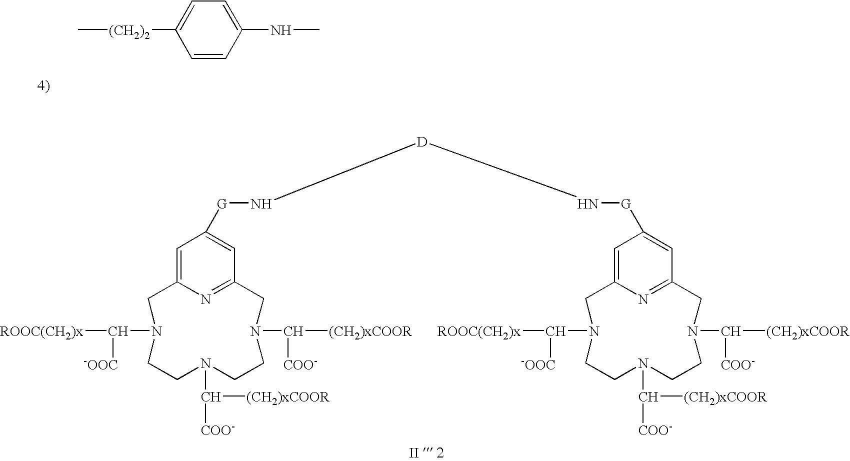

60. Method of diagnostic of a cardiovascular, cancer-related or inflammatory pathology comprising the administration of a magnetic resonance contrast product according to claim 43 to a patient in need thereof.
